(19) United States
(12) Patent Application Publication
(10) Pub. No.: US 2026/0061004 A1
(43) Pub. Date: Mar. 5, 2026

(54) NATURAL KILLER CELLS

(71) Applicants: IMPERIAL COLLEGE INNOVATIONS LIMITED, London (GB); NK:IO LTD, London (GB)

(72) Inventors: Ines ULLMO, London (GB); Hugh J.M. BRADY, London (GB); Matthew FUCHTER, London (GB); Heather ANG, London (GB); Joanna BURR, London (GB); Michelle GESTWA, London (GB); Nahide KOKSAL, London (GB)

(73) Assignees: IMPERIAL COLLEGE INNOVATIONS LIMITED, London (GB); NK:IO LTD, London (GB)

(21) Appl. No.: 19/107,401

(22) PCT Filed: Sep. 4, 2023
(86) PCT No.: PCT/GB2023/052282

§ 371 Date: Feb. 27, 2025

Related U.S. Application Data
(63) Continuation-in-part of application No. 17/929,417, filed on Sep. 2, 2022.

(30) Foreign Application Priority Data
Sep. 2, 2022 (GB) 2212838.3

(51) Int. Cl. A61K 35/17; A61K 40/15; A61K 40/42; A61K 45/06; C07K 14/735; C12N 5/00; C12N 5/0783

(52) U.S. Cl. CPC A61K 35/17; A61K 40/15; A61K 40/421; A61K 45/06; C07K 14/70535; C12N 5/0018; C12N 5/0646; C12N 2501/125; C12N 2501/145; C12N 2501/22; C12N 2501/2303; C12N 2501/2306; C12N 2501/2315; C12N 2501/26; C12N 2501/38; C12N 2501/42; C12N 2506/11; C12N 2506/45

(57) ABSTRACT

This invention relates to expanded Natural Killer (NK) cell populations, to methods of producing the same and therapeutic applications thereof. More specifically, the invention relates to increasing the number of CD16+ NK cells within expanded NK cell populations without the need for exogenous gene expression.

### FIELD OF THE INVENTION

This invention relates to expanded Natural Killer (NK) cell populations, to methods of producing the same and therapeutic applications thereof. More specifically, the invention relates to increasing the number of CD16⁺ NK cells within expanded NK cell populations without the need for exogenous gene expression.

### BACKGROUND OF THE INVENTION

There has been an increase in interest in Natural Killer (NK) cells as they are cytotoxic against cancerous, pathogen-infected and otherwise damaged cells. NK cells are innate lymphoid cells (ILCs), specifically large granular cytotoxic lymphocytes that bridge the innate and the adaptive arms of the immune response. They make up 10-15% of circulating lymphocytes in the peripheral blood. NK cells also exhibit the highest level of cytotoxic activity within the immune system. Therefore, altered NK cell functionality or numbers impact the functioning of the immune system against infection and cancer. For example, a large scale study in Japan has shown that reduced levels of NK cells in a cohort of people aged over 40 is associated with a significantly higher incidence of cancer.

Similarly to B cells and T cells, these NK cells are derived from Common Lymphoid Progenitor (CLP) cells that in turn come from Haematopoietic Stem Cells (HSCs). However, NK cells are different from B and T cells as they lack specific cell surface antigen receptors. Due to this, NK cells may kill cancerous and pathogen-infected cells without prior sensitisation, making them part of the innate immune response. They also have a critical role in tumour immunosurveillance by directly influencing the adaptive immune response.

Activation of NK cells triggers them to release perforin and cytoplasmic granules containing granzymes. Perforin polymerises to form pores on target cells in the presence of Ca²⁺. Granzymes may enter these pores into target cells, causing DNA fragmentation and apoptosis. NK cells may also secrete cytokines, which trigger the action of other immune cells in the adaptive arm of the immunity.

Numerous groups have worked on methods to increase the number of a patient's endogenous NK cells. One method is the administration of cytokines that are essential for NK cell development. Administration of IL-2 and IL-15 was predicted to enhance NK cell development. IL-2 promotes the proliferation and cytotoxicity of NK cells, whereas IL-15 promotes the development and expansion of NK cells. However, in in vivo studies, the cytokines were found only stimulate a minimal expansion of NK cells with reduced half-life, even at a very high dose. Further, administered cytokines often leads to systemic toxicity due to inappropriate activation of immune responses and the induction of NK cell apoptosis. In addition, even when previous methods have been able to increase NK cell expansion, the resulting expanded NK cells often exhibit limited functional activity, such as lower levels of IFNγ production and low levels of antibody-dependent cellular cytotoxicity (ADCC).

Thus, using conventional methods and techniques, producing large numbers of NK cells is difficult, and producing fully functional NK cells with high cytotoxicity is even harder. There is currently no drug available that selectively increases NK cell numbers, and particularly no agent that is available to produce large numbers of mature, active NK cells. Therefore, there is a need to develop new methods of NK cell production; both ex vivo to produce large numbers of functional NK cells for therapeutic and research use; and in vivo.

### SUMMARY OF THE INVENTION

Natural Killer (NK) cells have a critical role in the immune system where they destroy cancerous, pathogen-infected or damaged cells. Boosting NK cell number or functionality is predicted to increase the killing of these cells. Existing therapies such as NK cell adoptive transfer and cytokine enhancement of endogenous NK cells are not very successful in terms of their efficacy.

NK cells are differentiated from the HSCs in the bone marrow and distributed throughout lymphoid and non-lymphoid tissues including lymph nodes, spleen, peripheral blood, lungs and liver. Specific cytokines and transcription factors are needed to encourage HSCs to develop into NK cells. Each cytokine and transcription factor must be present at a precise time and concentration in order to push differentiation from HSCs into NK cells. However, the precise hierarchy of cytokines and transcription factors governing NK cell maturation is still incompletely understood.

The present inventors have previously shown that inhibiting the action of REV-ERB increases NK cell production. In particular, the inventors demonstrated that inhibiting the action of REV-ERB (e.g. using the REV-ERB antagonist SR8278) increases E4bp4 expression, which in turn increases NK cell production. The present inventors have also previously shown that abrogation of Notch signalling impedes NK cell production, and that the total lack of NK cell development from E4bp4^{-/-} progenitors can be completely rescued by short exposure to Notch peptide ligands, particularly Delta-like ligand 4 (DLL4). Further, the inventors have shown that combining these two independent mechanisms (use of Notch ligands and REV-ERB inhibition) results in a surprisingly potent means for enhancing NK cell production.

However, there is still a need to increase the proportion of allowing for the production of large numbers of fully functional NK cells that are suitable for in vivo therapeutic use. The inventors have now developed a new method for the expansion of NK cells that surprisingly increases the proportion of CD16⁺ NK cells. In particular, the inventors have surprisingly found that a production method which includes a distinct pre-differentiation culture stage give rise to NK cells with increased CD16 expression. Further, the inventors have also demonstrated that increasing the duration of this pre-differentiation stage beyond an upper time limit has a negative effect on CD16 expression. Therefore, there is a window of time for which pre-differentiation is advantageous, as it increases CD16 expression, but beyond which this advantage is reduced or lost. Without being bound by theory, it is believed that inclusion of a defined pre-differentiation stage according to the invention affects the transcriptome of the CD34+ HPCs, resulting in epigenetic changes which favour CD16 expression in CD56+ NK cells.

As CD16⁺ NK cells are critical for ADCC, the method of the present invention advantageously allow for the production of expanded NK cell populations with improved functionality. As a further advantage, the invention allows for the production of CD16⁺ NK cells without the need to introduce exogenous genetic elements into the NK cells, which provide a further significant benefit when the NK cells are intended for clinical use. Thus, the ex vivo method for increasing the number of CD16⁺ NK cells in an expanded NK population, and expanded NK cell populations made by said method provide a significant advantage, enabling the expansion of NK cells from haematopoietic progenitor cells (HPCs), which minimises cellular exhaustion and produces large numbers of functional CD16⁺ NK cells.

Accordingly, the present invention provides an ex vivo method for producing an expanded population of CD16+ Natural Killer (NK) cells, comprising the steps of: a) culturing an haematopoietic progenitor cell (HPC) comprising sample obtained from an individual in medium which does not induce differentiation of the HPCs for between about 2 to about 8 days to produce a pre-differentiation HPC population; and b) culturing the pre-differentiation HPC population in medium which induces differentiation of the HPCs to NK.

The invention also provides an ex vivo method for increasing the number of CD16+ NK cells in an expanded NK cell population, comprising the steps of: a) culturing an haematopoietic progenitor cell (HPC) comprising sample obtained from an individual in medium which does not induce differentiation of the HPCs for between about 2 to about 8 days to produce a pre-differentiation HPC population; and b) culturing the pre-differentiation HPC population in medium which induces differentiation of the HPCs to NK.

In step (a) the HPCs may be cultured for between about 2 days to about 6 days, optionally for between about 4 days to about 6 days.

The HPCs may be cultured in the presence of a Notch ligand for at least part of step (a).

The HPCs may be cultured in a vessel that is coated with the Notch ligand for at least part of step (a); and/or (ii) the Notch ligand may be delta-like ligand 4 (DLL4), or a fragment thereof which retains the function of DLL4. Preferably in step (a) the HPCs are cultured in the absence of the Notch ligand for about 1 day followed by culture in the presence of the Notch ligand for the remainder of step (a).

In step (a) the HPCs may be cultured in the presence of a compound which inhibits the action of REV-ERB; and/or in step (b) the pre-differentiation HPC population may be cultured in the presence of a compound which inhibits the action of REV-ERB.

Said compound may: (i) increase E4bp4 expression by decreasing REV-ERB activity; (ii) decrease the activity of REV-ERB-α and/or REV-ERB-β, preferably REV-ERB-β; (iii) decrease the activity of REV-ERB-α and REV-ERB-β; (iv) be a REV-ERB antagonist, preferably an antagonist of REV-ERB-α and REV-ERB-β; (v) be selected from a small molecule, a PROTAC reagent, a double stranded RNA (dsRNA), a small interfering RNA (siRNA), a small hairpin RNA (shRNA), a micro RNA, an antisense RNA, an aptamer, an antibody, a ribozyme, a peptide or a peptidomimetic, preferably a small molecule; and/or
(vi) be SR8278,

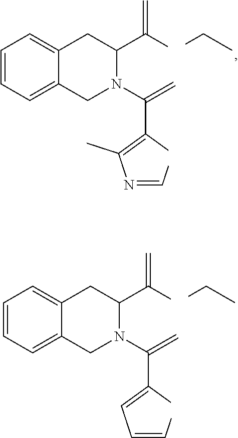 [SMILES: C=C(CCC)C1Cc2ccccc2CN1C(=C)C1=C(C)N=CC1 ; C=C(CCC)C1Cc2ccccc2CN1C(=C)C1=CC=CC1]

### or GSK1362.

The medium which does not induces differentiation of the HPCs in step (a) and/or the medium which induces differentiation of the HPCs to NK in step (b) may not comprise IL-3, preferably wherein the medium which induces differentiation of the HPCs to NK in step (b) does not comprise IL-3.

The medium in step (a) may comprise at least one of FIt3L, GM-CSF, IL-3, IL-6, TPO and/or stem cell factor (SCF), preferably FIt3L, GM-CSF, IL-3, IL-6, TPO and SCF. The medium in step (b) may comprise IL-7, FIt3L, IL-15, and/or SCF, preferably IL-7, FIt3L, IL-15 and SCF.

Step (a) and/or step (b) may be carried out in the absence of a stromal support cell, preferably wherein both step (a) and step (b) are carried out in the absence of a stromal support cell.

The sample of HPCs may be obtained from bone marrow, cord blood and/or peripheral blood.

The proportion of CD16+ NK cells may be increased compared with the proportion of CD16+ NK cells produced by a corresponding method in which step (a) is omitted. The expanded NK cell population may comprise at least 10% CD16+ NK cells, preferably at least 15% CD16+ NK cells, more preferably at least 20% CD16+ NK cells, even more preferably at least 30% CD16+ NK cells. The expanded NK cell population may exhibit at least 30% greater antibody-dependent cellular cytotoxicity (ADCC), preferably at least 50% greater ADCC, compared with NK cells produced by a corresponding method in which step (a) is omitted.

A method of the invention may not comprise a (further) step of introducing exogenous nucleic acid into the HPCs and/or NK cells.

The invention further provides an expanded population of CD16+ NK cells, wherein at least 10% of the NK cells are CD16+ NK cells, preferably at least 15% of the NK cells are CD16+ NK cells, more preferably at least 20% of the NK cells are CD16+ NK cells, even more preferably at least 30% of the NK cells are CD16+ NK cells.

The invention provides an expanded population of CD16+ NK cells obtained by the method of the invention, wherein at least 10% of the NK cells are CD16+ NK cells, preferably at least 15% of the NK cells are CD16+ NK cells, more preferably at least 20% of the NK cells are CD16+ NK cells, even more preferably at least 30% of the NK cells are CD16+ NK cells.

The CD16+ NK cells of the invention may not comprise exogenous nucleic acid.

The expanded NK cell population may exhibit at least 30% greater ADCC, preferably at least 50% greater ADCC, more preferably at least 70% greater ADCC, compared with NK cells produced by a corresponding method in which step (a) is omitted.

The invention further provides a composition comprising an expanded NK cell of the invention and a pharmaceutically acceptable carrier, diluent and/or excipient.

The invention also provides an expanded population of CD16+ NK or a composition of the invention for use in a method of therapy.

Said method of therapy may be a method of treating a disease or disorder selected from cancer, an infectious disease (acute or chronic), an autoimmune disease or a disease or disorder related to female infertility or pregnancy. Said method of therapy may be a method of treatment of a viral infection, a bacterial infection, a protist infection, a fungal infection and/or a helminth infection.

The invention also provides an expanded population of CD16+ NK cells or composition for use according to the invention, which is used in combination with antibody-mediated immunotherapy. Said compound may be for administration before, simultaneously with, or after administration of the antibody-mediated immunotherapy.

### BRIEF DESCRIPTION OF THE DRAWINGS

FIG. 1: NK cell developmental pathway. NK cells are differentiated from Hematopoietic Stem Cells (HSCs). NK cells develop from HSC into Common Lymphoid Progenitor (CLP) cells, NK progenitor (NKP) cells, immature NK (iNK) cells, mature NK (mNK) cells and finally into conventional NK (cNK) cells, which circulate in the bloodstream. Below the diagram of the pathway are the cytokines and transcription factors that are required for NK cell development. IL-15 is one of the main cytokines required for the development of NK cells and E4bp4 is a critical transcription factor directing the developmental programme. Others are transcription factors required for the transitions shown on the diagram. Eomes, Id2 and T-bet are additional transcription factors that have an important role in completing the maturation of NK cells.

FIG. 2: (A) Experimental timelines and flow cytometry data demonstrating that CD16 expression is increased when a set pre-differentiation step is included prior to differentiation. The flow cytometry data shows the cell surface levels of CD56 and CD16 on cells following 20 days culture in NK cell differentiation conditions. (B) absolute number of human CD56+CD16− and CD56+CD16+ NK cells generated after 20 days differentiation on stromal cells. DO, D-2, D-4 & D-6 refers to the time the cells spent in pre-differentiation before being placed on stromal cells. (C) number of NK cells produced at different lengths of time differentiating on stromal cells plotted against the number of days of pre-differentiation.

FIG. 3: Experimental timelines and flow cytometry data demonstrating that addition of DLL4 further increases CD16 expression when combined with a set pre-differentiation step.

FIG. 4: Graphs showing % and absolute number of CD56+ NK cells over time (A) and % and absolute number of CD56⁺CD16⁺ NK cells over time (B) in the presence or absence of DLL4 with set pre-differentiation steps of different lengths. ●=+DLL4; ▴=no DLL4 FIG. 5: Graph showing the effect of IL-3 on the % of CD56+CD16⁺ NK cells over time when HPCs are cultured in the presence/absence of DLL4 and/or presence/absence of EL08 stromal cells.

FIG. 6: Flow cytometry data demonstrating that increasing the duration of the pre-differentiation step from 4 days to 14 days significantly reduces the number of CD56⁺CD45+ NK cells produced and effectively reduces the number CD56⁺CD16⁺ NK cells produced to zero.

FIG. 7: Flow cytometry data showing CD56⁺CD45+ NK cell numbers produced by a method of the invention comprising (top left) a 4-day pre-differentiation step; (top right) a 4-day pre-differentiation step+REV-ERB inhibitor; (bottom left) a 4-day pre-differentiation step+DLL4; and (bottom right) a 4-day pre-differentiation step+REV-ERB inhibitor+DLL4. The REV-ERB inhibitor and DLL4 both increase CD56⁺CD45+ NK cell numbers compared with a 4-day pre-differentiation step alone. Combining both REV-ERB inhibitor and DLL4 with a 4-day pre-differentiation step elicits the greatest increase in CD56⁺CD45+ NK cell number.

FIG. 8: Flow cytometry data demonstrating the expression of KIRs (KIR2DL1, KIR2DS1, KIR2DS3, KIR2DS5) detected using anti-CD158 antibody on CD56⁺CD16⁺ NK cells and CD56⁺CD16⁻ NK cells produced by a method of the invention using a 6-day pre-differentiation period and a 23-day differentiation period.

### DETAILED DESCRIPTION OF THE INVENTION

### Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure. The meaning and scope of the terms should be clear; however, in the event of any latent ambiguity, definitions provided herein take precedent over any dictionary or extrinsic definition.

It should be understood that this invention is not limited to the particular methodology, protocols, and reagents, etc., described herein and as such can vary. In particular, any methods and materials similar or equivalent to those described herein can be used in the practice or testing of embodiments of this disclosure.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

As used herein, the term “capable of’ when used with a verb, encompasses or means the action of the corresponding verb. For example, “capable of interacting” also means interacting, “capable of cleaving” also means cleaves, “capable of binding” also means binds and “capable of specifically targeting . . . ” also means specifically targets.

As used herein, the term “CD16⁺” refers to cells which are CD16⁺ and/or CD16^{hi}, i.e. cells which are positive for and/or express high-levels of CD16.

Numeric ranges are inclusive of the numbers defining the range. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

Amino acids are referred to herein using the name of the amino acid, the three-letter abbreviation or the single letter abbreviation.

As used herein, the terms “protein” and “polypeptide” are used interchangeably herein to designate a series of amino acid residues, connected to each other by peptide bonds between the alpha-amino and carboxyl groups of adjacent residues. The terms “protein”, and “polypeptide” refer to a polymer of amino acids, including modified amino acids (e.g., phosphorylated, glycated, glycosylated, etc.) and amino acid analogues, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogues of the foregoing. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Minor variations in the amino acid sequences of the invention are contemplated as being encompassed by the present invention, providing that the variations in the amino acid sequence(s) maintain at least 60%, at least 70%, more preferably at least 80%, at least 85%, at least 90%, at least 95%, and most preferably at least 97% or at least 99% sequence identity to the amino acid sequence of the invention or a fragment thereof as defined anywhere herein. The term homology is used herein to mean identity. As such, the sequence of a variant or analogue sequence of an amino acid sequence of the invention may differ on the basis of substitution (typically conservative substitution) deletion or insertion. Proteins comprising such variations are referred to herein as variants.

Proteins of the invention may include variants in which amino acid residues from one species are substituted for the corresponding residue in another species, either at the conserved or non-conserved positions. Variants of protein molecules disclosed herein may be produced and used in the present invention. Following the lead of computational chemistry in applying multivariate data analysis techniques to the structure/property-activity relationships [see for example, Wold, et al. Multivariate data analysis in chemistry. Chemometrics-Mathematics and Statistics in Chemistry (Ed.: B. Kowalski); D. Reidel Publishing Company, Dordrecht, Holland, 1984 (ISBN 90-277-1846-6] quantitative activity-property relationships of proteins can be derived using well-known mathematical techniques, such as statistical regression, pattern recognition and classification [see for example Norman et al. Applied Regression Analysis. Wiley-Interscience; 3rd edition (April 1998) ISBN: 0471170828; Kandel, Abraham et al. Computer-Assisted Reasoning in Cluster Analysis. Prentice Hall PTR, (May 11, 1995), ISBN: 0133418847; Krzanowski, Wojtek. Principles of Multivariate Analysis: A User's Perspective (Oxford Statistical Science Series, No 22 (Paper)). Oxford University Press; (December 2000), ISBN: 0198507089; Witten, Ian H. et al Data Mining: Practical Machine Learning Tools and Techniques with Java Implementations. Morgan Kaufmann; (Oct. 11, 1999), ISBN:1558605525; Denison David G. T. (Editor) et al Bayesian Methods for Nonlinear Classification and Regression (Wiley Series in Probability and Statistics). John Wiley & Sons; (July 2002), ISBN: 0471490369; Ghose, Arup K. et al. Combinatorial Library Design and Evaluation Principles, Software, Tools, and Applications in Drug Discovery. ISBN: 0-8247-0487-8]. The properties of proteins can be derived from empirical and theoretical models (for example, analysis of likely contact residues or calculated physicochemical property) of proteins sequence, functional and three-dimensional structures and these properties can be considered individually and in combination.

Amino acid residues at non-conserved positions may be substituted with conservative or non-conservative residues. In particular, conservative amino acid replacements are contemplated.

A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. The inclusion of conservatively modified variants in a protein of the invention does not exclude other forms of variant, for example polymorphic variants, interspecies homologs, and alleles.

“Non-conservative amino acid substitutions” include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, lie, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

“Insertions” or “deletions” are typically in the range of about 1, 2, or 3 amino acids. The variation allowed may be experimentally determined by systematically introducing insertions or deletions of amino acids in a protein using recombinant DNA techniques and assaying the resulting recombinant variants for activity. This does not require more than routine experiments for a skilled person.

A “fragment” of a polypeptide typically comprises at least 50%, at least 60%, at least 70%, at least 80%, at least 90%, at least 95%, at least 97% or more of the original polypeptide.

As used herein, the terms “polynucleotides”, “nucleic acid” and “nucleic acid sequence” refers to any molecule, preferably a polymeric molecule, incorporating units of ribonucleic acid, deoxyribonucleic acid or an analogue thereof. The nucleic acid can be either single-stranded or double-stranded. A single-stranded nucleic acid can be one nucleic acid strand of a denatured double-stranded DNA Alternatively, it can be a single-stranded nucleic acid not derived from any double-stranded DNA. In one aspect, the nucleic acid can be DNA. In another aspect, the nucleic acid can be RNA Suitable nucleic acid molecules are DNA, including genomic DNA or cDNA. Other suitable nucleic acid molecules are RNA, including siRNA, shRNA, and antisense oligonucleotides. The terms “transgene” and “gene” are also used interchangeably and both terms encompass fragments or variants thereof encoding the target protein.

The polynucleotides of the present invention include nucleic acid sequences that have been removed from their naturally occurring environment, recombinant or cloned DNA isolates, and chemically synthesized analogues or analogues biologically synthesized by heterologous systems.

The polynucleotides of the present invention may be prepared by any means known in the art. For example, large amounts of the polynucleotides may be produced by replication in a suitable host cell. The natural or synthetic DNA fragments coding for a desired fragment will be incorporated into recombinant nucleic acid constructs, typically DNA constructs, capable of introduction into and replication in a prokaryotic or eukaryotic cell. Usually, the DNA constructs will be suitable for autonomous replication in a unicellular host, such as yeast or bacteria, but may also be intended for introduction to and integration within the genome of a cultured insect, mammalian, plant or other eukaryotic cell lines.

The polynucleotides of the present invention may also be produced by chemical synthesis, e.g. by the phosphoramidite method or the tri-ester method, and may be performed on commercial automated oligonucleotide synthesizers. A double-stranded fragment may be obtained from the single stranded product of chemical synthesis either by synthesizing the complementary strand and annealing the strand together under appropriate conditions or by adding the complementary strand using DNA polymerase with an appropriate primer sequence.

When applied to a nucleic acid sequence, the term “isolated” in the context of the present invention denotes that the polynucleotide sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment.

In view of the degeneracy of the genetic code, considerable sequence variation is possible among the polynucleotides of the present invention. Degenerate codons encompassing all possible codons for a given amino acid are set forth below:

| Amino Acid | Codons | Degenerate Codon |
|---|---|---|
| Cys | TGC TGT | TGY |
| Ser | AGC AGT TCA TCC TCG TCT | WSN |
| Thr | ACA ACC ACG ACT | ACN |
| Pro | CCA CCC CCG CCT | CCN |
| Ala | GCA GCC GCG GCT | GCN |
| Gly | GGA GGC GGG GGT | GGN |
| Asn | AAC AAT | AAY |
| Asp | GAC GAT | GAY |
| Glu | GAA GAG | GAR |
| Gln | CAA CAG | CAR |
| His | CAC CAT | CAY |
| Arg | AGA AGG CGA CGC CGG CGT | MGN |
| Lys | AAA AAG | AAR |
| Met | ATG | ATG |
| Ile | ATA ATC ATT | ATH |
| Leu | CTA CTC CTG CTT TTA TTG | YTN |
| Val | GTA GTC GTG GTT | GTN |
| Phe | TTC TTT | TTY |
| Tyr | TAC TAT | TAY |
| Trp | TGG | TGG |
| Ter | TAA TAG TGA | TRR |
| Asn/Asp | | RAY |
| Glu/Gln | | SAR |
| Any | | NNN |

One of ordinary skill in the art will appreciate that flexibility exists when determining a degenerate codon, representative of all possible codons encoding each amino acid. For example, some polynucleotides encompassed by the degenerate sequence may encode variant amino acid sequences, but one of ordinary skill in the art can easily identify such variant sequences by reference to the amino acid sequences of the present invention.

A “variant” nucleic acid sequence has substantial homology or substantial similarity to a reference nucleic acid sequence (or a fragment thereof). A nucleic acid sequence or fragment thereof is “substantially homologous” (or “substantially identical”) to a reference sequence if, when optimally aligned (with appropriate nucleotide insertions or deletions) with the other nucleic acid (or its complementary strand), there is nucleotide sequence identity in at least about 70%, 75%, 80%, 85, 90, 91, 92, 93, 94, 95, 96, 97, 98, 99 or more % of the nucleotide bases. Methods for homology determination of nucleic acid sequences are known in the art.

Alternatively, a “variant” nucleic acid sequence is substantially homologous with (or substantially identical to) a reference sequence (or a fragment thereof) if the “variant” and the reference sequence they are capable of hybridizing under stringent (e.g. highly stringent) hybridization conditions. Nucleic acid sequence hybridization will be affected by such conditions as salt concentration (e.g. NaCl), temperature, or organic solvents, in addition to the base composition, length of the complementary strands, and the number of nucleotide base mismatches between the hybridizing nucleic acids, as will be readily appreciated by those skilled in the art. Stringent temperature conditions are preferably employed, and generally include temperatures in excess of 30° C., typically in excess of 37° C. and preferably in excess of 45° C. Stringent salt conditions will ordinarily be less than 1000 mM, typically less than 500 mM, and preferably less than 200 mM. The pH is typically between 7.0 and 8.3. The combination of parameters is much more important than any single parameter.

Methods of determining nucleic acid percentage sequence identity are known in the art. By way of example, when assessing nucleic acid sequence identity, a sequence having a defined number of contiguous nucleotides may be aligned with a nucleic acid sequence (having the same number of contiguous nucleotides) from the corresponding portion of a nucleic acid sequence of the present invention. Tools known in the art for determining nucleic acid percentage sequence identity include Nucleotide BLAST (as described below).

One of ordinary skill in the art appreciates that different species exhibit “preferential codon usage”. As used herein, the term “preferential codon usage” refers to codons that are most frequently used in cells of a certain species, thus favouring one or a few representatives of the possible codons encoding each amino acid. For example, the amino acid threonine (Thr) may be encoded by ACA, ACC, ACG, or ACT, but in mammalian host cells ACC is the most commonly used codon; in other species, different codons may be preferential. Preferential codons for a particular host cell species can be introduced into the polynucleotides of the present invention by a variety of methods known in the art. Introduction of preferential codon sequences into recombinant DNA can, for example, enhance production of the protein by making protein translation more efficient within a particular cell type or species. Thus, according to the invention, in addition to the gag-pol genes any nucleic acid sequence may be codon-optimised for expression in a host or target cell. In particular, the vector genome (or corresponding plasmid), the REV gene (or corresponding plasmid), the fusion protein (F) gene (or correspond plasmid) and/or the hemagglutinin-neuraminidase (HN) gene (or corresponding plasmid, or any combination thereof may be codon-optimised.

A “fragment” of a polynucleotide of interest comprises a series of consecutive nucleotides from the sequence of said full-length polynucleotide. By way of example, a “fragment” of a polynucleotide of interest may comprise (or consist of) at least 30 consecutive nucleotides from the sequence of said polynucleotide (e.g. at least 35, 50, 75, 100, 150, 200, 250, 300, 350, 400, 450, 500, 550, 600, 650, 700, 750, 800 850, 900, 950 or 1000 consecutive nucleic acid residues of said polynucleotide). A fragment may include at least one antigenic determinant and/or may encode at least one antigenic epitope of the corresponding polypeptide of interest. Typically, a fragment as defined herein retains the same function as the full-length polynucleotide.

The terms “increased”, “increase”, “enhance”, or “activate” are all used herein to mean an increase by a statically significant amount. The terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a yield or titre, an “increase” is an observable or statistically significant increase in such level.

The terms “decrease”, “reduced”, “reduction”, or “inhibit” are all used herein to mean a decrease by a statistically significant amount. The terms “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” encompasses a complete inhibition or reduction as compared to a reference level. “Complete inhibition” is a 100% inhibition (i.e. abrogation) as compared to a reference level.

It must be noted that as used herein and in the appended claims, the singular forms “a”, “an”, and “the” include plural referents unless the context clearly dictates otherwise. Thus, for example, reference to “a Notch ligand” includes a plurality of such agents and reference to “the Notch ligand” includes reference to one or more Notch ligand and equivalents thereof known to those skilled in the art, and so forth. Furthermore, the use of the term “including”, as well as other forms, such as “includes” and “included”, is not limiting.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (±) 5%, preferably ±4%, ±3%, ±2%, ±1%, ±0.5%, ±0.1%, of the numerical value of the number with which it is being used.

The term “consisting of” refers to compositions, methods, and respective components thereof as described herein, which are exclusive of any element not recited in that description of the invention.

As used herein the term “consisting essentially of” refers to those elements required for a given invention. The term permits the presence of elements that do not materially affect the basic and novel or functional characteristic(s) of that invention (i.e. inactive or non-immunogenic ingredients).

Embodiments described herein as “comprising” one or more features may also be considered as disclosure of the corresponding embodiments “consisting of” and/or “consisting essentially of” such features.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

The terms “individual”, “subject”, and “patient”, are used interchangeably herein to refer to a mammalian subject for whom diagnosis, prognosis, disease monitoring, treatment, therapy, and/or therapy optimisation is desired. The mammal can be (without limitation) a human, non-human primate, mouse, rat, dog, cat, horse, or cow. In a preferred embodiment, the individual, subject, or patient is a human. An “individual” may be an adult, juvenile or infant. An “individual” may be male or female.

A “subject in need” of treatment for a particular condition can be an individual having that condition, diagnosed as having that condition, or at risk of developing that condition.

A subject can be one who has been previously diagnosed with or identified as suffering from or having a condition in need of treatment or one or more complications related to such a condition, and optionally, have already undergone treatment for a condition as defined herein or the one or more complications related to said condition. Alternatively, a subject can also be one who has not been previously diagnosed as having a condition as defined herein or one or more complications related to said condition. For example, an individual can be one who exhibits one or more risk factors for a condition, or one or more complications related to said condition or a subject who does not exhibit risk factors.

As used herein, the term “healthy individual” refers to an individual or group of individuals who are in a healthy state, e.g. individuals who have not shown any symptoms of the disease, have not been diagnosed with the disease and/or are not likely to develop the disease e.g. cancer or any other disease described herein). Preferably said healthy individual(s) is not on medication affecting cancer and has not been diagnosed with any other disease. The one or more healthy individuals may have a similar sex, age, and/or body mass index (BMI) as compared with the test individual. Application of standard statistical methods used in medicine permits determination of normal levels of expression in healthy individuals, and significant deviations from such normal levels.

Herein the terms “control” and “reference population” are used interchangeably.

The term “pharmaceutically acceptable” as used herein means approved by a regulatory agency of the Federal or a state government, or listed in the U.S. Pharmacopeia, European Pharmacopeia or other generally recognized pharmacopeia.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

The publications discussed herein are provided solely for their disclosure prior to the filing date of the present application. Nothing herein is to be construed as an admission that such publications constitute prior art to the claims appended hereto. All references cited in this specification are herewith incorporated by reference with respect to their entire disclosure content and the disclosure content specifically mentioned in this specification.

Disclosure related to the various methods of the invention are intended to be applied equally to other methods, therapeutic uses or methods, and vice versa.

### Natural Killer Cells

Natural Killer (NK) cells exhibit the highest level of cytotoxic activity within the immune system. NK cells are similar to B cells and T cells, but lack specific cell surface antigen receptors. Instead, NK cells have activatory and inhibitory receptors that recognise motifs.

NK cells circulate in the blood and the peripheral lymphoid organs such as lymph nodes and spleen. They can become activated by cytokines or upon encountering target cells. The recognition and elimination of target cells is based on balancing between inhibitory and activatory signals. Activatory signals are generated by activatory receptors (NKG2D, NKp46, NKp30) binding to ligands, which can be present not only on cancerous, pathogen-infected and damaged cells, but also on healthy cells. On the other hand, inhibitory signals are generated when inhibitory receptors (KIR, CD94/NKG2A) on NK cells bind to Major Histocompatability Complex (MHC) Class I molecules that are normally present on all healthy cells. MHC Class I molecules on target cells are absent or greatly downregulated, making them ideal NK cell targets. This allowed NK cells to distinguish between target and healthy cells. In order for NK cells to recognise and kill target cells, overall activatory signals must be greater than inhibitory signals.

NK cells recognise and kill cancerous, pathogen-infected and damaged cells without prior sensitisation, making them part of the innate immune response. For example, NK cells provide an early response to virus infection, occurring prior to T cell killing of infected cells. NK cells can kill target cells within minutes. NK cells also secrete cytokines and “weaponise” other parts of the immune system. For example, NK cells promote T cell effector function and enhance antibody-directed cellular cytotoxicity (ADCC).

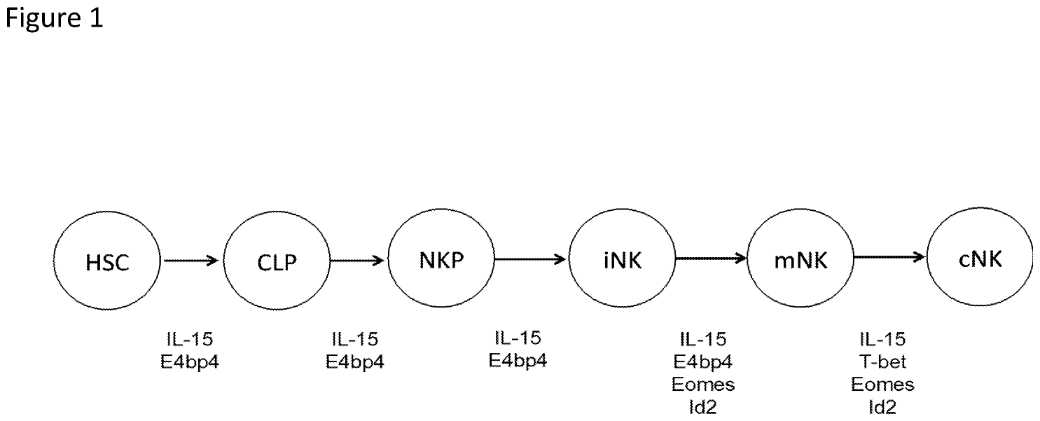

NK cells are differentiated from haematopoietic stem cells (HSCs) via the pathway set out in FIG. 1. In more detail, NK cells develop from HSCs into Common Lymphoid Progenitor (CLP) cells, pre-NK progenitor (pre-NKP) cells, NK progenitor (NKP) cells, immature NK (iNK) cells, mature NK (mNK) cells and finally into conventional NK (cNK) cells, which circulate in the bloodstream. Although this terminology derives from NK cell development in mice, a corresponding pathway occurs in human NK cell development. For example, HSCs develop through multiple stages of precursors (stage 1, 2 and 3), before developing into mature NK cells (stages 4 and 5). For consistency, references HSCs, CLPs, pre-NKPs, NKPs, iNK, mNK, cNK and NK cells are used herein. However, in the context of the present invention, these terms are interchangeable with stages 1 to 5 of the human nomenclature. Below the diagram of the pathway in FIG. 1 are the cytokines and transcription factors that are essential for NK cell development. IL-15 is one of the main cytokine required for the development of NK cells. Other extrinsic factors, such as specific stromal cells, are also required for the development and maturation of NK cells.

According to the present invention, Hematopoietic Progenitor Cells (HPCs) are a heterogeneous population containing multi-potential progenitors such as HSCs, CLPs and also NKPs. HPCs are referred to as lineage negative cells, as they have not yet committed to a developmental pathway. Accordingly, in the context of the present invention, HSCs, CLP cells and NKP cells are all HPCs and a reference to HPCs is a reference to any of HSCs, CLP cells and/or CLP cells, or any combination thereof, unless explicitly stated to the contrary.

Due to the importance of NK cells in immune response, multiple clinical trials have tested the efficacy of NK cells in adoptive transfer protocols. Typically this is allogenic transfer, with the NK cells being isolated from a healthy donor and expanded. However, the downregulation of MHC Class I molecules on target cells is partial and the KIR genotype from donors and recipients may be similar. Due to this, NK cells transfused into recipients, even from different individuals may not attach target cells if their KIRs recognise MHC Class I molecules. Therefore, it is crucial that NK cell donors must be screened for their KIR genotype, where the donor must have the appropriate KIR allelic polymorphism to the recipient to allow recognition of target cells for destruction. Moreover, the expanded products were found to have lower clinical success rate than expected, with less ability to kill cancerous or infected cells.

An NK cell may be defined in terms of its marker expression, its function/activity, or a combination thereof. Such definitions are standard in the art and methods are known by which marker expression and/or NK cell activity may be assessed. Thus, one of skill in the art would readily be able to categorise a cell as an NK cell using standard methodology and definitions.

For example, mNK and cNK cells may be recognised by their expression of the surface markers CD16 (FcγRIII) and/or CD56, typically both CD16 and CD56 in humans, and NK1.1 or NK1.2 in some mice strains. NKp46 is another marker for mNK and cNK cells, and is expressed in humans and several mice strains. Thus, NKp46 may be used as a marker for NK cells either with or without CD16 and/or CD56 (in humans) or with or without NK1.1 or NK1.2 (in mice). Other examples of makers which can be used to identify/define NK cells according to the present invention include Ly49, natural cytotoxicity receptors (NCRs), CD94, NKG2, killer-cell immunoglobulin-like receptors (KIRs), and/or leukocyte inhibitory receptors (ILT or LIR), or any combination thereof, including in combination with CD16 and or CD56 (in humans) or NK1.1/NK1.2 (in mice). Mature NK cells according to the invention (i.e. mNK and cNK cells) are CD56⁺ and CD45⁺, and may be also be CD16⁺. As used herein, the term mature human NK cell encompasses NK cells that are CD56^{bright} (stage 4) and CD56^{dim} (stage 5), both of which are CD56⁺. Mature NK cells may also be defined by the absence of markers, such as CD34, and lymphocyte markers CD3 and/or CD19. Thus, mature NK cells of the invention may be CD56⁺, CD45⁺, CD16⁺, CD3⁻ and/or CD19⁻, or any combination thereof, such as CD56⁺, CD45⁺, CD16⁺, CD3⁻ and CD19⁻.

In addition to having increased CD16 expression as described herein, the (mature) NK cells of the invention are typically at least 80% CD56+ or CD56^{bright} such as at least 81% CD56+ or CD56^{bright} 85% CD56+ or CD56^{bright}, at least 86% CD56+ or CD56^{bright}, at least 87% CD56+ or CD56^{bright} at least 88% CD56+ or CD56^{bright}, at least 89% CD56+ or CD56^{bright}, at least 90% CD56+ or CD56^{bright} at least 91% CD56+ or CD56^{bright}, at least 92% CD56+ or CD56^{bright}, at least 93% CD56+ or CD56^{bright} at least 94% CD56+ or CD56^{bright}, at least 95% CD56+ or CD56^{bright}, at least 96% CD56+ or CD56^{bright} at least 97% CD56+ or CD56^{bright}, at least 98% CD56+ or CD56^{bright} at least 99% CD56+ or CD56^{bright} or more, up to 100% CD56+ or CD56^{bright}.

In addition to having increased CD16 expression as described herein, in an expanded population of NK cells (typically mature NK cells) provided by the invention, at least 50% of the NK cells in the population may be CD56+ or CD56^{bright} such as at least 60% of the NK cells in the population may be CD56+ or CD56^{bright}, at least 70% of the NK cells in the population may be CD56+ or CD56^{bright} at least 80% of the NK cells in the population may be CD56+ or CD56^{bright}, or at least 90% of the NK cells in the population may be CD56+ or CD56^{bright} or more. Typically, in an expanded population of NK cells (typically mature NK cells) provided by the invention at least 80% of the NK cells in the population are CD56+ or CD56^{bright}. For example, at least 80% of the NK cells in the population may be CD56+ or CD56^{bright} at least 81% of the NK cells in the population may be CD56+ or CD56^{bright} at least 85% of the NK cells in the population may be CD56+ or CD56^{bright}, at least 80% of the NK cells in the population may be CD56+ or CD56^{bright}, at least 86% of the NK cells in the population may be CD56+ or CD56^{bright}, at least 87% of the NK cells in the population may be CD56+ or CD56^{bright}, at least 88% of the NK cells in the population may be CD56+ or CD56^{bright}, at least 89% of the NK cells in the population may be CD56+ or CD56^{bright}, at least 90% of the NK cells in the population may be CD56+ or CD56^{bright} at least 91% of the NK cells in the population may be CD56+ or CD56^{bright}, at least 92% of the NK cells in the population may be CD56+ or CD56^{bright}, at least 93% of the NK cells in the population may be CD56+ or CD56^{bright} at least 94% of the NK cells in the population may be CD56+ or CD56^{bright} at least 95% of the NK cells in the population may be CD56+ or CD56^{bright}, at least 96% of the NK cells in the population may be CD56+ or CD56^{bright}, at least 97% of the NK cells in the population may be CD56+ or CD56^{bright}, at least 98% of the NK cells in the population may be CD56+ or CD56^{bright}, at least 99% of the NK cells in the population may be CD56+ or CD56^{bright} or more, up to 100% of the NK cells in the population may be CD56+ or CD56^{bright}.

These high proportions of CD56 expression may be achieved by the methods of the invention without or before any purification and/or concentration step. Alternatively, these high proportions of CD56 expression may be achieved by the methods of the invention following purification and/or concentration. Typically, the number of purification and/or concentration steps and/or the number of purification and/or concentration techniques used to achieve these high proportions of CD56 expression in an expanded NK cell population according to the invention is lower than that required by the prior art methods. By way of non-limiting example, a negative selection step may be used to purify an expanded NK cell population produced by a method of the invention.

Such expanded NK cell populations can typically be obtained by a method of the invention. CD56 expression may be determined using standard techniques, examples of which are known in the art and could be routinely selected by one of ordinary skill. Non-limiting examples of suitable techniques include flow cytometry, cell imaging and ELISA.

In addition or alternatively, an NK cell of the invention, or an expanded population of NK cells (typically mature NK cells) of the invention, may express one or more killer-cell immunoglobulin-like receptors (KIRs), particularly one or more of KIR2DL1, KIR2DS1, KIR2DS3 and/or KIR2DS5 i.e. may be KIR2DL1⁺ or KIR2DL1^{hi}; KIR2DS1⁺ or KIR2DS1^{hi}; KIR2DS3⁺ or KIR2DS3^{hi}; and/or KIR2DS5⁺ or KIR2DS5^{hi} The level of expression of expression of one or more of KIR2DL1, KIR2DS1, KIR2DS3 and/or KIR2DS5 may be increased compared with the level of the same KIR expressed by a suitable reference or control NK cell or NK cell population, such as a reference or control NK or NK cell population made by a control method as described herein. By way of example, KIR expression in the CD56⁺CD16⁺ NK cells of the invention may express increased KIR2DL1, KIR2DS1, KIR2DS3 and/or KIR2DS5 compared with CD56⁺CD16⁻ NK cells produced by the same method.

Alternatively or in addition, an NK cell of the invention, or an expanded population of NK cells (typically mature NK cells) of the invention may not express, or may express a low level of KIR2DL4, i.e. may be KIR2DL4⁻ or KIR2DL4^{lo}. The level of expression of expression of KIR2DL4 may be decreased compared with the level of KIR2DL4 expressed by a suitable reference or control NK cell or NK cell population, such as a reference or control NK or NK cell population made by a control method as described herein. Without being bound by theory, it is believed that expression of high levels of KIR2DL4 is disadvantageous, as KIR2DL4 is an inhibitory receptor which reduces NK cell function when binding to its ligand, HLA-G. For example, in ovarian cancer, some tumours over express HLA-G to inhibit the cancer cell killing function of NK cells.

In addition or alternatively, an NK cell may be identified by/defined in terms of its activity. For example, an NK cell may be identified/defined by the presence of cytolytic granules within its cytoplasm, by its ability to secrete antimicrobial molecules such as α-defensins, and/or its ability to secrete cytokines such as TNF-α, IL-10, IFN-γ and TFG-β.

Unless otherwise stated herein, a reference to NK cells includes a reference to iNK, mNK and cNK cells. HSCs, CLP cells and NKPs will typically be referred to as such.

As described herein, one benefit of the invention is that it provides NK cells which have increased CD16 expression compared with conventional methods for NK cell production, and which: (i) do not contain exogenous nucleic acid; and/or (ii) which have not undergone extensive purification and/or concentration, which can result in damage to the purified and/or concentrated NK cells. Alternatively or in addition, the NK cells of the present invention may possess further phenotypic differences compared to those produced by prior art methods, such as the expression of one or more distinguishing marker, or a distinguishing marker profile as described above. These differences arise as a result of the method of the invention, such that NK cells produced by a method of the present invention differ in key characteristics compared with NK cells produced by prior art methods.

### CD16 and Antibody-Dependent Cellular Cytotoxicity (ADCC)

CD16 (FcγRIII) is an Fc receptor expressed on the surface of NK cells, neutrophils, monocytes, macrophages, and some T cells. CD16A (FcγRIIIA) is expressed by NK cells. The human CD16A protein (UniProt Accession No. P08637, sequence version 2, accessed 26 May 2022) is expressed on NK cells. The terms CD16, CD16A, FcγRIII and FcγRIIIA are used interchangeably herein.

CD16 signals through association with the Fc receptor common γ chain, which possesses immunoreceptor tyrosine-based activation motifs (ITAMs). Signalling through CD16 mediates ADCC by NK cells. In ADCC the Fc regions of antibodies bound to the surface of target cells are recognised and bound by CD16 on the surface of NK cells. Cross-linking of CD16 with the antibody Fc regions activates the NK cells, resulting in the release of cytotoxic factors by the NK cells (also known as degranulation) that cause the death of the target cell by apoptosis. Therefore, NK cells with increased CD16 expression exhibit increased ADCC compared with NK cells with no or low CD16 expression.

The present inventors have developed an ex vivo method for expanding NK cells which also increases the number of CD16⁺ NK cells within the resulting expanding NK cell population.

Therefore the invention provides mature NK cells, and particular expanded populations of NK cells (typically mature NK cells) which are CD56⁺, CD45⁺, CD3⁻ and/or CD19⁻ (e.g. CD56⁺, CD45⁺, CD3⁻ and CD19⁻) and which also have increased CD16⁺ expression compared with a control expanded NK cell population. A control expanded NK cell population may be one that is produced by any conventional method for NK cell expansion, or a corresponding method to that described herein, but without the pre-differentiation step (step (a)).

Therefore, the invention provides an expanded population of NK cells, wherein at least 10% of the NK cells in the population are CD16⁺ NK cells, at least 15% of the NK cells in the population are CD16⁺ NK cells, at least 20% of the NK cells in the population are CD16⁺ NK cells, at least 25% of the NK cells in the population are CD16⁺ NK cells, at least 30% of the NK cells in the population are CD16⁺ NK cells, at least 35% of the NK cells in the population are CD16⁺ NK cells, at least 40% of the NK cells in the population are CD16⁺ NK cells, at least 45% of the NK cells in the population are CD16⁺ NK cells, at least 50% of the NK cells in the population are CD16⁺ NK cells, at least 60% of the NK cells in the population are CD16⁺ NK cells, at least 70% of the NK cells in the population are CD16⁺ NK cells, at least 80% of the NK cells in the population are CD16⁺ NK cells, up to 100% of the NK cells in the population are CD16⁺ NK cells. Preferably at least 15% of the NK cells are CD16⁺ NK cells, more preferably at least 20% of the NK cells are CD16⁺ NK cells, still more preferably at least 25% of the NK cells are CD16⁺ NK cells even more preferably at least 30% of the NK cells are CD16⁺ NK cells, even more preferably at least 40% of the NK cells are CD16⁺ NK cells. Such expanded NK cell populations can typically be obtained by a method of the invention. CD16 expression may be determined using standard techniques, examples of which are known in the art and could be routinely selected by one of ordinary skill. Non-limiting examples of suitable techniques include flow cytometry, cell imaging and ELISA.

One benefit of the CD16⁺ NK cells of the invention is that they exhibit increased CD16 expression without the need for introduction of exogenous nucleic acid (such as by transduction or transfection). This is in contrast to conventional methods for expanding NK cells with increased CD16 expression, which require expression of an exogenous CD16 transgene to achieve even minimal increases in CD16 expression. Alternatively or in addition, another benefit of the CD16⁺ NK cells of the invention is that an NK cell population with an increased proportion CD16⁺ NK cells can be obtained with no or reduced purification and/or concentration compared with conventionally produced expanded NK cell populations. As such, conventionally produced expanded NK cell populations differ from those according to the invention, even if NK cells within said populations are manipulated to increase CD16 expression, and/or said conventionally produced NK cell population is manipulated to select and/or concentrate CD16⁺ NK cells, because (i) the CD16⁺ NK cells within such conventional populations will have been genetically altered and comprise exogenous nucleic acid, which is associated with disadvantages from a clinical/GMP perspective; and/or (ii) when extensive purification and/or concentration of CD16⁺ NK cells is required, this can result in damage to the purified and/or concentrated NK cells.

Accordingly, the invention provides a population of NK cells comprising an increased number of CD16⁺ NK cells (also referred to herein as a CD16⁺ NK cell population), wherein the CD16⁺ NK cells have not been contacted with an exogenous nucleic acid (typically a nucleic acid encoding for CD16). Typically, a population of NK cells comprising an increased number of CD16⁺ NK cells according to the invention may comprise CD16⁺ NK cells that do not comprise exogenous nucleic acid encoding for CD16. In view of the potential clinical application of the NK cells of the invention, a population of NK cells comprising an increased number of CD16⁺ NK cells according to the invention may comprise CD16⁺ NK cells that do not comprise any exogenous nucleic acid. The term “exogenous nucleic acid” encompasses a naked nucleic acid (e.g. a plasmid), or a viral vector (e.g. AAV or lentiviral vector) used to introduce a nucleic acid of interest (e.g. a nucleic acid encoding CD16) into NK cells. Alternatively or in addition, the invention provides a population of NK cells comprising an increased number of CD16⁺ NK cells (also referred to herein as a CD16⁺ NK cell population), wherein the CD16⁺ NK cells have been subjected to no or reduced purification and/or concentration compared to CD16⁺ NK cells that have been produced by conventional means, including means comprising the use of exogenous nucleic acid encoding for CD16.

As described herein, CD16⁺ NK cells are more effective at mediating ADCC, as it is binding of CD16 to the Fc portion of antibodies that triggers degranulation of NK cells and hence ADCC. Accordingly, by providing expanded NK cell populations with increased numbers of CD16⁺ NK cells, the invention provides CD16⁺ NK cells and expanded NK cell populations which exhibit increased ADCC, i.e. increased ADCC activity. This increase in ADCC can be compared with a suitable reference or control NK cell or NK cell population. A control method may be any standard method known in the art for producing NK cell populations. For example, a control method may use conventional adoptive transfer techniques, rather than a method according to the present invention. One example of suitable control NK cells or NK cell populations is an NK cell or NK cell population produced by a corresponding method in which the HPCs are not cultured in a medium which does not induce differentiation of the HPCs to NK cells, or wherein the HPCs are cultured in a medium which does not induce differentiation of the HPCs to NK cells for less than 2 days. In other words, one example of suitable control NK cells or NK cell populations is an NK cell or NK cell population produced by a corresponding method in which step (a) as described herein is omitted. NK cells and NK cell populations produced by such control/standard methods may be used as control cells and populations as described herein.

An expanded population of NK cells according to the invention or the CD16⁺ NK cells within said population may exhibit at least 25% greater ADCC, such as at least 30% greater ADCC, at least 40% greater ADCC, at least 50% greater ADCC, at least 60% greater ADCC at least 70% greater ADCC, at least 80% greater ADCC, preferably at least 50% greater ADCC, more preferably at least 60% greater ADCC, still more preferably at least 70% greater ADCC, compared with a suitable reference or control NK cell or NK cell population.

### Expanded NK Cell Populations

As disclosed herein, the invention provides methods for generating an expanded population of NK cells comprising an increased number of CD16⁺ NK cells (referred to interchangeably herein as an expanded CD16⁺ NK cell population, a CD16⁺ NK cell population, an NK cell population or an expanded NK cell population of the invention). Any of the disclosure herein in relation to CD16+ NK cells of the present invention may also be applied to a CD16⁺ NK cell population of the invention.

Accordingly, the present invention provides a CD16⁺ NK cell population. Typically a CD16+ NK cell population of the invention comprises iNK cells, mNK cells and/or cNK cells, or a combination thereof. Said population may comprise HPCs, such as HSCs, CLP cells and/or NKPs, or a combination thereof, although the numbers of such cells is typically low relative to the number of NK cells, as the majority of these HPCs have differentiated into NK cells in the population. Said population may comprise other immune and/or non-immune cells. Again, the number of any such cells is typically low relative to the number of NK cells present in the population.

As a non-limiting example, at least 60%, at least 70%, at least 80%, at least 85%, at least 90%, at least 91%, at least 92%, at least 93%, at least 94%, at least 95%, at least 96%, at least 97%, at least 98%, at least 99% or more, up to 100% of the cells of a CD16⁺ NK cell population of the invention may be NK cells. Typically at least 80%, preferably at least 85%, more preferably at least 90%, or even more preferably at least 95% of the cells of a CD16⁺ NK cell population of the invention are NK cells.

At least 60%, at least 70%, at least 80%, at least 85%, at least 90%, at least 91%, at least 92%, at least 93%, at least 94%, at least 95%, at least 96%, at least 97%, at least 98%, at least 99% or more, up to 100% of the cells of a CD16⁺ NK cell population of the invention are mature NK cells (i.e. mNK cells and/or cNK cells). Preferably at least 80%, more preferably at least 85%, even more preferably at least 90%, even more preferably at least 95%, even more preferably at least 98% or more of the cells of a CD16⁺ NK cell population of the invention are mature NK cells.

The number of HPCs (including HSCs, CLP cells and/or NKPs) may be less than 40%, less than 30%, less than 25%, less than 20%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1% of the cells of the CD16⁺ NK cell population. Typically the number of HPCs (including HSCs, CLP cells and/or NKPs) is less than 20%, preferably less than 15%, more preferably less than 10%, even more preferably less than 5%, even more preferably less than 2% or less of the cells of the CD16⁺ NK cell population.

The number of other immune and/or non-immune cells may be less than 40%, less than 30%, less than 25%, less than 20%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1% of the cells of the CD16⁺ NK cell population. Typically the number of other immune and/or non-immune cells is less than 20%, preferably less than 15%, more preferably less than 10%, even more preferably less than 5% of the cells, even more preferably less than 2%, or less of the CD16⁺ NK cell population.

As described herein, the CD16⁺ NK cell populations made by the methods of the present invention offer several advantages over NK cell populations made by conventional methods. In particular, the methods of the present invention enable the production of expanded populations with greater number of CD16⁺ NK cells compared with conventional methods. Further, a greater proportion of the NK cells in a population of the invention are CD16⁺ and hence functional, preferably fully functional, compared with populations obtained by conventional methods, in which a large number of the NK cells are “exhausted”.

As used herein, the term “exhausted” in the context of NK cells means that an NK cell or expanded NK cell population has lost at least some of its effector functions, such as cytotoxic function, cytokine production and/or ADCC. Thus, an exhausted NK cell or expanded NK cell population may exhibit impaired survival, impaired cytotoxic function, altered or impaired cytokine production and/or impaired ADCC. For example, an exhausted NK cell or exhausted NK cell population may exhibit at least a 50% reduction in one of its effector functions. For example, at least a 50% reduction in cytokine secretion, at least a 50% reduction in ADCC and/or at least 50% reduction in cytotoxic activity. These values may be quantified relative to any appropriate control as defined herein. Any appropriate technique can be used to determine effector function, and hence to quantify and reduction therein. Suitable techniques are known in the art. Alternatively and/or in addition, exhausted NK cells may exhibit altered marker expression, such as an increase in the expression of one or more inhibitory receptor (as described herein) and/or a decrease in the expression of one or more activatory receptor (as described herein). Increased expression of NKG2A and/or Tim3 may be used as a marker for NK cell exhaustion. Again, the expression of these markers may be quantified relative to any appropriate control as defined herein.

In contrast, the terms “functional” and “fully functional” in the context of NK cells means that an NK cell or expanded NK cell population has all of the expected effector functions when responding to a given immune challenge. Thus, a (fully) functional NK cell or expanded NK cell population will typically exhibit cytotoxic function, cytokine production and/or ADCC as would be observed in vivo when NK cells are activated in response to an immune challenge, and will typically exhibit enhanced survival compared with NK cells produced using conventional methods. Alternatively and/or in addition, (fully) functional NK cells may exhibit altered marker expression, such as an increase in the expression of one or more activatory receptor (as described herein) and/or a decrease in the expression of one or more inhibitory receptor (as described herein). As a non-limiting example, a functional (mature) human NK cell may be CD56⁺ and/or CD45⁺, preferably both CD56⁺ and CD45⁺.

In addition, and as described herein, the (fully) functional NK cells of the invention are CD16⁺, and the CD16⁺ NK cell populations of the invention comprised an increased number of CD16⁺ NK cells compared with any appropriate control.

Alternatively or in addition to detecting and/or quantifying CD16 expression to determine the functionality of NK cells, the cytotoxicity of NK cells may be determined using a degranulation assay in NK cells co-incubated with ‘target cells’. A degranulation assay involves analysing the expression of CD107a within the NK cell population. The amount of CD107a correlates with cytokine secretion and NK cell-mediated lysis of target cells. NK cells can also be analysed for the expression of Interferon-γ (IFN-γ), which is the main cytokine secreted when functional NK cells are activated. NK cells that are functional should express similar or higher CD107a as well as IFN-γ when compared to a control.

Further alternatively or additionally, the cytotoxicity of the NK cells may be determined using flow cytometry to quantify cell death in a target cell population. Said target cell population may optionally be pre-labelled with fluorescent marker. The use of flow cytometry to quantify cell death in a target cell population may be a preferred means of determining the cytotoxicity of the NK cells.

Any increase in CD16⁺ NK cell number/functionality in a CD16⁺ NK cell population made by a method of the present invention may be compared with the NK cell number/function of an NK cell population obtained from a control method as described herein.

As a CD16⁺ NK cell population of the present invention comprises significantly fewer exhausted NK cells and/or CD16⁻ NK cells compared to conventionally prepared NK cell populations, but instead contains a higher proportion of fully functional CD16⁺ NK cells, this advantageously allows the use of smaller numbers of cells to treat patients.

As described herein, the methods of the invention produce NK cell populations with a higher proportion of (fully) functional NK cells compared with conventional methods, which produce populations with large numbers of “exhausted” NK cells. Typically, in a CD16⁺ NK cell population of the invention at least 40%, at least 50%, at least 60%, at least 70%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or more, up to 100% of the NK cells of an expanded NK cell population of the invention are (fully) functional. Typically at least 80%, preferably at least 85%, more preferably at least 90%, even more preferably at least 95%, even more preferably at least 98% or more of the NK cells of an expanded NK cell population of the invention are fully functional, according to any definition (e.g. marker and/or effector function definition) herein. The functionality of the NK cells of the invention preferably correlates with CD16 expression. By way of non-limiting example, at least 40% of the NK cells in a CD16⁺ NK cell population may be functional and at least 40% of the NK cells may be CD16⁺. Alternatively, the proportion of functional NK cells may be greater than the proportion of CD16⁺ NK cells. By way of non-limiting example, at least 60% of the NK cells in a CD16⁺ NK cell population may be functional and at least 40% of the NK cells may be CD16⁺. By way of a further alternative, the proportion of CD16⁺ NK cells may be greater than the proportion of functional NK cells. By way of non-limiting example, at least 40% of the NK cells in a CD16⁺ NK cell population may be functional and at least 60% of the NK cells may be CD16⁺.

An expanded NK cell population of the invention may be produced by any of the methods disclosed herein. Typically a CD16⁺ NK cell population of the invention is produced by an ex vivo method as disclosed herein.

### Notch Ligand

The Notch signalling pathway is primarily associated with promoting T cell development and repressing concomitant B cell development. Mammals have four types of Notch receptor—Notch1, Notch2, Notch3 and Notch4, all of which are single-pass heterodimeric transmembrane protein.

Mammals have two types of canonical Notch ligands—Delta type and Jagged type, collectively known as DSL ligands. There are three delta-like ligands (DLLs), DLL1, DLL3 and DLL4 and two jagged (JAG) ligands, JAG1 and JAG2. DLL and JAG ligands typically comprise the following domains: a module at the N-terminus of Notch ligand (MNNL) domain and a Delta/Serrate/Lag-2 (DSL) domain, together with a number of EGF repeats. DLL3 comprises six EGF repeats. DLL1 and DLL4 comprise eight EGF repeats. JAG1 and JAG2 comprise 16 EGF repeats. There are also numerous non-canonical ligands, which may be membrane-bound or secreted.

Unless explicitly stated herein, a reference herein to a Notch ligand is a reference to any Notch ligand, such as a ligand of Notch1, Notch2, Notch3 and/or Notch 4, preferably a ligand of at least Notch1. The protein sequence of human Notch1 is given in SEQ ID NO: 10 (GenBank Accession No. CR457221, version CR457221.1), with the corresponding cDNA sequence in SEQ ID NO: 9 (GenBank Accession No. CR457221, version CR457221.1). Typically the Notch ligand of use in the present invention is a canonical Notch ligand. In some preferred embodiments, the Notch ligand is a DLL, more preferably DLL4. The protein sequence of human DLL4 is given in SEQ ID NO: 8 (GenBank Accession No. AF253468, version AF253468.1), with the corresponding mRNA sequence in SEQ ID NO: 7 (GenBank Accession No. AF253468, version AF253468.1).

A reference herein to a Notch ligand also embraces fragments thereof, provided said fragment retains the Notch-binding and activatory activity of the Notch ligand from which it is derived. Fragments of Notch ligands suitable for use in the present invention have previously been described by the present inventors (see WO2018/178666, which is herein incorporated by reference in its entirety, particularly pages 15 and 16 and the Examples). Preferred examples of Notch ligand fragments include Notch ligand (N-EGF1) and Notch ligand (N-EGF2), such as DLL4 (N-EGF1) and DLL4 (N-EGF2), particularly DLL4.

Alternatively or in addition, a Notch ligand, fragment thereof, or molecule that mimics the effect (e.g. function/activity) of a Notch ligand, such as DLL4 may comprise modifications, such as amino acid mutations which alter, typically increase, the affinity of the ligand/fragment/mimetic for its Notch receptor. Techniques for identifying such modifications are known in the art. For example, amino acids which increase the affinity of a Notch ligand/fragment/mimetic can be identified using yeast surface display. Again, such modifications have previously been described by the present inventors (see WO2018/178666, which is herein incorporated by reference in its entirety, particularly page 16). In some preferred embodiments, the DLL4 ligand of the invention, a fragment or mimetic thereof comprises the amino acid substitutions, G28S, F107L and L206P, more preferably G28S, F107L, N1181, 1143F, H194Y, L206P and/or K215E.

As a further non-limiting example, a functional fragment of DLL4 comprises at least residues 65 to 114 and 179 to 219 of full-length DLL4, preferably held in the correct conformation to allow interaction with the Notch ligand.

In addition, the invention encompasses the use of molecules that would mimic the effect (e.g. activity/function) of a Notch ligand (also referred to herein as mimetics). For example, the use of peptides, stapled peptides, peptoids and peptidomimetics that would mimic the effect of the desired Notch ligand (such as DLL4) is embraced by the present invention. Peptidomimetics may have advantages over peptides in terms of stability and bioavailability associated with a natural peptide. Peptidomimetics can have main- or side-chain modifications of the parent peptide designed for biological function. Examples of classes of peptidomimetics include, but are not limited to, peptoids and β-peptides, as well as peptides incorporating D-amino acids.

Methods for producing synthetic peptides and peptidomimetics (such as peptoids) are known in the art, as are the sequences of canonical and non-canonical Notch ligands. Thus, it would be routine for one of skill in the art to produce suitable molecules which mimic the effect of a desired Notch ligand using known techniques and based on the known Notch ligand sequences. As a non-limiting example, peptidomimetics may be designed to interact with key residues of Notch (e.g. Notch1) that are known to be involved in binding to DLL4, such as one or more of residues 415 (E415), 418 (L418), 420 (A420), 421 (N421), 422 (P422), 424 (E424), 425 (H425), 436 (F436), 447 (P447), 448 (R448), 450 (E450), 452 (D452), 469 (D469), 477 (1477), 480 (P480) of Notch (Notch1), or any combination thereof.

The methods of the invention may encompass the use of any Notch ligand or fragment thereof which is capable of increasing NK cell production or molecule which mimics the effects thereof. A Notch ligand or fragment thereof may be used in a method of the invention, wherein said method does not comprise the use of a compound which inhibits REV-ERB activity as described herein. A Notch ligand or fragment thereof may be used in a method of the invention which further comprises the use of a compound which inhibits REV-ERB activity, or a compound which results in the alteration of post-translational modification of E4bp4, and hence an increase in E4bp4 activity as disclosed herein. In particular, a Notch ligand or fragment thereof may be used in a method of the invention which further comprises the use of a compound which inhibits REV-ERB activity, or a compound which results in the alteration of post-translational modification of E4bp4, and hence an increase in E4bp4 activity, wherein the Notch ligand or fragment thereof synergistically with a compound of the invention which inhibits REV-ERB activity as disclosed herein, or a compound which results in the alteration of post-translational modification of E4bp4, and hence an increase in E4bp4 activity.

The present inventors have previously shown that E4bp4 directly binds to the regulatory region of the Notch1 gene in vivo and so could enhance the transcriptional regulation of Notch, and that Notch1 expression E4bp4^{-/-} mice is significantly reduced. Following on from this, the present inventors found that short-term exposure of Notch ligands to murine HSCs and very early progenitors can promote NK cell development, even in the absence of the critical transcription factor E4bp4. Further, the present inventors have shown that the Notch ligand Delta-like ligand 4 (DLL4) is particularly effective in stimulating the expansion of NK cells.

Accordingly, the present invention relates to increasing the number of CD16⁺ NK cells in an expanded NK population, or the expansion of CD16⁺ NK cells, by exposure of the HPCs to a Notch ligand in a method which comprises a step of culturing HPCs in a medium that does not induce the differentiation of the HPCs, particularly which does not induce the differentiation of the HPCs to NK cells. Said method may comprise exposing the HPCs to a Notch ligand as part of the step of culturing HPCs in a medium that does not induce the differentiation of the HPCs, particularly which does not induce the differentiation of the HPCs to NK cells. Said methods may further comprise the use of a compound which inhibits the action of REV-ERB as described herein. In said methods, the compound which inhibits the action of REV-ERB may be used before the HPCs are differentiated to produce NK cells or during the differentiation of the HPCs to NK cells. Preferably, the compound which inhibits the action of REV-ERB may be used before the HPCs are differentiated to produce NK cells.

In ex vivo or in vitro methods of the invention, exposure of the HPCs to the Notch ligand can comprise a step of culturing the HPCs in the presence of a Notch ligand, such as in step (a) of the methods described herein. For in vivo methods, this may comprise administering the compound together with a Notch ligand. In preferred embodiments, the Notch ligand is DLL4, or a fragment or variant thereof which retains the function of DLL4. Any and all disclosure herein in relation to the use of a Notch ligand applies equally and without reservation to the preferred Notch ligand DLL4 and to fragments/variants/mimetics thereof.

Variant Notch ligands and/or fragments/mimetics thereof may be used according to the invention. The variant Notch ligands/fragments/mimetics of the invention typically at least retain the activity of the corresponding Notch ligands/fragments/mimetics of the invention. Thus, for example, the variant DLL4 ligands or fragments thereof of the invention retain the ability of the corresponding DLL4 molecules to bind to Notch1, and/or to enhance NK cell production. In some embodiments, the variant DLL4 ligands/fragments/mimetics have greater activity than the corresponding unmodified DLL4 ligand/fragment/mimetic. Such variants have previously been described by the present inventors (see WO2018/178666, which is herein incorporated by reference in its entirety, particularly pages 17 and 18). By way of non-limiting example, a Notch ligand/fragment/mimetic/variant thereof (e.g., a DLL4 ligands/fragments/mimetics/variant thereof) may have a K_{D} value for binding to Notch1 of less than 1 μM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM or less, preferably less than 500 nM, less than 400 nM, less than 300 nM or less. In some embodiments, a variant Notch ligand/fragment/mimetic (e.g., a variant DLL4 ligands/fragments/mimetics) can increase the number of NK cells, or give rise to an increase in NK cell production, of at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 3 fold or more relative to the corresponding unmodified Notch ligand/fragment/mimetic. The variant Notch ligand/fragment/mimetic (e.g., variant DLL4 ligands/fragments/mimetics) may increase number of NK cells by at least 10%, at least 20%, at least 30%, at least 40%, at least 50%, at least 60, at least 70%, at least 80%, at least 85%, at least 90%, at least 95%, at least 100%, at least 150%, at least 200%, at least 300% or more compared with the corresponding unmodified DLL4 ligand/fragment/mimetic.

The Notch ligands/fragments/mimetics of the invention may be labelled (or tagged). Any appropriate label may be used. Suitable labels are known in the art.

### E4bp4

E4bp4 (also known as Nfil3) is a basic leucine zipper protein transcription factor which is involved in the regulation of IL-3 expression, and is involved in the coordinating the circadian clock. The mRNA sequence of the human E4bp4 gene is given in SEQ ID NO: 1 (Genbank Accession No. X64318, version X64318.1), with the corresponding amino acid sequence in SEQ ID NO: 2. As shown in FIG. 1, E4bp4 is expressed in CLPs and is critical in the production of NK cells from blood stem cell progenitors. Mice with the E4bp4 gene deleted do not have functional NK cells, but have normal numbers of T and B cells. In contrast, overexpression of E4bp4 in HSCs in vitro increases NK cell production. Thus, E4bp4 is a lineage commitment factor, controlling the development of NKPs from HSCs (FIG. 1). E4bp4's critical function in NK cells is specific to the early stages of the developmental pathway, as specific ablation of E4bp4 in peripheral mNK cells does not affect NK cell number or response to cytomegalovirus infection. In addition E4bp4 regulates other transcription factors that are essential in NK cell development, such as Id2 and Eomes.

Although IL-7 and IL-15 have been shown to regulate E4bp4 expression, generally very little is known about how either extrinsic or intrinsic stimuli influence E4bp4. Transcription factors such as E4bp4 can be hard to target because of their structure and function. For example, they usually lack enzymatic activity or cofactor binding sites. However, the present inventors have previously demonstrated that E4bp4 expression can be increased using a compound which inhibits the activity of REV-ERB (see WO2018/158587, particularly the Examples thereof, which is herein incorporated by reference in its entirety). Further, the present inventors have demonstrated that the use of a REV-ERB inhibitor to increase E4bp4 expression results in an increase in NK cell number. Without wishing to be bound by theory, REV-ERB binds to porphyrin heme, and it is this characteristic that is believed to make REV-ERB a druggable target (see below). As such, the inventors have previously shown that by targeting REV-ERB and inhibiting its activity, it is possible to increase E4bp4 expression and hence increase NK cell number. Accordingly, compounds which inhibit the action of REV-ERB, and hence increase E4bp4 expression, and NK cell number can be used in the methods of the invention to increase the number of CD16⁺ NK cells.

### Increase in E4bp4 Expression

As described herein, the present invention provides ex vivo methods for producing expanded CD16⁺ NK cell populations, and methods of increasing the number of CD16⁺ NK cells in an expanded NK cell population, as well as therapeutic methods and applications for increasing NK cell number in a patient in need thereof. As disclosed herein, said methods and applications may involve the use of a compound which inhibits the action of REV-ERB. Typically said compounds act by increasing E4bp4 expression.

An increase in E4bp4 expression may be measured relative to a control. Thus, the expression of E4bp4 in a sample of HPCs, an expanded NK cell population or in a sample obtained from an individual/patient to be treated according to the invention may be compared with the expression of E4bp4 in a control. Expression may be quantified in terms of gene and/or protein expression, and may be compared with expression of a control (e.g. housekeeping gene or protein). The actual amount of the E4bp4 gene, mRNA transcript and/or protein, such as the mass, molar amount, concentration or molarity of the E4bp4 gene, mRNA transcript and/or protein, or the number of mRNA molecules per cell in a sample of HPCs, an expanded NK cell population or in a sample obtained from an individual/patient to be treated according to the invention and the control may be assessed and compared with the corresponding value from the control. Alternatively, the expression of the E4bp4 gene and/or protein in a sample of HPCs, an expanded CD16⁺ NK cell population or in a sample obtained from an individual/patient to be treated according to the invention may be compared with that of the control without quantifying the mass, molar amount, concentration or molarity of the one or more gene and/or protein.

The control may be as described herein. In the context of E4bp4 expression, the control may be an equivalent population or sample in which no increase in E4bp4 expression has been effected. As a non-limiting example, in the case where an individual/patient is treated with a compound that inhibits REV-ERB activity in order to increase E4bp4 expression, a suitable control would be a different individual to which the compound has not been administered or the same individual prior to administration of the compound. Conventional methods for the ex vivo expansion of NK cells, including known methods may be considered control methods according to the present invention.

In the context of the present invention, a reference to increasing E4bp4 expression may be understood to mean that, the expression of E4bp4 is increased by at least 10%, at least 20%, at least 30%, at least 40%, at least 50%, at least 60, at least 70%, at least 80%, at least 85%, at least 90%, at least 95%, at least 100%, at least 150%, at least 200% compared with the control. Typically E4bp4 expression is increased by at least 50%, preferably at least 70%, more preferably at least 80%, even more preferably at least 90% or more compared with the control.

A reference to increasing E4bp4 expression may be understood to mean that, the expression of E4bp4 is increased by at least 1.5-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, at least 4-fold, at least 5-fold, at least 6-fold, at least 7-fold, at least 8-fold, at least 9-fold, at least 10-fold or more relative to a control. Typically E4bp4 gene expression is increased by at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, or more compared with the control. Typically E4bp4 protein expression is increased by at least 2-fold, at least 3-fold, preferably at least 5-fold, more preferably at least 6-fold or more compared with the control.

The expression of the E4bp4 gene and/or protein according to the invention may be determined by quantitative and/or qualitative analysis. Typically, gene expression may be expressed in terms of mRNA levels.

The expression level of the E4bp4 gene and/or protein according to the invention encompasses the mass of the E4bp4 mRNA transcript and/or protein, the molar amount of the E4bp4 gene, mRNA transcript and/or protein, the concentration of the E4bp4 gene and/or protein and the molarity of the E4bp4 gene and/or protein. This expression level may be given in any appropriate units. For example, the concentration of the E4bp4 gene and/or protein may be given in pg/ml, ng/ml or μg/ml.

The expression level of the E4bp4 gene and/or protein according to the invention may be measured directly or indirectly.

The relative expression of the E4bp4 gene and/or protein according to the invention relative to a control may be determined using any appropriate technique. Suitable standard techniques are known in the art, for example Western blotting, enzyme-linked immunosorbent assays (ELISAs) and RT-qPCR.

The expression level of the E4bp4 gene and/or protein may be increased compared with a control for at least 6 hours, at least 12 hours, at least 24 hours, at least 30 hours, at least 36 hours, at least 42 hours, at least 48 hours, at least 54 hours, at least 60 hours, at least 72 hours, at least 4 days, at least 5 days, at least 6 days, at least 1 week. Preferably, the expression level of the E4bp4 gene and/or protein is increased for at least 12 to 72 hours. Typically this is assessed relative to the last administration of the compound which inhibits REV-ERB activity.

The expression level of the E4bp4 gene and/or protein may be increased compared with a control for at least one, at least two, at least three, at least four, at least five, at least ten, at least 20, at least 30, at least 40 or more passages of the NK cell precursors in culture. The expression level of the E4bp4 gene and/or protein may be altered indefinitely.

### REV-ERB

REV-ERB proteins are members of the nuclear receptor family of intracellular transcription factors. The mRNA sequence of the human REV-ERBα gene (Nr1d1) is given in SEQ ID NO: 3, and the amino acid sequence in SEQ ID NO: 4 (Genbank Accession No. NM_021724, version NM_021724.4). The mRNA sequence of the human REV-ERBβ gene (Nr1d2) is given in SEQ ID NO: 5 (Genbank Accession No. AB307693, version AB307693.1), and the corresponding amino acid sequence in SEQ ID NO: 6. REV-ERB regulates the circadian clock, and has also been implicated in the regulation of cartilage breakdown.

The present inventors have previously demonstrated that inhibition of REV-ERB activity is sufficient to elicit a significant increase in E4bp4 expression, and that this in turn brings about an expansion of NK cells, resulting in an increase in NK cell number (see WO2018/158587, particularly the Examples, which is herein incorporated by reference in its entirety). Inhibition of REV-ERB activity can bring about an increase in NK cell number, and that typically the resulting NK cells are (fully) functional as defined herein. The effect of REV-ERB inhibition is mediated in an E4pb4-dependent manner. Without wishing to be bound by theory, it is believed that inhibition of REV-ERB activity results in an increase in E4bp4 expression (E4bp4 expression is normally repressed by REV-ERB), and that the E4bp4 acts to stimulate the production of NK cells (as shown in FIG. 1). In particular, the present inventors have previously demonstrated that a class of small molecules, and particularly SR8278 (1,2,3,4-Tetrahydro-2-[[5-(methylthio)-2-thienyl]carbonyl]-3-isoquinolinecarboxylic acid ethyl ester, CAS No: 1254944-66-5), capable of binding to the porphyrin heme moiety of REV-ERB, resulting in inhibition of REV-ERB activity and an increase in NK cell number. The present inventors have also previously developed novel small molecules which are effective in inhibiting REV-ERB, and particularly which are more effective inhibitors of REV-ERB than SR8278. These improved REV-ERB antagonists are described in WO2020/002911, particularly the Examples, which is herein incorporated by reference in its entirety. In particular, compounds 11 and 7 of WO2020/002911 are encompassed by the present invention.

### Inhibition of REV-ERB Activity

In some embodiments, the present invention relates to the use of compounds to inhibit the action of REV-ERB, i.e. compounds which inhibit REV-ERB activity. REV-REB activity may be inhibited by any appropriate means. Suitable standard techniques are known in the art. Inhibition may take place via any suitable mechanism, depending for example on the nature (see below) of the compound used, e.g. steric interference in any direct or indirect interaction or inhibition of REV-ERB. In the context of the present invention a REV-ERB inhibitor (interchangeably referred to herein as a REV-ERB antagonist) is any compound which inhibits, decreases, suppresses or ablates the action of REV-ERB, whether in part or completely.

A decrease in REV-ERB activity may be measured relative to a control. Thus, the activity of REV-ERB in a sample of NK precursor or progenitor cells, an expanded CD16⁺ NK cell population or in a sample obtained from an individual/patient to be treated according to the invention may be compared with the activity of REV-ERB in a control. Activity may be quantified in any appropriate terms, for example binding of REV-ERB to the E4bp4 gene, or in terms of E4bp4 expression as defined herein. Any appropriate technique or method may be used for quantifying REV-ERB activity. Suitable techniques are known in the art, for example luciferase assays for quantifying expression of a reporter gene.

The control may be as described herein. In the context of REV-ERB activity, the control may be an equivalent population or sample in which no REV-ERB inhibitory compound has been added, for example a sample obtained from a different individual to which the compound has not been administered, or the same individual the prior to administration of the compound. Conventional methods for the ex vivo expansion of NK cells, including known methods may be considered control methods according to the present invention.

In the context of the present invention, a reference to inhibiting REV-ERB activity may be understood to mean that, the activity of REV-ERB is decreased by at least 10%, at least 20%, at least 30%, at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, at least 99%, up to total (100%) inhibition of REV-ERB activity, as compared with the control. Typically REV-ERB activity is decreased by at least 50%, preferably at least 70%, more preferably at least 80%, more preferably at least 90%, even more preferably at least 95% or more compared with the control.

The activity of REV-ERB may be determined by quantitative and/or qualitative analysis, and may be measured directly or indirectly.

The activity of REV-ERB relative to a control may be determined using any appropriate technique. Suitable standard techniques are known in the art, such as by quantifying E4bp4 expression, and/or luciferase assays.

The activity of REV-ERB may be inhibited compared with a control for at least 6 hours, at least 12 hours, at least 24 hours, at least 30 hours, at least 36 hours, at least 42 hours, at least 48 hours, at least 54 hours, at least 60 hours, at least 72 hours, at least 4 days, at least 5 days, at least 6 days, at least 1 week. Preferably, the activity of REV-ERB is decreased for at least 12 to 72 hours. Typically this is assessed relative to the last administration of the compound which inhibits REV-ERB activity.

The activity of REV-ERB may be inhibited compared with a control for at least one, at least two, at least three, at least four, at least five, at least ten, at least 20, at least 30, at least 40 or more passages of the cells (either in vivo, or cultured ex vivo or in vitro). The activity of REV-ERB may be inhibited and/or the expression level of the E4bp4 gene and/or protein may be altered indefinitely.

In the context of the present invention any reference to inhibiting REV-ERB activity may be understood to mean inhibiting the activity of REV-ERBα and/or REV-ERBβ. In preferred embodiments, the activity of both REV-ERBα and REV-ERBβ is inhibited. Thus, the invention relates to compounds which inhibit REV-ERB activity, including compounds which inhibit REV-ERBα activity (i.e. REV-ERBα inhibitors, also referred to as REV-ERBα antagonists) and/or to compounds which inhibit REV-ERBβ activity (i.e. REV-ERBβ inhibitors, also referred to as REV-ERBβ antagonists). In preferred embodiments, the invention relates to compounds which inhibit the activity of both REV-ERBα and REV-ERBβ (i.e. REV-ERBα and REV-ERBβ inhibitors, also referred to as REV-ERBα and REV-ERBβ antagonists).

### REV-ERB Antagonists/Inhibitors

REV-ERB inhibitory compounds of the invention may be specific for REV-ERB. By specific, it will be understood that the compound binds to REV-ERBα and/or REV-ERBβ, with no significant cross-reactivity to any other molecule, particularly any other protein. For example, modulator that is specific for REV-ERBα and/or REV-ERBβ will show no significant cross-reactivity with human neutrophil elastase. Cross-reactivity may be assessed by any suitable method. Cross-reactivity of REV-ERBα and/or REV-ERBβ inhibitor with a molecule other than REV-ERBα and/or REV-ERBβ may be considered significant if the inhibitor binds to the other molecule at least 5%, 10%, 15%, 20%, 25%, 30%, 35%, 40%, 45%, 50%, 55%, 60%, 65%, 70%, 75%, 80%, 85%, 90% or 100% as strongly as it binds to REV-ERBα and/or REV-ERBβ. An inhibitor that is specific for REV-ERBα and/or REV-ERBβ may bind to another molecule such as human neutrophil elastase at less than 90%, 85%, 80%, 75%, 70%, 65%, 60%, 55%, 50%, 45%, 40%, 35%, 30%, 25% or 20% the strength that it binds to REV-ERBα and/or REV-ERBβ. Preferably, the inhibitor binds to the other molecule at less than 20%, less than 15%, less than 10% or less than 5%, less than 2% or less than 1% the strength that it binds to REV-ERBα and/or REV-ERBβ.

REV-ERB inhibitory compounds of the invention may have off-target effects. An off-target effect is activity against a target other than REV-ERB. Typically compounds with off-target effects are encompassed by the present invention if the activity against the non-REV-ERB target is not significant compared with the activity against REV-ERB. Whether an off-target effect is significant may depend on the intended use of the compound. As a non-limiting example, a compound which may exert an off-target effect on the central nervous system would not be significant for a compound used in an ex vivo method as disclosed herein, but may be significant (depending on the magnitude of the off-target effect) for an in vivo therapeutic indication as disclosed herein. The presence and magnitude of any potential off target effects can be readily assessed using standard methods known in the art.

Any suitable inhibitor may be used according to the invention, for example small molecules, PROTAC reagents, double stranded RNA (dsRNA), small interfering RNA (siRNA), small hairpin RNA (shRNA), microRNA, antisense (single stranded) RNA, peptides and peptidomimetics, antibodies, aptamers and ribozymes. Preferred inhibitors include small molecules and PROTAC reagents.

### Small Molecules

Small molecules may be used to inhibit REV-ERB activity as described herein. As defined herein, small molecules are low molecular weight compounds, typically organic compounds. Typically, a small molecule has a maximum molecule weight of 900 Da, allowing for rapid diffusion across cell membranes. In some embodiments, the maximum molecular weight of a small molecule is 500 Da. Typically a small molecule has a size in the order of 1 nm.

According to the present invention, small molecules may be able to exert an inhibitory effect on REV-ERB activity by binding to the porphyrin heme moiety of REV-ERB. Thus in some preferred embodiments, a compound that inhibits the action of REV-ERB according to the present invention is a compound which binds to the porphyrin heme moiety of REV-ERB, and hence inhibits the activity of REV-ERB. Alternatively, the small molecule may act via a different mechanism, for example, by binding to a non-heme portion of REV-ERB. Standard techniques are known in the art for the production of small molecules, which can then readily be tested for REV-ERB inhibitory activity as described herein

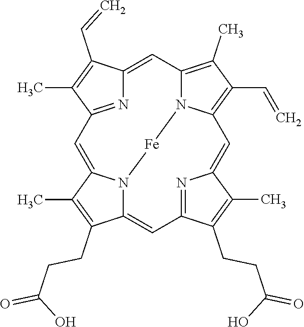

### Structure of Porphyrin Heme

The invention encompasses the use small molecule REV-ERB antagonists as described WO2018/158587 and WO2018/178666 (each of which is herein incorporated by reference in its entirety), as well as variants of said small molecule REV-ERB antagonists which retain the REV-ERB inhibitory function of the small molecule REV-ERB antagonist from which they are derived. Non-limiting examples include SR8278, GSK1362 and 4-[[[1-(2-fluorophenyl)cyclopentyl]amino]methyl]-2-[(4-methylpiperazin-1-yl)methyl]phenol (also referred to herein as ARN5187), ethyl 2-(5-methylfuran-2-carbonyl)-1,2,3,4-tetrahydroisoquinoline-3-carboxylate, 4-((4-chlorobenzyl)((5-nitrothiophen-2-yl)methyl)amino)-N-phenylpiperidine-1-carboxamide, 4-(((1-(4-fluorophenyl)cyclopentyl)amino)methyl)-2-((4-methylpiperazin-1-yl)methyl)phenol, 1-(2-fluorophenyl)-N-(3-((1-methylpiperidin-4-yl)methyl)benzyl)cyclopentan-1-amine and 1-(4-fluorophenyl)-N-(3-((1-methylpiperidin-4-yl)methyl)benzyl)cyclopentan-1-amine. Preferably, the invention encompasses the use of improved REV-ERB antagonists as described in WO2020/002911, particularly the Examples, which is herein incorporated by reference in its entirety. In particular, the use of compounds 11 and 7 of WO2020/002911 is encompassed by the present invention. Compounds 11 and 7 of WO2020/002911 have the following structures:

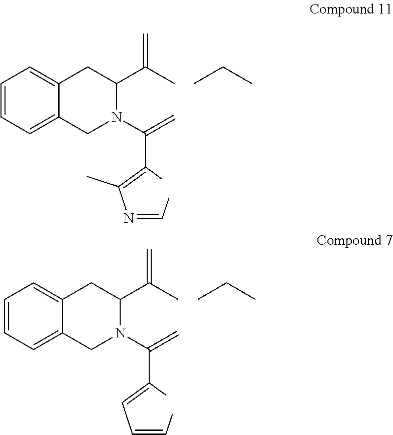

### PROTAC Reagents

Proteolysis targeting chimeras (also referred to as PROTACs or PROTAC reagents) may be used to inhibit REV-ERB activity as described herein. PROTACs are heterobifunctional small molecules that simultaneously bind a target protein and ubiquitin ligase, enabling ubiquitination and degradation of the target. In more detail, a PROTAC reagent typically comprises a ligand for the target protein (in the case of the present invention, REV-ERB) and a ligand for an E3 ligase recognition domain. Through the use of such a PROTAC, an E3 ligase is recruited to the PROTAC-bound REV-ERB, inducing ubiquitin transfer from the E3 ligase complex to the target protein (in the case of the present invention, REV-ERB). Once the PROTAC has induced a sufficient degree of ubiquitination of the target, it is then recognised and degraded by the proteasome.

As a non-limiting example, a PROTAC reagent may be produced by conjugating a ligand for an E3-ligase to a small molecule inhibitor as described herein (preferably SR8278) via a linker. In a preferred embodiment, a PROTAC reagent comprises a ligand for the E3 RING Cullin ligase von-Hippel Lindau protein (VHL) or cereblon—a part of a CRL4 E3 RING Cullin ligase complex, connected to a small molecule inhibitor of the invention via a linker. In some particularly preferred embodiments, the PROTAC reagent comprises a ligand for the E3 RING Cullin ligase von-Hippel Lindau protein (VHL) connected to SR8278, connected via a linker. In other particularly preferred embodiments, the PROTAC reagent comprises cereblon (a part of a CRL4 E3 RING Cullin ligase complex) and SR8278, connected via a linker.

Because of their mechanism of action, PROTAC reagents simply need any ligand for the target protein. The functional pharmacology of the ligand, in the absence of the linker and E3 ligase ligand, is unimportant. Therefore in some embodiments a REV-ERB inhibitory PROTAC reagent of the present invention may comprises a small molecule REV-ERB agonist as the ligand, such as GSK4112 (1,1-Dimethylethyl N-[(4-chlorophenyl)methyl]-N-[(5-nitro-2-thienyl)methyl])glycinate, SR6452).

### Double-Stranded RNA

Double-stranded RNA (dsRNA) molecules may be used to inhibit REV-ERB activity as described herein. dsRNA molecules may be used in RNAi to inhibit REV-ERB activity.

Using known techniques and based on a knowledge of the sequence of REV-ERB, dsRNA molecules can be designed to antagonise REV-ERB by sequence homology-based targeting of the corresponding RNA sequence. Such dsRNAs will typically be small interfering RNAs (siRNAs), small hairpin RNAs (shRNAs), or micro-RNAs (miRNAs). The sequence of such dsRNAs will comprise a portion that corresponds with that of a portion of the mRNA encoding REV-ERB. This portion will usually be 100% complementary to the target portion within the mRNA transcribed from the REV-ERB gene, but lower levels of complementarity (e.g. 90% or more or 95% or more) may also be used. Typically the % complementarity is determined over a length of contiguous nucleic acid residues. A dsRNA molecule of the invention may, for example, have at least 80% complementarity to the target portion within the mRNA transcribed from the REV-ERB gene measured over at least 10, at least 20, at least 30, at least 40, at least 50, at least 60, at least 70, at least 80, at least 90, or more nucleic acid residues, up to the dsRNA molecule having at least 80% complementarity the mRNA transcribed from the REV-ERB gene of the invention over the entire length of the dsRNA molecule.

In a preferred embodiment, the dsRNA is a shRNA. ShRNA can be delivered to NK cell precursors by any appropriate means. Suitable techniques are known in the art and include the use of plasmid, viral and bacterial vectors to deliver the shRNA. Typically, the shRNA is delivered using a viral vector delivery system. In a preferred embodiment, the viral vector is a lentiviral vector.

Generally, once the shRNA has been delivered to an NK precursor cell, it is then transcribed in the nucleus and processed. The resulting pre-shRNA is exported from the nucleus and then processed by dicer and loaded into the RNA-induced silencing complex (RISC). The sense (passenger) strand is degraded. The antisense (guide) strand directs RISC to mRNA that has a complementary sequence. In the case of perfect complementarity, RISC cleaves the mRNA. In the case of imperfect complementarity, RISC represses translation of the mRNA. In both of these cases, the shRNA leads to target gene silencing.

A variant sequence may have at least 80% sequence identity to an shRNA sequence of the invention, measured over any appropriate length of sequence. Typically the % sequence identity is determined over a length of contiguous nucleic acid or amino acid residues. A variant sequence of the invention may, for example, have at least 80% sequence identity to a sequence of the invention measured over at least 10, at least 20, at least 30, at least 40, at least 50, at least 60, at least 70, at least 80, at least 90, or more nucleic acid or amino acid residues.

For example, a variant shRNA molecule of the invention may have at least 80% sequence identity with an shRNA molecule of the invention measured over at least 10, at least 20, at least 30, at least 40, at least 50, at least 60 or more nucleic acid residues, up to the variant shRNA molecule having at least 80% sequence identity with the shRNA molecule of the invention over the entire length of the variant shRNA molecule.

### Antisense RNA

Single-stranded DNA (ssDNA) molecules, also known as antisense RNA, may be used to inhibit REV-ERB activity as described herein.

Using known techniques and based on a knowledge of the sequence of the REV-ERB gene, antisense RNA molecules can be designed to antagonise the REV-ERB gene by sequence homology-based targeting of the corresponding RNA. The sequence of such antisense will comprise a portion that corresponds with that of a portion of the mRNA transcribed from the REV-ERB gene. This portion will usually be 100% complementary to the target portion within the transcribed mRNA but lower levels of complementarity (e.g. 90% or more or 95% or more) may also be used.

### Aptamers

Aptamers are generally nucleic acid molecules that bind a specific target molecule. Aptamers can be engineered completely in vitro, are readily produced by chemical synthesis, possess desirable storage properties, and elicit little or no immunogenicity in therapeutic applications. These characteristics make them particularly useful in pharmaceutical and therapeutic utilities.

As used herein, “aptamer” refers in general to a single or double stranded oligonucleotide or a mixture of such oligonucleotides, wherein the oligonucleotide or mixture is capable of binding specifically to a target. Oligonucleotide aptamers will be discussed here, but the skilled reader will appreciate that other aptamers having equivalent binding characteristics can also be used, such as peptide aptamers.

In general, aptamers may comprise oligonucleotides that are at least 5, at least 10 or at least nucleotides in length. Aptamers may comprise sequences that are up to 40, up to 60 or up to 100 or more nucleotides in length. For example, aptamers may be from 5 to 100 nucleotides, from 10 to nucleotides, or from 15 to 40 nucleotides in length. Where possible, aptamers of shorter length are preferred as these will often lead to less interference by other molecules or materials.

Aptamers may be generated using routine methods such as the Systematic Evolution of Ligands by Exponential enrichment (SELEX) procedure. SELEX is a method for the in vitro evolution of nucleic acid molecules with highly specific binding to target molecules. It is described in, for example, U.S. Pat. Nos. 5,654,151, 5,503,978, 5,567,588 and WO 96/38579, each of which is herein incorporated by reference in its entirety.

The SELEX method involves the selection of nucleic acid aptamers and in particular single stranded nucleic acids capable of binding to a desired target, from a collection of oligonucleotides. A collection of single-stranded nucleic acids (e.g., DNA, RNA, or variants thereof) is contacted with a target, under conditions favourable for binding, those nucleic acids which are bound to targets in the mixture are separated from those which do not bind, the nucleic acid-target complexes are dissociated, those nucleic acids which had bound to the target are amplified to yield a collection or library which is enriched in nucleic acids having the desired binding activity, and then this series of steps is repeated as necessary to produce a library of nucleic acids (aptamers) having specific binding affinity for the relevant target.

### Peptidomimetics

Peptidomimetics are compounds which mimic a natural peptide or protein with the ability to interact with the biological target and produce the same biological effect. Peptidomimetics may have advantages over peptides in terms of stability and bioavailability associated with a natural peptide. Peptidomimetics can have main- or side-chain modifications of the parent peptide designed for biological function. Examples of classes of peptidomimetics include, but are not limited to, peptoids and β-peptides, as well as peptides incorporating D-amino acids.

### Antibodies

Antibodies may be used to inhibit REV-ERB activity as described herein.

As used herein, the term antibody encompasses the use of a monoclonal antibody or polyclonal antibody, as well as the antigen-binding fragments of a monoclonal or polyclonal antibody, or a peptide which binds to REV-ERB with specificity. The antibody may be a Fab, F(ab′)2, Fv, scFv, Fd or dAb.

### Post-Translational Modification of E4bp4

The present inventors have previously shown that alteration of post-translational modification of E4bp4 can increase E4bp4 activity (see WO2018/178666, which is herein incorporated by reference in its entirety, particularly pages 33 to 36 and Examples 1 to 5). Furthermore, increasing E4bp4 activity by alteration of post-translational modification results in an increase in NK cell number (as defined herein).

Accordingly, methods of the present invention may comprise a step of contacting an haematopoietic progenitor cell (HPC) comprising sample obtained from an individual/patient with a compound which results in the alteration of post-translational modification of E4bp4, thereby causing an increase in E4bp4 activity. Thus, compounds which alter the post-translational modification of E4pb4 as described herein may be used in combination with the methods of the invention. This combination may further be used in combination with the use of a Notch ligand (e.g. DLL4) and/or a compound which inhibits REV-ERB activity as described herein.

Similarly, a compound which alters or affects the post-translational modification of E4bp4 may therefore be used according to the invention for increasing production of NK cells in a patient, wherein said compound increases E4bp4 activity, or for use in a method of treatment by increasing the number of NK cells in a patient in need thereof, together with the methods disclosed herein relating increasing CD16⁺ NK cell numbers by inclusion of a pre-differentiation step (a), the method disclosed herein relating to increasing CD16⁺ NK cell number comprising a step of increasing E4bp4 expression by decreasing REV-ERB activity and/or the methods disclosed herein relating to increasing CD16⁺ NK cell number by culturing HPCs in the presence of a Notch ligand.

Any of the disclosure herein in relation to methods of increasing NK cell number, methods of expanding CD16⁺ NK cells, optionally in the context of compounds which inhibit the action of REV-ERB, and/or Notch ligands, expanded NK cell populations produced by said methods and therapeutic indications relating to said compounds and populations applies inter alia to the disclosed methods of increasing E4bp4 activity to increase CD16⁺ NK cell number. As non-limiting examples, the feeder cell layers, growth factors and/or other culture conditions and diseases to be treated may be the same in relation to the post-translational modification aspects as for the other aspects (e.g. the REV-ERB inhibition and/or Notch ligand aspects) disclosed herein. The REV-ERB inhibitor compound, Notch ligand and/or E4bp4 post-translational modifier may be used simultaneously, separately or sequentially. When a compound which alters the post-translational modification of E4bp4 is used in combination with a compound which inhibits the action of REV-ERB, typically the sample is contacted with REV-ERB inhibitory compound before being contacted with the post-translational modifier. If a Notch ligand is also used, typically the E4bp4 post-translational modifier is used after the REV-ERB inhibitory compound and the Notch ligand; preferably the REV-ERB inhibitory compound are used together, or more preferably the REV-ERB inhibitory compound is used before the Notch ligand (as described herein).

### Types of Post-Translational Modification

Said method encompasses any alteration of post-translational modification which results in an increase in E4bp4 activity. Non-limiting examples of post-translation modification include phosphorylation, SUMOylation, the addition of a hydrophobic group (e.g. myristoylation, palmitoylation), addition of a cofactor, the addition of small chemical groups (e.g. acylation, alkylation, amidation, glycosylation), glycation, carbamylation, cabonylation, chemical modifications (e.g. deamidation) and/or structural changes. Typically alteration of post-translational modification according to the invention results in a reduction in phosphorylation at one or more phosphorylation site within wild-type (unmodified) E4bp4 and/or a reduction in SUMOylation at one or more SUMOylation site within wild-type (unmodified) E4bp4, or a combination thereof. As previously shown by the inventors (see WO2018/178666, which is herein incorporated by reference in its entirety, particularly pages 33 to 36 and Examples 1 to 5), wild-type (unmodified) E4bp4 is typically SUMOylated at one or more of residues K10, K116, K219, K337 and/or K394 or residues corresponding thereto, or any combination thereof. Typically wild-type (unmodified) E4bp4 is SUMOylated at least at residue K219 (or a corresponding residue). Alternatively or in addition, wild-type (unmodified) E4bp4 is typically phosphorylated at residues S286, S301 and S454, or residues corresponding thereto, or any combination thereof. Accordingly, in some embodiments, a compound which alters the post-translational modification of E4bp4 reduces, inhibits or ablates SUMOylation at residue K219 (or a residue corresponding thereto), and/or reduces, inhibits or ablates phosphorylation at residues S286, S301 and S454 (or corresponding residues), or any combination thereof. Thus, according to the present invention, a compound may be used to (a) reduce SUMOylation at one or more of residues K10, K116, K219, K337 and/or K394 of E4bp4, or a residue corresponding thereto, or any combination thereof; and/or reduce phosphorylation at one or more of residues S286, S301 and/or S454, or a residue corresponding thereto, or any combination thereof.

Any compound which is capable of altering or affecting the post-translational modification of E4bp4, wherein said alteration increases the activity of E4bp4 may be used according to the present invention. In some embodiments, said compound inhibits, reduces or ablates the phosphorylation and/or SUMOylation that occurs in wild-type (unmodified) E4bp4. Any appropriate kinase inhibitor may be used to inhibit, reduce or ablate phosphorylation of E4bp4. Suitable kinase inhibitors are known in the art and their selection would be routine to one of skill in the art. For example, based on current understanding of kinases which phosphorylate E4bp4, it may be appropriate to use inhibitors of phosphoinositide-dependent protein kinase-1 (PDK1) and/or casein kinase 1epsilon (CK1epsilon). Non-limiting examples of suitable kinase inhibitors include 4-(4-(2,3-Dihydrobenzo[1,4]dioxin-6-yl)-5-pyridin-2-yl-1H-imidazol-2-yl)benzamide (D4476) and 4,5,6,7-Tetrabromo-2-azabenzimidazole, 4,5,6,7-Tetrabromobenzotriazole (TBB).

### Increase in E4bp4 Activity

An increase in E4bp4 activity (e.g. as brought about by post-translational modification of E4bp4) may be measured relative to a control. Thus, the activity of E4bp4 in a sample of HPCs, an expanded NK cell population or in a sample obtained from an individual/patient to be treated according to the invention may be compared with the activity of E4bp4 in a control. Activity may be quantified in any appropriate terms, for example an increase in the expression of any downstream target of E4bp4. Any appropriate technique or method may be used for quantifying E4bp4 activity. Suitable techniques are known in the art, for example luciferase assays for quantifying expression of a reporter gene.

Typically the control is an equivalent population or sample which has not been treated according to the present invention. For example, in instances where a compound is used to alter or affect the post-translational modification of E4bp4, the corresponding control may be a population or sample in which no compound has been added to alter or affect the post-translational modification of E4bp4. As another example, in instances where a compound is used to inhibit the action of REV-ERB, the corresponding control may be a population or sample in which no compound has been added to inhibit the action of REV-ERB. As another example, in instances where a compound is used to inhibit the action of REV-ERB and a compound is used to alter or effect the post-translational modification of E4bp4, the corresponding control may be a population or sample in which no compound has been added to inhibit the action of REV-ERB or to alter or effect the post-translational modification of E4bp4.

The control may be as described herein. In the context of E4bp4 activity, the control may be an equivalent population or sample in which no increase in E4bp4 activity has been effected. As a non-limiting example, in the case where an individual/patient is treated with a compound that increases E4bp4 activity, a suitable control would be a different individual to which the compound has not been administered or the same individual prior to administration of the compound. Conventional methods for the ex vivo expansion of NK cells, including known methods may be considered control methods according to the present invention.

In the context of the present invention, a reference to increasing E4bp4 activity may be understood to mean that, the activity of E4bp4 is increased by at least 1.5-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, at least 4-fold, at least 5-fold, at least 6-fold, at least 7-fold, at least 8-fold, at least 9-fold, at least 10-fold or more relative to a control. Typically E4bp4 activity is increased by at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, or more compared with the control. E4bp4 activity may be measured indirectly by determining the increase in (CD16⁺) NK cell number. Thus, the number of (CD16⁺) NK cells may be increased by at least 1.5-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, at least 4-fold, at least 5-fold, at least 6-fold, at least 7-fold, at least 8-fold, at least 9-fold, at least 10-fold or more relative to a control. Typically the number of (CD16⁺) NK cells is increased by at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, or more compared with the control.

The activity of E4bp4 may be determined by quantitative and/or qualitative analysis, and may be measured directly or indirectly. The activity of E4bp4 relative to a control may be determined using any appropriate technique. Suitable standard techniques are known in the art.

The activity of E4bp4 may be increased compared with a control for at least 6 hours, at least 12 hours, at least 24 hours, at least 30 hours, at least 36 hours, at least 42 hours, at least 48 hours, at least 54 hours, at least 60 hours, at least 72 hours, at least 4 days, at least 5 days, at least 6 days, at least 1 week. Preferably, the activity of E4bp4 is increased for at least 12 to 72 hours. Typically this is assessed relative to the last administration of the compound which post-translationally modified E4bp4.

The activity of E4bp4 may be increased compared with a control for at least one, at least two, at least three, at least four, at least five, at least ten, at least 20, at least 30, at least 40 or more passages of the cultured cells. The activity of E4bp4 may be increased indefinitely.

### Methods of Expanding NK Cells

The invention relates to the production of NK cell populations comprising increased numbers of CD16⁺ NK cells. In particular, the present inventors have demonstrated that culturing HPCs for a defined period of time (a first culture period) in a medium which does not induce differentiation surprisingly results in the production of an expanded NK cell population with increased number of CD16⁺ NK cells.

Thus, the present invention provides a method for producing an expanded population of CD16⁺ NK cells, comprising the steps of: (a) culturing an HPC comprising sample obtained from an individual in medium which does not induce differentiation of the HPCs for a first culture period to produce a pre-differentiation HPC population; (b) culturing the pre-differentiation HPC population in medium which induces differentiation of the HPCs to NK for a second culture period. Step (b) typically comprises both the differentiation of the HPCs in the pre-differentiation HPC population and expansion of the resulting NK cells. Thus, the present invention provides a method for producing an expanded population of CD16⁺ NK cells, comprising the steps of: (a) culturing an HPC comprising sample obtained from an individual in medium which does not induce differentiation of the HPCs for a first culture period to produce a pre-differentiation HPC population; and (b) culturing the pre-differentiation HPC population in medium which induces differentiation of the HPCs to NK for a second culture period, wherein NK cell expansion also occurs in the second culture period. The present invention also provides a method for producing an expanded population of CD16⁺ NK cells, comprising the steps of: (a) culturing an HPC comprising sample obtained from an individual in medium which does not induce differentiation of the HPCs for a first culture period to produce a pre-differentiation HPC population; and (b) culturing the pre-differentiation HPC population in medium which induces differentiation of the HPCs to NK for a second culture period; and (c) expanding said cells in vitro to produce an expanded NK cell population.

The invention further provides a method for increasing the number of CD16⁺ NK cells in an expanded NK cell population, comprising the steps of: (a) culturing an HPC comprising sample obtained from an individual in medium which does not induce differentiation of the HPCs for a first culture period to produce a pre-differentiation HPC population; and (b) culturing the pre-differentiation HPC population in medium which induces differentiation of the HPCs to NK for a second culture period. Step (b) typically comprises both the differentiation of the HPCs in the pre-differentiation HPC population and expansion of the resulting NK cells. Thus, the present invention provides a method for increasing the number of CD16⁺ NK cells in an expanded NK cell population, comprising the steps of: (a) culturing an HPC comprising sample obtained from an individual in medium which does not induce differentiation of the HPCs for a first culture period to produce a pre-differentiation HPC population; (b) culturing the pre-differentiation HPC population in medium which induces differentiation of the HPCs to NK for a second culture period, wherein NK cell expansion also occurs in the second culture period. The present invention also provides a method for increasing the number of CD16⁺ NK cells in an expanded NK cell population, comprising the steps of: (a) culturing an HPC comprising sample obtained from an individual in medium which does not induce differentiation of the HPCs for a first culture period to produce a pre-differentiation HPC population; and (b) culturing the pre-differentiation HPC population in medium which induces differentiation of the HPCs to NK for a second culture period; and (c) expanding said cells in vitro to produce an expanded NK cell population.

The inventors have surprisingly demonstrated that culturing HPCs for a first culture period of between about 2 days to about 8 days in a medium which does not induce differentiation prior to differentiation of the pre-differentiation HPC population into NK cells increases the number of CD16⁺ NK cells in the resulting expanded NK cell population. As exemplified herein, the present inventors have found that prolonging this first culture period beyond 8 days decreases NK cell output and CD16 expression. Therefore, according to the present invention the duration of the first culture period (the pre-differentiation culture stage) is tightly controlled and precisely defined. According to the present invention, the first culture period (the pre-differentiation culture stage) typically does not exceed 8 days. In other words, step (a) of a method of the invention typically does not exceed 8 days.

Thus, the present invention provides an ex vivo method for producing an expanded population of CD16⁺ NK cells, comprising the steps of: (a) culturing an HPC comprising sample obtained from an individual in medium which does not induce differentiation of the HPCs for between about 2 days to about 8 days to produce a pre-differentiation HPC population; (b) culturing the pre-differentiation HPC population in medium which induces differentiation of the HPCs to NK for a second culture period, wherein NK cell expansion also occurs in the second culture period. The present invention also provides a method for producing an expanded population of CD16⁺ NK cells, comprising the steps of: (a) culturing an HPC comprising sample obtained from an individual in medium which does not induce differentiation of the HPCs for between about 2 days about 8 days to produce a pre-differentiation HPC population; and (b) culturing the pre-differentiation HPC population in medium which induces differentiation of the HPCs to NK for a second culture period; and (c) expanding said cells in vitro to produce an expanded NK cell population.

The invention further provides a method for increasing the number of CD16⁺ NK cells in an expanded NK cell population, comprising the steps of: (a) culturing an HPC comprising sample obtained from an individual in medium which does not induce differentiation of the HPCs for between about 2 days to about 8 days to produce a pre-differentiation HPC population; (b) culturing the pre-differentiation HPC population in medium which induces differentiation of the HPCs to NK for a second culture period, wherein NK cell expansion also occurs in the second culture period. The present invention also provides a method for increasing the number of CD16⁺ NK cells in an expanded NK cell population, comprising the steps of: (a) culturing an HPC comprising sample obtained from an individual in medium which does not induce differentiation of the HPCs for between about 2 to about 8 days to produce a pre-differentiation HPC population; and (b) culturing the pre-differentiation HPC population in medium which induces differentiation of the HPCs to NK for a second culture period; and (c) expanding said cells in vitro to produce an expanded NK cell population.

The first culture period is between about 2 days to about 8 days. Thus, the first culture period may be 2, 2.5, 3, 3.5, 4, 4.5, 5, 5.5, 6, 6.5, 7, 7.5 or 8 days or a period of any duration between 2 and 8 days. Preferably the first culture period may be between about 2 days to about 6 days (e.g. 2, 2.5, 3, 3.5, 4, 4.5, 5, 5.5 or 6 days or a period of any duration between 2 and 6 days, such as between about 4 to 6 days). More preferably the first culture period may be between about 2 days to about 4 days (e.g. 2, 2.5, 3, 3.5 or 4 days or a period of any duration between 2 and 4 days). The first culture period (the pre-differentiation culture stage, i.e. step (a)) typically does not exceed 8 days.

The first culture period is a period of time in which HPCs or an HPC comprising sample obtained from an individual is cultured in a medium which does not induce differentiation of the HPCs, typically wherein said medium does not induce differentiation of the HPCs to NK cells. Any culture medium suitable for the culture of HPCs may be used, provided that said medium does not induce the differentiation of the HPCs, typically to NK cells. Non-limiting examples of suitable media and factors for including in said media are described herein. This step of culturing HPCs or an HPC comprising sample obtained from an individual is cultured in a medium which does not induce differentiation of the HPCs, typically wherein said medium does not induce differentiation of the HPCs to NK cells is typically step (a) of a method of the invention.

As used herein, the term “pre-differentiation HPC population” is used to define a population of HPCs obtained by culturing HPCs or an HPC comprising sample obtained from an individual for a first culture period in a medium which does not induce differentiation of the HPCs, typically wherein said medium does not induce differentiation of the HPCs to NK cells. Said pre-differentiation HPC population may comprise other non-HPC cell types, provided that the HPCs are the predominant cell type (e.g. at least 50% of the cells are HPCs). Typically the pre-differentiation HPC population comprises less than 20%, less than 15%, less than 10%, less than 9%, less than 8%, less than 7%, less than 6%, less than 5%, less than 4%, less than 3%, less than 2%, less than 1%, less than 0.5% or fewer NK cells. Preferably the pre-differentiation HPC population comprises less than 2% NK cells, more preferably less than 1% NK cells. The numbers of HPCs and/or NK cells in the HPC comprising sample and/or the pre-differentiation HPC population can be determined by any appropriate technique, conventional examples include techniques such as FACS or flow cytometry.

Following the step of culturing an HPC comprising sample obtained from an individual in medium which does not induce differentiation of the HPCs for a first culture period (e.g. between about 2 days to about 8 days, such as between about 2 days to about 6 days, about 4 to about 6 days or about 2 days to about 4 days) to produce a pre-differentiation HPC population, an ex vivo method of the invention comprises a step of culturing the (pre-differentiation) HPCs or a pre-differentiation HPC population in a medium which induces differentiation of the (pre-differentiation) HPCs to NK cells for a second culture period.

The second culture period may be between about 10 days to about 30 days. Thus, the second culture period may be 10, 11, 12, 13, 14, 15, 16, 17, 18, 19, 20, 21, 22, 23, 24, 25, 26, 27, 28, 29 or 30 days or a period of any duration between 10 and 30 days, such as between 24 and 30 days. Preferably the second culture period may be between about 15 days to about 25 days (e.g. 15, 16, 17, 18, 19, 20, 21, 22, 23, 24 or 25 days or a period of any duration between 15 and 25 days). More preferably the second culture period may be between about 18 days to about 22 days (e.g. 18, 19, 20, 21 or 22 days or a period of any duration between 18 and 22 days). Most preferably, the second culture period is about 19 to 21 days, with about 20 days being particularly preferred.

Thus, the present invention provides a method for producing an expanded population of CD16⁺ NK cells, comprising the steps of: (a) culturing an HPC comprising sample obtained from an individual in medium which does not induce differentiation of the HPCs for a first culture period (e.g. between about 2 days to about 8 days, such as between about 2 days to about 6 days or about 2 days to about 4 days) to produce a pre-differentiation HPC population; and (b) culturing the pre-differentiation HPC population in medium which induces differentiation of the HPCs to NK for between about 15 days to about 30 days (e.g. between about 18 days to about 22 days, such as about 20 days). Step (b) typically comprises both the differentiation of the HPCs in the pre-differentiation HPC population and expansion of the resulting NK cells. Thus, the present invention provides a method for producing an expanded population of CD16⁺ NK cells, comprising the steps of: (a) culturing an HPC comprising sample obtained from an individual in medium which does not induce differentiation of the HPCs for a first culture period (e.g. between about 2 days to about 8 days, such as between about 2 days to about 6 days, between about 4 to 6 days, or about 2 days to about 4 days) to produce a pre-differentiation HPC population; (b) culturing the pre-differentiation HPC population in medium which induces differentiation of the HPCs to NK for between about 15 days to about 30 days (e.g. between about 18 days to about 22 days, such as about 20 days), wherein NK cell expansion also occurs in the second culture period. The present invention also provides a method for producing an expanded population of CD16⁺ NK cells, comprising the steps of: (a) culturing an HPC comprising sample obtained from an individual in medium which does not induce differentiation of the HPCs for a first culture period (e.g. between about 2 days to about 8 days, such as between about 2 days to about 6 days, between about 4 to 6 days, or about 2 days to about 4 days) to produce a pre-differentiation HPC population; and (b) culturing the pre-differentiation HPC population in medium which induces differentiation of the HPCs to NK for between about 15 days to about 30 days (e.g. between about 18 days to about 22 days, such as about 20 days); and (c) expanding said cells in vitro to produce an expanded NK cell population.

The invention further provides a method for increasing the number of CD16⁺ NK cells in an expanded NK cell population, comprising the steps of: (a) culturing an HPC comprising sample obtained from an individual in medium which does not induce differentiation of the HPCs for a first culture period (e.g. between about 2 days to about 8 days, such as between about 2 days to about 6 days or about 2 days to about 4 days) to produce a pre-differentiation HPC population; and (b) culturing the pre-differentiation HPC population in medium which induces differentiation of the HPCs to NK for between about 15 days to about 30 days (e.g. between about 18 days to about 22 days, such as about 20 days). Step (b) typically comprises both the differentiation of the HPCs in the pre-differentiation HPC population and expansion of the resulting NK cells. Thus, the present invention provides a method for increasing the number of CD16⁺ NK cells in an expanded NK cell population, comprising the steps of: (a) culturing an HPC comprising sample obtained from an individual in medium which does not induce differentiation of the HPCs for a first culture period (e.g. between about 2 days to about 8 days, such as between about 2 days to about 6 days, between about 4 to 6 days, or about 2 days to about 4 days) to produce a pre-differentiation HPC population; (b) culturing the pre-differentiation HPC population in medium which induces differentiation of the HPCs to NK for between about 15 days to about 30 days (e.g. between about 18 days to about 22 days, such as about 20 days), wherein NK cell expansion also occurs in the second culture period. The present invention also provides a method for increasing the number of CD16⁺ NK cells in an expanded NK cell population, comprising the steps of: (a) culturing an HPC comprising sample obtained from an individual in medium which does not induce differentiation of the HPCs for a first culture period (e.g. between about 2 days to about 8 days, such as between about 2 days to about 6 days, between about 4 to 6 days, or about 2 days to about 4 days) to produce a pre-differentiation HPC population; (b) culturing the pre-differentiation HPC population in medium which induces differentiation of the HPCs to NK for between about 15 days to about 30 days (e.g. between about 18 days to about 22 days, such as about 20 days); and (c) expanding said cells in vitro to produce an expanded NK cell population.

The second culture period is a period of time in which (pre-differentiation) HPCs or a pre-differentiation HPC population is cultured in a medium which induces differentiation of the (pre-differentiation) HPCs, typically wherein said medium induces differentiation of the (pre-differentiation) HPCs to NK cells. Any culture medium suitable for the culture of pre-differentiation HPCs may be used, provided that said medium induces the differentiation of the (pre-differentiation) HPCs, typically to NK cells. Non-limiting examples of suitable media and factors for including in said media are described herein. This step of culturing (pre-differentiation) HPCs or a pre-differentiation HPC population in a medium which induces differentiation of the (pre-differentiation) HPCs cells for a second culture period, typically wherein said medium induces differentiation of the (pre-differentiation) HPCs to NK cells is typically step (b) of a method of the invention.

A method of the invention typically also comprises expansion of the (CD16⁺) NK cells. By expansion, it is meant that the number of (CD16⁺) NK cells is increased. The increase in number of (CD16⁺) NK cells may be as described herein.

Expansion of the (CD16⁺) NK cells may occur in parallel (i.e. at the same time) as the differentiation of the (pre-differentiation) HPCs to NK cells. In other words, steps (b) and (c) may be simultaneous, such that the second culture period includes both differentiation and expansion, as described herein. Thus, the present invention provides a method for producing an expanded population of CD16⁺ NK cells, comprising the steps of: (a) culturing an HPC comprising sample obtained from an individual in medium which does not induce differentiation of the HPCs for a first culture period (e.g. between about 2 days to about 8 days, such as between about 2 days to about 6 days, between about 4 to 6 days, or about 2 days to about 4 days) to produce a pre-differentiation HPC population; and (b) culturing for a second culture period (e.g. between about 15 days to about 30 days, such as between about 18 days to about 22 days, such as about 20 days) the pre-differentiation HPC population in medium which induces differentiation of the HPCs to NK and expanding said cells in vitro to produce an expanded NK cell population. The invention also provides a method for increasing the number of CD16⁺ NK cells in an expanded NK cell population, comprising the steps of: (a) culturing an HPC comprising sample obtained from an individual in medium which does not induce differentiation of the HPCs for a first culture period (e.g. between about 2 days to about 8 days, such as between about 2 days to about 6 days, between about 4 to 6 days, or about 2 days to about 4 days) to produce a pre-differentiation HPC population; and (b) culturing for a second culture period (e.g. between about 15 days to about 30 days, such as between about 18 days to about 22 days, such as about 20 days) the pre-differentiation HPC population in medium which induces differentiation of the HPCs to NK and expanding said cells in vitro to produce an expanded NK cell population.

Alternatively, expansion of the (CD16⁺) NK cells may occur after the step of culturing the (pre-differentiation) HPCs or a pre-differentiation HPC population in a medium which induces differentiation of the (pre-differentiation) HPCs to NK cells for a second culture period. Thus, the present invention provides an ex vivo method for producing an expanded population of CD16⁺ NK cells, comprising the steps of: (a) culturing an HPC comprising sample obtained from an individual in medium which does not induce differentiation of the HPCs for a first culture period (e.g. between about 2 days to about 8 days, such as between about 2 days to about 6 days, between about 4 to 6 days, or about 2 days to about 4 days) to produce a pre-differentiation HPC population; (b) culturing the pre-differentiation HPC population in medium which induces differentiation of the HPCs to NK for a second culture period (e.g. between about 15 days to about 30 days, such as between about 18 days to about 22 days, such as about 20 days); and subsequently (c) expanding said cells in vitro to produce an expanded NK cell population. The invention further provides an ex vivo method for increasing the number of CD16⁺ NK cells in an expanded NK cell population, comprising the steps of: (a) culturing an HPC comprising sample obtained from an individual in medium which does not induce differentiation of the HPCs for a first culture period (e.g. between about 2 days to about 8 days, such as between about 2 days to about 6 days, between about 4 to 6 days, or about 2 days to about 4 days) to produce a pre-differentiation HPC population; (b) culturing the pre-differentiation HPC population in medium which induces differentiation of the HPCs to NK for a second culture period (e.g. between about 15 days to about 30 days, such as between about 18 days to about 22 days, such as about 20 days); and subsequently (c) expanding said cells in vitro to produce an expanded NK cell population.

The expansion of the NK cells may comprise culturing the (CD16⁺) NK cells for a period may of between about 10 days to about 30 days. As discussed herein, this may be in parallel (simultaneous with) the differentiation step, or subsequent to the differentiation step. In either case, the expansion period may be 10, 11, 12, 13, 14, 15, 16, 17, 18, 19, 20, 21, 22, 23, 24, 25, 26, 27, 28, 29 or 30 days or a period of any duration between 10 and 30 days, such as between 24 and 30 days. Preferably the expansion period may be between about 15 days to about 25 days (e.g. 15, 16, 17, 18, 19, 20, 21, 22, 23, 24 or 25 days or a period of any duration between 15 and 25 days). More preferably the expansion period may be between about 18 days to about 22 days (e.g. 18, 19, 20, 21 or 22 days or a period of any duration between 18 and 22 days). Most preferably, the expansion period is about 19 to 21 days, with about 20 days being particularly preferred.

The expansion period is a period of time in which (CD16⁺) NK cells are cultured in a medium which induces their expansion. Any culture medium suitable for the culture of (CD16⁺) NK cells may be used, provided that said medium induces the expansion of (CD16⁺) NK cells. Non-limiting examples of suitable media and factors for including in said media are described herein. The expansion medium may be the same as the medium used for the second culture period, i.e. the same medium which induces differentiation of the (pre-differentiation) HPCs to NK cells. Typically expansion involves culturing the cells in cytokines and growth factors associated with NK cell development, such as IL-15, and may involve transferring the pre-differentiation HPCs or the NK cells to a suitable stromal (support) cell layer, such as OP9 or EL08 stromal cells, preferably EL08 cells (e.g. EL08-ID2 cells), or a cell-free alternative support layer such as an ECM as described herein. The expansion step typically lasts for the remainder of the ex vivo culture period (as defined herein). The culture medium may be changed as often as required during this stage in order to facilitate NK cell expansion. Any REV-ERB inhibitory compounds, Notch ligands and/or compounds which alter the posttranslational modification of E4bp4 that are present in the culture medium before it is replaced may be administered again with the fresh culture medium, either at the same or different concentration. Preferably, the REV-ERB inhibitory compounds, Notch ligands and/or compounds which alter the posttranslational modification of E4bp4 used in step (a) and/or (b) are not present during the expansion phase.

The present invention relates to methods for expanding a (CD16⁺) NK cell population and/or for increasing the number of (CD16⁺) NK cells in an expanded NK cell population. Said method may be in vitro, in vivo or ex vivo. Typically the method of the invention is ex vivo.

Steps (a) and (b) (where expansion is comprised in step (b)) or steps (a) to (c) (when expansion is subsequent to step (b)) of the methods of the invention are typically carried out in order. In other words, the HPC comprising sample is typically first cultured in medium which does not induce differentiation of the HPCs (to NK cells) for a first culture period to produce a pre-differentiation HPC population, followed by culturing of the pre-differentiation HPC population in medium which induces differentiation of the HPCs to NK cells for a second culture period, which may be followed by a separate step of expanding the NK cells in vitro. As described herein, preferably the NK cell expansion step is simultaneous or concurrent with the step of culturing of the pre-differentiation HPC population in medium which induces differentiation of the HPCs to NK cells for a second culture period, i.e. step (b) and (c) are simultaneous/concurrent, or at least partially overlap.

The durations of step (a) and step (b) (where expansion is comprised in step (b)) or step (a), step (b) and step (c) (when expansion is subsequent to step (b)) are independent, and any duration of step (a) above may be used in combination with any duration of step (b) above and/or any duration of step (c) above.

An ex vivo method of the invention may be at least 12 days in length (including steps (a), (b) and an expansion step when this is not concurrent with step (b)). By way of non-limiting example, an ex vivo method of the invention may be at least 14, at least 16, at least 18, at least 22, at least 24, at least 26, at least 28, at least 30, at least 32, at least 34, at least 36, or more days in length. By way of further non-limiting example, an ex vivo method of the invention may be between about 12 to about 36 days in length, such as between about 12 to about 28 days, between about 12 to about 26 days, between about 12 to about 24 days, between about 12 to about 22 days, between about 12 to about 18 days, between about 12 to about 16 days, or between about 12 to about 14 days.

In all methods of the invention, the sample comprising HPCs obtained from an individual/patient may be a sample obtained from bone marrow, cord blood and/or peripheral blood. Thus, the sample may be a cord or peripheral blood sample, or a bone marrow sample or biopsy. The sample may be obtained from the individual who is to be treated with the NK cell population produced by a method of the invention (i.e. a patient). Alternatively, the sample is obtained from a healthy individual.

The sample comprising HPCs may be treated prior to its use in the present methods. By way of non-limiting example, the sample comprising the HPCs may be frozen once it has been obtained from the subject. Samples which are frozen will typically require thawing prior to use in a method of the invention. Following thawing a sample may require plating and/or an initial culture period to allow the HPCs to recover from freezing prior to commencing a method of the invention. This initial culture period may be referred to as a “recovery period”. Methods of the invention may include such a recovery period, or may not include such a recovery period. The recovery period, when present, may be one week, up to six days, up to five days, up to four days, up to three days, up to two days, up to one day in duration. A recovery period may preferably be up to 2 days, i.e. up to 1 day, up to 12 hours, up to 8 hours in duration. If a recovery period is not comprised in a method of the invention, step (a) of said method may begin on the same day as the date the method commences.

Step (a) of a method of the invention may begin on the same day as the method commences (e.g. from thawing and plating the HPCs or isolating the HPCs in the sample). Step (a) of a method of the invention may begin within one week, within six days, within five days, within four days, within three days, within two days, within one day, or on the same day as the date the method commences (e.g. from the date of thawing and plating the HPCs, the date of isolating the HPCs in the sample, or on the same day as isolating the NK cell precursors). The date of isolating the HPCs and/or NK cell precursors is typically the same day that the sample is obtained from the patient.

Step (a) of a method of the invention, typically begins following a recovery period as defined herein. Thus, step (a) of a method of the invention may begin within one week, within six days, within five days, within four days, within three days, within two days, within one day, within 12 hours or within 8 hours of commencement of the method (e.g. from thawing and plating the HPCs, the date of isolating the HPCs in the sample, or isolating the NK cell precursors). Preferably, step (a) of a method of the invention may begin within two days, within one day, within 12 hours or within 8 hours of commencement of the method, more preferably about 1 day following commencement of the method.

According to the present invention, a sample comprising HPCs is any sample from an individual which comprises a sufficient number of HPCs (as described herein), such that an expanded (CD16⁺) NK cell population can be obtained by a method of the present invention. Typically the sample comprises HSCs. Preferably said sample is enriched for HSCs, such as a cord or peripheral blood sample or a bone marrow sample or biopsy as described herein.

A method of the invention may result in an increase in the number of CD16⁺ NK cells of at least 1.5-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, at least 4-fold, at least 5-fold, at least 6-fold, at least 7-fold, at least 8-fold, at least 9-fold, at least 10-fold or more relative to a control. Typically the number of NK cells is increased by at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, or more compared with the control.

A method of the invention may result in an increase in the number of CD16⁺ NK cells in an expanded NK cell population compared with the number of CD16⁺ NK cells in a control expanded NK cell population. A method of the invention may result in an increase in the proportion of CD16⁺ NK cells in an expanded NK cell population compared with the number of CD16⁺ NK cells in a control expanded NK cell population. A control expanded NK cell population may be as described herein. A control expanded NK cell population may be an expanded NK cell population produced by a control (e.g.) conventional method. Typically, a control expanded NK cell population may be an expanded NK cell population produced by a corresponding method to those described herein, but wherein (i) the step of culturing the HPC comprising sample in medium which does not induce differentiation of the HPCs for a first culture period (i.e. step (a)) is omitted; or (ii) the step of culturing the HPC comprising sample in medium which does not induce differentiation of the HPCs is carried out for less than about 2 days or for more than about 8 days.

A method of the invention may result in an increase in the number or proportion of CD16⁺ NK cells in an expanded NK cell population by at least 1.5-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, at least 4-fold, at least 5-fold, at least 6-fold, at least 7-fold, at least 8-fold, at least 9-fold, at least 10-fold or more relative to a control expanded NK cell population, or an expanded NK cell population produced by a control method. Typically the number of NK cells is increased by at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, or more at least 2×, 2.5×, 3×, 4×, 5×, 6×, 7×, 8×, 9×, 10× or more compared with a control expanded NK cell population, or an expanded NK cell population produced by a control method.

A method of the invention produces an expanded (CD16⁺) NK cell population as described herein. Typically, a method of the invention produces an expanded (CD16⁺) NK cell population wherein at least 10% of the NK cells in the population are CD16⁺ NK cells, at least 15% of the NK cells in the population are CD16⁺ NK cells, at least 20% of the NK cells in the population are CD16⁺ NK cells, at least 25% of the NK cells in the population are CD16⁺ NK cells, at least 30% of the NK cells in the population are CD16⁺ NK cells, at least 35% of the NK cells in the population are CD16⁺ NK cells, at least 40% of the NK cells in the population are CD16⁺ NK cells, at least 45% of the NK cells in the population are CD16⁺ NK cells, at least 50% of the NK cells in the population are CD16⁺ NK cells, at least 60% of the NK cells in the population are CD16⁺ NK cells, at least 70% of the NK cells in the population are CD16⁺ NK cells, at least 80% of the NK cells in the population are CD16⁺ NK cells, up to 100% of the NK cells in the population are CD16⁺ NK cells. Preferably at least 15% of the NK cells are CD16⁺ NK cells, more preferably at least 20% of the NK cells are CD16⁺ NK cells, still more preferably at least 25% of the NK cells are CD16⁺ NK cells even more preferably at least 30% of the NK cells are CD16⁺ NK cells.

The methods of the invention produce increased numbers of CD16⁺ NK cells without the need for introduction of exogenous nucleic acid (such as by transduction or transfection). This is in contrast to conventional methods for expanding NK cells with increased CD16 expression, which require expression of an exogenous CD16 transgene to achieve even minimal increases in CD16 expression.

Accordingly, the invention provides method for the production of expanded CD16⁺ NK cells, and methods for increasing the number of CD16⁺ NK cells in an expanded NK cell population, wherein said method does not comprise a step of contacting with and/or introducing to the HPCs and/or NK cells an exogenous nucleic acid (typically a nucleic acid encoding for CD16).

A method of the invention may accelerate the production of phenotypically mature NK cells. In other words, the method of the invention may reduce the time taken to arrive at a population of mature NK cells. A reduction in the run time of the method offers a further advantage over the conventional methods for NK cell expansion known in the art.

An ex vivo method of the present invention may comprise one or more additional steps. By way of non-limiting example, an ex vivo method of the invention may comprise one or more further, initial, step(s) of isolating and/or enriching HPCs from a sample (e.g. a peripheral or cord blood sample). By way of further non-limiting example, an ex vivo method of the present invention may comprise a further, typically final, step to purify the expanded (CD16⁺) NK cell population. This ensures a pure population for therapeutic administration as described herein. Purification of the expanded NK cell population may be by any appropriate means. Standard cell purification methods are known in the art, such as cell sorting, including fluorescence-activated cell sorting (FACS) and magnetic-activated cell sorting (MACS).

In some methods of the invention no REV-ERB inhibitory compound is used in step (a) and/or in step (b). In some methods of the invention, no REV-ERB inhibitory compound is used in either step (a) or step (b). Any and all disclosure here in relation to methods of the invention applies equally and without reservation to methods in which no REV-ERB inhibitory compound is used, unless stated to the contrary.

In some methods of the invention no Notch ligand (e.g. DLL4) is used in step (a) and/or in step (b). In some methods of the invention, no Notch ligand (e.g. DLL4) is used in either step (a) or step (b). Any and all disclosure here in relation to methods of the invention applies equally and without reservation to methods in which no Notch ligand (e.g. DLL4) is used, unless stated to the contrary.

In some methods of the invention no REV-ERB inhibitory compound and no Notch ligand (e.g. DLL4) is used in step (a) and/or in step (b). In some methods of the invention, no REV-ERB inhibitory compound and no Notch ligand (e.g. DLL4) is used in either step (a) or step (b). Any and all disclosure here in relation to methods of the invention applies equally and without reservation to methods in which no REV-ERB inhibitory compound and no Notch ligand (e.g. DLL4) is used, unless stated to the contrary. In embodiments where no REV-ERB inhibitory compound and no Notch ligand (e.g. DLL4) is used, the inclusion of a step of culturing HPCs in medium which does not include differentiation of the HPCs is sufficient to increase the number of CD16⁺ NK cells, i.e. the inclusion of step (a) is sufficient to increase the number of CD16⁺ NK cells in an expanded NK cell population of the invention.

In some methods of the invention, including but not limited to those involving the combination of a Notch ligand and a REV-ERB inhibitory compound, the % of NK cells in the final cell population may be very high (typically greater than 85%, preferably greater than 90%, more preferably greater than 95%, and may approach 100%). In such instances, a final purification step may optionally be omitted.

### Methods Using Notch Ligand

The methods for producing an expanded population of CD16⁺ NK cells and/or the method for increasing the number of CD16⁺ NK cells in an expanded NK cell population may involve culturing NK cell precursors (HPCs) in the presence of a Notch ligand as described herein (e.g. DLL4). Wherein a Notch ligand (e.g. DLL4) is used, a method may comprise contacting HPCs with the Notch ligand (e.g. DLL4). These methods of the invention allow for the rapid expansion of NK cells, reducing the time needed for their culture, and hence the risk of exhaustion, enhancing the cytotoxicity of the NK cells when transfused into a patient.

The HPCs may be cultured in the presence of the Notch ligand (e.g. DLL4) for at least part of a first culture period in a method described herein. Thus, the Notch ligand (e.g. DLL4) may be comprised in the medium which does not induce differentiation of the HPCs.

Thus, the present invention provides a method for producing an expanded population of CD16⁺ NK cells, comprising the steps of: (a) culturing an HPC comprising sample obtained from an individual in medium which does not induce differentiation of the HPCs for a first culture period to produce a pre-differentiation HPC population; and (b) culturing the pre-differentiation HPC population in medium which induces differentiation of the HPCs to NK for a second culture period. Step (b) typically comprises both the differentiation of the HPCs in the pre-differentiation HPC population and expansion of the resulting NK cells. Thus, the present invention provides a method for producing an expanded population of CD16⁺ NK cells, comprising the steps of: (a) culturing an HPC comprising sample obtained from an individual in medium which does not induce differentiation of the HPCs for a first culture period to produce a pre-differentiation HPC population; (b) culturing the pre-differentiation HPC population in medium which induces differentiation of the HPCs to NK for a second culture period, wherein NK cell expansion also occurs in the second culture period. The present invention also provides a method for producing an expanded CD16⁺ NK cell population, comprising the steps of: (a) culturing an HPC comprising sample obtained from an individual in medium which does not induce differentiation of the HPCs for a first culture period to produce a pre-differentiation HPC population; and (b) culturing the pre-differentiation HPC population in medium which induces differentiation of the HPCs to NK for a second culture period; and (c) expanding said cells in vitro to produce an expanded NK cell population. For at least part of the first culture period, the HPCs may also be contacted with (cultured in the presence of) a Notch ligand (e.g. DLL4). Thus the medium which does not induce differentiation of the HPCs may comprise a Notch ligand (e.g. DLL4). Thus, the HPCs may be contacted with (cultured in the presence of) a Notch ligand (e.g. DLL4) for at least part of step (a) of a method of the invention.

The invention also provides a method for increasing the number of CD16⁺ NK cells in an expanded NK cell population, comprising the steps of: (a) culturing an HPC comprising sample obtained from an individual in medium which does not induce differentiation of the HPCs for a first culture period to produce a pre-differentiation HPC population; and (b) culturing the pre-differentiation HPC population in medium which induces differentiation of the HPCs to NK for a second culture period. Step (b) typically comprises both the differentiation of the HPCs in the pre-differentiation HPC population and expansion of the resulting NK cells. Thus, the present invention provides a method for increasing the number of CD16⁺ NK cells in an expanded NK cell population, comprising the steps of: (a) culturing an HPC comprising sample obtained from an individual in medium which does not induce differentiation of the HPCs for a first culture period to produce a pre-differentiation HPC population; (b) culturing the pre-differentiation HPC population in medium which induces differentiation of the HPCs to NK for a second culture period, wherein NK cell expansion also occurs in the second culture period. The present invention also provides a method for increasing the number of CD16⁺ NK cells in an expanded NK cell population, comprising the steps of: (a) culturing an HPC comprising sample obtained from an individual in medium which does not induce differentiation of the HPCs for a first culture period to produce a pre-differentiation HPC population; and (b) culturing the pre-differentiation HPC population in medium which induces differentiation of the HPCs to NK for a second culture period; and (c) expanding said cells in vitro to produce an expanded NK cell population. For at least part of the first culture period, the HPCs may also be contacted with (cultured in the presence of) a Notch ligand (e.g. DLL4). Thus the medium which does not induce differentiation of the HPCs may comprise a Notch ligand (e.g. DLL4). Thus, the HPCs may be contacted with (cultured in the presence of) a Notch ligand (e.g. DLL4) for at least part of step (a) of a method of the invention.

As described herein, the first culture period is between about 2 days to about 8 days. The HPCs may be contacted with a Notch ligand (e.g. DLL4) for at least part of the first culture period, up to the entirety of the first culture period. Thus, the HPCs may be contacted with a Notch ligand (e.g. DLL4) for 2, 2.5, 3, 3.5, 4, 4.5, 5, 5.5, 6, 6.5, 7, 7.5 or 8 days of the first culture period may or a period of any duration between 2 and 8 days. Preferably the HPCs may be contacted with a Notch ligand (e.g. DLL4) for between about 2 days to about 6 days (e.g. 2, 2.5, 3, 3.5, 4, 4.5, 5, 5.5 or 6 days or a period of any duration between 2 and 6 days, such as between about 4 to 6 days) of the first culture period. More preferably the HPCs may be contacted with a Notch ligand (e.g. DLL4) for between about 2 days to about 4 days (e.g. 2, 2.5, 3, 3.5 or 4 days or a period of any duration between 2 and 4 days) of the first culture period.

Most preferably, the first culture period comprises a period of between about 0 to about 2 days, particularly about 1 day, in which a Notch ligand (e.g. DLL4) is absent, followed by contacting the HPCs with the Notch ligand (e.g. DLL4) for the remainder of the first culture period. Thus, a Notch ligand may be added to the medium which does not induce differentiation of the HPCs after a period of between about 0 to about 2 days, particularly the Notch ligand may be added to the medium which does not induce differentiation of the HPCs after about 1 day.

A method of the invention may comprise a recovery period, such that the HPCs are culture for a recovery period of between about 0 days to about 2 days before being contacted with a Notch ligand (e.g. DLL4) and cultured for a first culture period. As described herein, the first culture period is between about 2 days to about 8 days. The HPCs may be contacted with a Notch ligand (e.g. DLL4) for at least part of the first culture period, up to the entirety of the first culture period. Thus, the HPCs may be contacted with a Notch ligand (e.g. DLL4) for 2, 2.5, 3, 3.5, 4, 4.5, 5, 5.5, 6, 6.5, 7, 7.5 or 8 days of the first culture period may or a period of any duration between 2 and 8 days. Preferably the HPCs may be contacted with a Notch ligand (e.g. DLL4) for between about 2 days to about 6 days (e.g. 2, 2.5, 3, 3.5, 4, 4.5, 5, 5.5 or 6 days or a period of any duration between 2 and 6 days, such as, between about 4 to 6 days) of the first culture period. More preferably the HPCs may be contacted with a Notch ligand (e.g. DLL4) for between about 2 days to about 4 days (e.g. 2, 2.5, 3, 3.5 or 4 days or a period of any duration between 2 and 4 days) of the first culture period. A method may comprise a recovery period of between about 0 to about 2 days, particularly about 1 day, in which a Notch ligand (e.g. DLL4) is absent, followed by contacting the HPCs with the Notch ligand (e.g. DLL4) for a first culture period. Thus, a Notch ligand may be added to the medium which does not induce differentiation of the HPCs after a period of between about 0 to about 2 days, particularly the Notch ligand may be added to the medium which does not induce differentiation of the HPCs after about 1 day.

If the medium which does not induce differentiation of the HPCs is replaced during the first culture period, then the replacement medium which does not induce differentiation of the HPCs may comprise/not comprise Notch ligand (e.g. DLL4), depending on whether the presence of the Notch ligand (e.g. DLL4) is desired at that point within the first culture period. By way of non-limiting example, if the medium which does not induce differentiation of the HPCs is replaced on day 2, but the HPCs are to be cultured with the Notch ligand (e.g. DLL4) only from day 4, then the replacement medium which does not induce differentiation of the HPCs at day 2 would not include the Notch ligand (e.g. DLL4). By way of further limiting example, alternatively or in addition, if the medium which does not induce differentiation of the HPCs is replaced on day 6, and the HPCs are to be cultured with the Notch ligand (e.g. DLL4) from day 4, then the replacement medium which does not induce differentiation of the HPCs at day 6 would include the Notch ligand (e.g. DLL4). The Notch ligand (e.g. DLL4) may be added to the medium which does not induce differentiation of the HPCs before it is used to replace that on the HPCs, or the medium which does not induce differentiation of the HPCs may be replaced, and then Notch ligand (e.g. DLL4) then added to the HPCs.

Alternatively or in addition, the pre-differentiation HPC population may be cultured in the presence of the Notch ligand (e.g. DLL4) for at least part of a second culture period in a method described herein. Thus, the Notch ligand (e.g. DLL4) may be comprised in the medium which does induce differentiation of the HPCs.

Accordingly, the present invention provides a method for producing an expanded population of CD16⁺ NK cells, comprising the steps of: (a) culturing an HPC comprising sample obtained from an individual in medium which does not induce differentiation of the HPCs for a first culture period to produce a pre-differentiation HPC population; and (b) culturing the pre-differentiation HPC population in medium which induces differentiation of the HPCs to NK for a second culture period. Step (b) typically comprises both the differentiation of the HPCs in the pre-differentiation HPC population and expansion of the resulting NK cells. Thus, the present invention provides a method for producing an expanded population of CD16⁺ NK cells, comprising the steps of: (a) culturing an HPC comprising sample obtained from an individual in medium which does not induce differentiation of the HPCs for a first culture period to produce a pre-differentiation HPC population; (b) culturing the pre-differentiation HPC population in medium which induces differentiation of the HPCs to NK for a second culture period, wherein NK cell expansion also occurs in the second culture period. The present invention also provides a method for providing an expanded CD16⁺ NK cell population, comprising the steps of: (a) culturing an HPC comprising sample obtained from an individual in medium which does not induce differentiation of the HPCs for a first culture period to produce a pre-differentiation HPC population; and (b) culturing the pre-differentiation HPC population in medium which induces differentiation of the HPCs to NK for a second culture period; and (c) expanding said cells in vitro to produce an expanded NK cell population. For at least part of the second culture period, the pre-differentiation HPCs may also be contacted with (cultured in the presence of) a Notch ligand (e.g. DLL4). Thus the medium which does induce differentiation of the HPCs may comprise a Notch ligand (e.g. DLL4). Thus, the pre-differentiation HPCs may be contacted with (cultured in the presence of) a Notch ligand (e.g. DLL4) for at least part of step (b) of a method of the invention. This may be as an alternative or in addition to the inclusion of a Notch ligand (e.g. DLL4) in step (a) as described herein.

The invention also provides a method for increasing the number of CD16⁺ NK cells in an expanded NK cell population, comprising the steps of: (a) culturing an HPC comprising sample obtained from an individual in medium which does not induce differentiation of the HPCs for a first culture period to produce a pre-differentiation HPC population; and (b) culturing the pre-differentiation HPC population in medium which induces differentiation of the HPCs to NK for a second culture period. Step (b) typically comprises both the differentiation of the HPCs in the pre-differentiation HPC population and expansion of the resulting NK cells. Thus, the present invention provides a method for increasing the number of CD16⁺ NK cells in an expanded NK cell population, comprising the steps of: (a) culturing an HPC comprising sample obtained from an individual in medium which does not induce differentiation of the HPCs for a first culture period to produce a pre-differentiation HPC population; (b) culturing the pre-differentiation HPC population in medium which induces differentiation of the HPCs to NK for a second culture period, wherein NK cell expansion also occurs in the second culture period. The present invention also provides a method for increasing the number of CD16⁺ NK cells in an expanded NK cell population, comprising the steps of: (a) culturing an HPC comprising sample obtained from an individual in medium which does not induce differentiation of the HPCs for a first culture period to produce a pre-differentiation HPC population; and (b) culturing the pre-differentiation HPC population in medium which induces differentiation of the HPCs to NK for a second culture period; and (c) expanding said cells in vitro to produce an expanded NK cell population. For at least part of the second culture period, the pre-differentiation HPCs may also be contacted with (cultured in the presence of a Notch ligand (e.g. DLL4). Thus the medium which does induce differentiation of the HPCs may comprise a Notch ligand (e.g. DLL4). Thus, the pre-differentiation HPCs may be contacted with (cultured in the presence of) a Notch ligand (e.g. DLL4) for at least part of step (b) of a method of the invention. This may be as an alternative or in addition to the inclusion of a Notch ligand (e.g. DLL4) in step (a) as described herein.

As described herein, the second culture period may be between about 10 days to about 30 days. The pre-differentiation HPCs may be contacted with a Notch ligand (e.g. DLL4) for at least part of the second culture period, up to the entirety of the second culture period. Thus, the pre-differentiation HPCs may be contacted with a Notch ligand (e.g. DLL4) for 10, 11, 12, 13, 14, 15, 16, 17, 18, 19, 20, 21, 22, 23, 24, 25, 26, 27, 28, 29 or 30 days of the second culture period, or a period of any duration between 10 and 30 days. Preferably the pre-differentiation HPCs may be contacted with a Notch ligand (e.g. DLL4) for between about 15 days to about 25 days (e.g. 15, 16, 17, 18, 19, 20, 21, 22, 23, 24 or 25 days or a period of any duration between 15 and 25 days) or between about 24 to about 30 days (e.g. 24, 25, 26, 27, 28, 29 or 30 days, or a period of any duration between 24 and 30 days) of the second culture period. More preferably the pre-differentiation HPCs may be contacted with a Notch ligand (e.g. DLL4) for between about 18 days to about 22 days (e.g. 18, 19, 20, 21 or 22 days or a period of any duration between 18 and 22 days) of the second culture period. Most preferably, the pre-differentiation HPCs may be contacted with a Notch ligand (e.g. DLL4) for between about 19 to 21 days of the second culture period, with about 20 days being particularly preferred.

Preferably a Notch ligand (e.g. DLL4) is present only before differentiation of the HPCs is induced. In other words, a Notch ligand (e.g. DLL4) is present only for at least part of the first culture period, and preferably not for at least part of the second culture period. Thus, a Notch ligand (e.g. DLL4) is preferably present in the medium which does not induce differentiation of the HPCs, and not the medium which does induce differentiation of the HPCs. The pre-differentiation HPC population resulting from step (a) of a method of the invention may be transferred to a different culture vessel prior to step (b). This reduces the risk of contamination of the Notch ligand in step (b) of the method.

Typically the Notch ligand is a Notch ligand as described herein. Preferably, the Notch ligand is DDL4, or a fragment thereof which retains the function of DLL4, as described herein.

The Notch ligand (such as DLL4) may be present in solution (e.g. in the culture medium) or used to coat the vessel in which the HPCs are cultured. Preferably the Notch ligand (e.g. DLL4) is used to coat the vessel in which the HPCs are cultured. Any appropriate concentration of Notch ligand may be used. As a non-limiting example, in any aspect of the invention where a Notch ligand is used, the Notch ligand (e.g. DLL4) may be used at a concentration of about 1 μg/ml to about 100 μg/ml, about 1 μg/ml to about 50 μg/ml, about 1 μg/ml to about 25 μg/ml, about 1 μg/ml to about 10 μg/ml or less. In some embodiments the Notch ligand (e.g. DLL4) is used at a concentration of about 50 μg/ml, about 25 μg/ml, about 20 μg/ml, about 15 μg/ml, about 10 μg/ml, about 5 μg/ml, about 2 μg/ml, preferably about 10 μg/ml, more preferably about 2 μg/ml. The Notch ligand of the invention (e.g. DLL4) may be coated directly onto tissue culture plastic. Alternatively, additional substrates and/or linkers may be used to facilitate the attachment of the Notch ligand (such as DLL4) to the surface of the culture vessels. Examples of such substrates are known in the art, such as poly-L-lysine.

As described above, HPCs may be cultured in the presence or absence of a stromal support cell or feeder cell, or population thereof. In some preferred embodiments where a Notch ligand is used, the cells are cultured in the absence of a stromal support cell or population thereof, but optionally may be cultured with an ECM as described herein.

The Notch ligand of the invention may be added to the sample comprising HPCs within one week, within six days, within five days, within four days, within three days, within two days, within one day of commencing a method of the invention. For example, from the date of thawing and plating the HPCs, the date of isolating the HPCs in the sample, or on the same day as isolating the NK cell precursors. The date of isolating the HPCs and/or NK cell precursors is typically the same day that the sample is obtained from the patient. Preferably the Notch ligand of the invention is added to the sample within four days of commencing a method of the invention (e.g. from thawing and plating the HPCs or isolating the HPCs in the sample), such as on the day of commencement (day 0), or day one or two following commencement (e.g. from thawing and plating the HPCs or isolation of the HPCs). Most preferably the Notch ligand of the invention is added to the sample one day post commencement (e.g. from thawing and plating the HPCs or isolation of the HPCs). Thus, typically the Notch ligand is present on or from one day post commencement (e.g. from thawing and plating the HPCs or isolation of the HPCs), until the pre-differentiation HPC population is cultured in the medium which induces differentiation of the pre-differentiation HPCs to NK cells (i.e. from day one post commencement (e.g. from thawing and plating the HPCs or isolation) until the end of step (a)/start of step (b)).

The cells (e.g. the HPCs in step (a)) may be cultured in the presence of a Notch ligand (such as DLL4) for at least 6 hours, at least 12 hours, at least 24 hours, at least 30 hours, at least 36 hours, at least 42 hours, at least 48 hours, at least 54 hours, at least 60 hours, at least 72 hours, at least 4 days, at least 5 days, at least 6 days, at least 7 days, or 8 days. Typically for between 1-7 days, preferably for about 4 days.

Alternatively, these durations may be measured in terms of the number of cell passages. For example, at least one, at least two, at least three, at least four, at least five, at least ten, at least 20, or more passages of the cells (either in vivo, or cultured ex vivo or in vitro). Typically, these durations may be up to 10 passages of the cells, i.e. any number of passages between 1 and 10 (1, 2, 3, 4, 5, 6, 7, 8, 9 or 10 passages), preferably 1, 2 or 3 passages.

The present invention provides a method for producing an expanded population of CD16⁺ NK cells, comprising the steps of: (a) culturing an HPC comprising sample obtained from an individual in medium which does not induce differentiation of the HPCs for a first culture period to produce a pre-differentiation HPC population, wherein (i) a Notch ligand (e.g. DLL4) is added on day 1 post-commencement or wherein the HPCs are transferred to a culture vessel coated with said Notch ligand (e.g. DLL4) on day 1 post-commencement and cultured in contact with the Notch ligand (e.g. DLL4) for 4 days; and (b) culturing the pre-differentiation HPC population in medium which induces differentiation of the HPCs to NK for a second culture period to allow both the differentiation of the HPCs in the pre-differentiation HPC population and expansion of the resulting NK cells. Wherein (i) the medium which does not induce differentiation of the HPCs does not contain a REV-ERB inhibitory compound; and (ii) the medium which induces differentiation of the HPCs to NK does not comprise either a REV-ERB inhibitory compound or a Notch ligand.

The invention also provides a method for increasing the number of CD16⁺ NK cells in an expanded NK cell population, comprising the steps of: (a) culturing an HPC comprising sample obtained from an individual in medium which does not induce differentiation of the HPCs for a first culture period to produce a pre-differentiation HPC population, wherein (i) a Notch ligand (e.g. DLL4) is added on day 1 post-commencement or wherein the HPCs are transferred to a culture vessel coated with said Notch ligand (e.g. DLL4) on day 1 post-commencement and cultured in contact with the Notch ligand (e.g. DLL4) for 4 days; and (b) culturing the pre-differentiation HPC population in medium which induces differentiation of the HPCs to NK for a second culture period to allow both the differentiation of the HPCs in the pre-differentiation HPC population and expansion of the resulting NK cells. Wherein (i) the medium which does not induce differentiation of the HPCs does not contain a REV-ERB inhibitory compound; and (ii) the medium which induces differentiation of the HPCs to NK does not comprise either a REV-ERB inhibitory compound or a Notch ligand.

Methods Using Compounds which Inhibit REV-ERB Activity

As described herein, the methods of the invention may not comprise the use of a compound which inhibits REV-ERB activity. However, in some embodiments, the HPCs may be cultured in the presence of a compound which inhibits REV-ERB activity for at least part of a first culture period in a method described herein. Thus, the compound which inhibits REV-ERB activity may be comprised in the medium which does not induce differentiation of the HPCs.

Thus, the present invention provides a method for producing an expanded population of CD16⁺ NK cells, comprising the steps of: (a) culturing an HPC comprising sample obtained from an individual in medium which does not induce differentiation of the HPCs for a first culture period to produce a pre-differentiation HPC population; and (b) culturing the pre-differentiation HPC population in medium which induces differentiation of the HPCs to NK for a second culture period. Step (b) typically comprises both the differentiation of the HPCs in the pre-differentiation HPC population and expansion of the resulting NK cells. Thus, the present invention provides a method for producing an expanded population of CD16⁺ NK cells, comprising the steps of: (a) culturing an HPC comprising sample obtained from an individual in medium which does not induce differentiation of the HPCs for a first culture period to produce a pre-differentiation HPC population; (b) culturing the pre-differentiation HPC population in medium which induces differentiation of the HPCs to NK for a second culture period, wherein NK cell expansion also occurs in the second culture period. The present invention also provides a method for producing an expanded CD16⁺ NK cell population, comprising the steps of: (a) culturing an HPC comprising sample obtained from an individual in medium which does not induce differentiation of the HPCs for a first culture period to produce a pre-differentiation HPC population; and (b) culturing the pre-differentiation HPC population in medium which induces differentiation of the HPCs to NK for a second culture period; and (c) expanding said cells in vitro to produce an expanded NK cell population. For at least part of the first culture period, the HPCs may also be contacted with (cultured in the presence of) a compound which inhibits REV-ERB activity. Thus the medium which does not induce differentiation of the HPCs may comprise a compound which inhibits REV-ERB activity. Thus, the HPCs may be contacted with (cultured in the presence of) a compound which inhibits REV-ERB activity for at least part of step (a) of a method of the invention.

The invention also provides a method for increasing the number of CD16⁺ NK cells in an expanded NK cell population, comprising the steps of: (a) culturing an HPC comprising sample obtained from an individual in medium which does not induce differentiation of the HPCs for a first culture period to produce a pre-differentiation HPC population; and (b) culturing the pre-differentiation HPC population in medium which induces differentiation of the HPCs to NK for a second culture period. Step (b) typically comprises both the differentiation of the HPCs in the pre-differentiation HPC population and expansion of the resulting NK cells. Thus, the present invention provides a method for increasing the number of CD16⁺ NK cells in an expanded NK cell population, comprising the steps of: (a) culturing an HPC comprising sample obtained from an individual in medium which does not induce differentiation of the HPCs for a first culture period to produce a pre-differentiation HPC population; (b) culturing the pre-differentiation HPC population in medium which induces differentiation of the HPCs to NK for a second culture period, wherein NK cell expansion also occurs in the second culture period. The present invention also provides a method for increasing the number of CD16⁺ NK cells in an expanded NK cell population, comprising the steps of: (a) culturing an HPC comprising sample obtained from an individual in medium which does not induce differentiation of the HPCs for a first culture period to produce a pre-differentiation HPC population; and (b) culturing the pre-differentiation HPC population in medium which induces differentiation of the HPCs to NK for a second culture period; and (c) expanding said cells in vitro to produce an expanded NK cell population. For at least part of the first culture period, the HPCs may also be contacted with (cultured in the presence of) a compound which inhibits REV-ERB activity. Thus the medium which does not induce differentiation of the HPCs may comprise a compound which inhibits REV-ERB activity. Thus, the HPCs may be contacted with (cultured in the presence of) a compound which inhibits REV-ERB activity for at least part of step (a) of a method of the invention.

As described herein, the first culture period is between about 2 days to about 8 days. The HPCs may be contacted with a compound which inhibits REV-ERB activity for at least part of the first culture period, up to the entirety of the first culture period. Thus, the HPCs may be contacted with a compound which inhibits REV-ERB activity for 2, 2.5, 3, 3.5, 4, 4.5, 5, 5.5, 6, 6.5, 7, 7.5 or 8 days of the first culture period may or a period of any duration between 2 and 8 days, such as between 3 to 8 days or between 2.5 to 8 days. Preferably the HPCs may be contacted with a compound which inhibits REV-ERB activity for between about 2 days to about 6 days (e.g. 2, 2.5, 3, 3.5, 4, 4.5, 5, 5.5 or 6 days or a period of any duration between 2 and 6 days, such as between 2.5 to 6 days, between 3 to 6 days, or between about 4 to 6 days,) of the first culture period. More preferably the HPCs may be contacted with a compound which inhibits REV-ERB activity for between about 2 days to about 4 days (e.g. 2, 2.5, 3, 3.5 or 4 days or a period of any duration between 2 and 4 days, such as between about 2.5 to 4 days, or between about 3 to 4 days) of the first culture period.

Most preferably, the first culture period comprises a period of between about 0 to about 2 days, particularly about 1 day, in which a compound which inhibits REV-ERB activity is absent, followed by contacting the HPCs with the compound which inhibits REV-ERB activity for the remainder of the first culture period. Thus, a compound which inhibits REV-ERB activity may be added to the medium which does not induce differentiation of the HPCs after a period of between about 0 to about 2 days, particularly the compound which inhibits REV-ERB activity may be added to the medium which does not induce differentiation of the HPCs after about 1 day.

If the medium which does not induce differentiation of the HPCs is replaced during the first culture period, then the replacement medium which does not induce differentiation of the HPCs may comprise/not comprise compound which inhibits REV-ERB activity, depending on whether the presence of the compound which inhibits REV-ERB activity is desired at that point within the first culture period. By way of non-limiting example, if the medium which does not induce differentiation of the HPCs is replaced on day 2, but the HPCs are to be cultured with the compound which inhibits REV-ERB activity only from day 4, then the replacement medium which does not induce differentiation of the HPCs at day 2 would not include the compound which inhibits REV-ERB activity. By way of further limiting example, alternatively or in addition, if the medium which does not induce differentiation of the HPCs is replaced on day 6, and the HPCs are to be cultured with the compound which inhibits REV-ERB activity from day 4, then the replacement medium which does not induce differentiation of the HPCs at day 6 would include the compound which inhibits REV-ERB activity. The compound which inhibits REV-ERB activity may be added to the medium which does not induce differentiation of the HPCs before it is used to replace that on the HPCs, or the medium which does not induce differentiation of the HPCs may be replaced, and the compound which inhibits REV-ERB activity then added to the HPCs.

Alternatively or in addition, the pre-differentiation HPC population may be cultured in the presence of the compound which inhibits REV-ERB activity for at least part of a second culture period in a method described herein. Thus, the compound which inhibits REV-ERB activity may be comprised in the medium which does induce differentiation of the HPCs.

Accordingly, the present invention provides a method for producing an expanded population of CD16⁺ NK cells, comprising the steps of: (a) culturing an HPC comprising sample obtained from an individual in medium which does not induce differentiation of the HPCs for a first culture period to produce a pre-differentiation HPC population; and (b) culturing the pre-differentiation HPC population in medium which induces differentiation of the HPCs to NK for a second culture period. Step (b) typically comprises both the differentiation of the HPCs in the pre-differentiation HPC population and expansion of the resulting NK cells. Thus, the present invention provides a method for producing an expanded population of CD16⁺ NK cells, comprising the steps of: (a) culturing an HPC comprising sample obtained from an individual in medium which does not induce differentiation of the HPCs for a first culture period to produce a pre-differentiation HPC population; (b) culturing the pre-differentiation HPC population in medium which induces differentiation of the HPCs to NK for a second culture period, wherein NK cell expansion also occurs in the second culture period. The present invention also provides a method for producing an expanded CD16⁺ NK cell population, comprising the steps of: (a) culturing an HPC comprising sample obtained from an individual in medium which does not induce differentiation of the HPCs for a first culture period to produce a pre-differentiation HPC population; and (b) culturing the pre-differentiation HPC population in medium which induces differentiation of the HPCs to NK for a second culture period; and (c) expanding said cells in vitro to produce an expanded NK cell population. For at least part of the second culture period, the pre-differentiation HPCs may also be contacted with (cultured in the presence of) a compound which inhibits REV-ERB activity. Thus the medium which does induce differentiation of the HPCs may comprise a compound which inhibits REV-ERB activity. Thus, the pre-differentiation HPCs may be contacted with (cultured in the presence of) a compound which inhibits REV-ERB activity for at least part of step (b) of a method of the invention. This may be as an alternative or in addition to the inclusion of a compound which inhibits REV-ERB activity in step (a) as described herein.

The invention also provides a method for increasing the number of CD16⁺ NK cells in an expanded NK cell population, comprising the steps of: (a) culturing an HPC comprising sample obtained from an individual in medium which does not induce differentiation of the HPCs for a first culture period to produce a pre-differentiation HPC population; and (b) culturing the pre-differentiation HPC population in medium which induces differentiation of the HPCs to NK for a second culture period. Step (b) typically comprises both the differentiation of the HPCs in the pre-differentiation HPC population and expansion of the resulting NK cells. Thus, the present invention provides a method for increasing the number of CD16⁺ NK cells in an expanded NK cell population, comprising the steps of: (a) culturing an HPC comprising sample obtained from an individual in medium which does not induce differentiation of the HPCs for a first culture period to produce a pre-differentiation HPC population; (b) culturing the pre-differentiation HPC population in medium which induces differentiation of the HPCs to NK for a second culture period, wherein NK cell expansion also occurs in the second culture period. The present invention also provides a method for increasing the number of CD16⁺ NK cells in an expanded NK cell population, comprising the steps of: (a) culturing an HPC comprising sample obtained from an individual in medium which does not induce differentiation of the HPCs for a first culture period to produce a pre-differentiation HPC population; and (b) culturing the pre-differentiation HPC population in medium which induces differentiation of the HPCs to NK for a second culture period; and (c) expanding said cells in vitro to produce an expanded NK cell population. For at least part of the second culture period, the pre-differentiation HPCs may also be contacted with (cultured in the presence of) a compound which inhibits REV-ERB activity. Thus the medium which does induce differentiation of the HPCs may comprise a compound which inhibits REV-ERB activity. Thus, the pre-differentiation HPCs may be contacted with (cultured in the presence of) a compound which inhibits REV-ERB activity for at least part of step (b) of a method of the invention. This may be as an alternative or in addition to the inclusion of a compound which inhibits REV-ERB activity in step (a) as described herein.

As described herein, the second culture period may be between about 10 days to about 30 days. The pre-differentiation HPCs may be contacted with a compound which inhibits REV-ERB activity for at least part of the second culture period, up to the entirety of the second culture period. Thus, the pre-differentiation HPCs may be contacted with a compound which inhibits REV-ERB activity for 10, 11, 12, 13, 14, 15, 16, 17, 18, 19, 20, 21, 22, 23, 24, 25, 26, 27, 28, 29 or 30 days of the second culture period, or a period of any duration between 10 and 30 days. Preferably the pre-differentiation HPCs may be contacted with a compound which inhibits REV-ERB activity for between about 15 days to about 25 days (e.g. 15, 16, 17, 18, 19, 20, 21, 22, 23, 24 or 25 days or a period of any duration between 15 and 25 days) of the second culture period. More preferably the pre-differentiation HPCs may be contacted with a compound which inhibits REV-ERB activity for between about 18 days to about 22 days (e.g. 18, 19, 20, 21 or 22 days or a period of any duration between 18 and 22 days) of the second culture period. Most preferably, the pre-differentiation HPCs may be contacted with a compound which inhibits REV-ERB activity for between about 19 to 21 days of the second culture period, with about 20 days being particularly preferred.

Preferably a compound which inhibits REV-ERB activity is present only before differentiation of the HPCs is induced. In other words, a compound which inhibits REV-ERB activity is present only for at least part of the first culture period, and preferably not for at least part of the second culture period. Thus, a compound which inhibits REV-ERB activity is preferably present in the medium which does not induce differentiation of the HPCs, and not the medium which does induce differentiation of the HPCs. The pre-differentiation HPC population resulting from step (a) of a method of the invention may be transferred to a different culture vessel prior to step (b). This reduces the risk of contamination of the compound which inhibits REV-ERB activity in step (b) of the method.

The REV-ERB inhibitor compound may be added before the addition of the Notch ligand, concurrently with the Notch ligand or after the Notch ligand is added. In some embodiments the compound is added at multiple time points, for example when the culture medium is changed. As a non-limiting example, the compound of the invention may be added one day into the first culture period, and then added again at day five of the first culture period.

The disclosure in relation to addition of a Notch ligand and/or a compound which inhibits the activity of REV-ERB applies independently to all methods of the invention, e.g. for methods which also use a compound which alters the posttranslational modification of E4bp4 as described herein.

Any appropriate concentration of a compound which inhibits the activity of REV-ERB may be used, provided that it inhibits the action of REV-ERB as described herein and has utility in expanding an NK cell population. As a non-limiting example, in any aspect of the invention, a compound which inhibits the activity of REV-ERB may be used at a final concentration of about 2 to about 20 μM, about 2 to about 15 μM, about 5 to about 15 μM, about 5 to about 14 μM, about 4 to about 13 μM, about 5 to about 12 μM, about 5 to about 11 μM, or preferably about 2 to about 10 μM, such as about 5 to about 10 μM.

As described above, HPCs may be cultured in the presence or absence of a stromal support cell or feeder cell, or population thereof. In some preferred embodiments where a compound which inhibits the activity of REV-ERB is used, the cells are cultured in the absence of a stromal support cell or population thereof, but optionally may be cultured with an ECM or stromal cells as described herein.

The REV-ERB inhibitory compound may be added to the sample comprising HPCs within one week, within six days, within five days, within four days, within three days, within two days, within one day of commencing a method of the invention. For example, from the date of thawing and plating the HPCS, the date of isolating the HPCs in the sample, or on the same day as isolating the NK cell precursors. The date of isolating the HPCs and/or NK cell precursors is typically the same day that the sample is obtained from the patient. Preferably the REV-ERB inhibitory compound is added to the sample within five days, more preferably two days of commencing a method of the invention (e.g. from thawing and plating the HPCs or isolating the HPCs in the sample), such as on the day of commencement (day 0), or day one or two following commencement (e.g. from thawing and plating the HPCs or isolation of the HPCs). Most preferably the REV-ERB inhibitory compound of the invention is added to the sample one day post commencement (e.g. from thawing and plating the HPCs or isolation of the HPCs).

Thus, typically the REV-ERB inhibitory compound is present on or from one day post commencement (e.g. from thawing and plating the HPCs or isolation of the HPCs), until the pre-differentiation HPC population is cultured in the medium which induces differentiation of the pre-differentiation HPCs to NK cells (i.e. from day one post commencement (e.g. from thawing and plating the HPCs or isolation) until the end of step (a)/start of step (b)).

The cells (e.g. the HPCs in step (a)) may be cultured in the presence of a REV-ERB inhibitory compound for at least 6 hours, at least 12 hours, at least 24 hours, at least 30 hours, at least 36 hours, at least 42 hours, at least 48 hours, at least 54 hours, at least 60 hours, at least 72 hours, at least 4 days, at least 5 days, at least 6 days, at least 7 days, or 8 days. Typically for between 1-7 days, preferably for about 4 days.

Alternatively, these durations may be measured in terms of the number of cell passages. For example, at least one, at least two, at least three, at least four, at least five, at least ten, at least 20, or more passages of the cells (either in vivo, or cultured ex vivo or in vitro). Typically, these durations may be up to 10 passages of the cells, i.e. any number of passages between 1 and 10 (1, 2, 3, 4, 5, 6, 7, 8, 9 or 10 passages), preferably 1, 2 or 3 passages.

A method of the invention may comprise a recovery period, such that the HPCs are culture for a recovery period of between about 0 days to about 5 days before being contacted with a REV-ERB inhibitory compound and cultured for a first culture period. As described herein, the first culture period is between about 2 days to about 8 days. The HPCs may be contacted with a REV-ERB inhibitory compound for at least part of the first culture period, up to the entirety of the first culture period. Thus, the HPCs may be contacted with a REV-ERB inhibitory compound for 2, 2.5, 3, 3.5, 4, 4.5, 5, 5.5, 6, 6.5, 7, 7.5 or 8 days of the first culture period may or a period of any duration between 2 and 8 days. Preferably the HPCs may be contacted with a REV-ERB inhibitory compound for between about 2 days to about 6 days (e.g. 2, 2.5, 3, 3.5, 4, 4.5, 5, 5.5 or 6 days or a period of any duration between 2 and 6 days, such as between about 4 to 6 days) of the first culture period. More preferably the HPCs may be contacted with a REV-ERB inhibitory compound for between about 2 days to about 4 days (e.g. 2, 2.5, 3, 3.5 or 4 days or a period of any duration between 2 and 4 days) of the first culture period. A method may comprise a recovery period of between about 0 days to about 5 days, such as about 0 day to about 2 days, particularly about 1 day, in which a REV-ERB inhibitory compound is absent, followed by contacting the HPCs with the REV-ERB inhibitory compound for a first culture period. Thus, a REV-ERB inhibitory compound may be added to the medium which does not induce differentiation of the HPCs after a period of between about 0 days to about 5 days, such as about 0 days to about 2 days, particularly the REV-ERB inhibitory compound may be added to the medium which does not induce differentiation of the HPCs after about 1 day.

Methods Using Both Notch Ligands and Compounds which Inhibit REV-ERB Activity

The methods of the invention may comprise the use of both a Notch ligand (e.g. DLL4) and a compound which inhibits the activity of REV-ERB. Any and all disclosure herein relating to methods comprising the use of Notch ligands (e.g. concentrations, timings, etc.) can be combined without limitation to any and all disclosure relating to method comprising the use of compounds which inhibit REV-ERB activity (e.g. concentrations, timings, etc.).

Thus, the present invention provides a method for producing an expanded population of CD16⁺ NK cells, comprising the steps of: (a) culturing an HPC comprising sample obtained from an individual in medium which does not induce differentiation of the HPCs for a first culture period to produce a pre-differentiation HPC population; and (b) culturing the pre-differentiation HPC population in medium which induces differentiation of the HPCs to NK for a second culture period. Step (b) typically comprises both the differentiation of the HPCs in the pre-differentiation HPC population and expansion of the resulting NK cells. Thus, the present invention provides a method for producing an expanded population of CD16⁺ NK cells, comprising the steps of: (a) culturing an HPC comprising sample obtained from an individual in medium which does not induce differentiation of the HPCs for a first culture period to produce a pre-differentiation HPC population; (b) culturing the pre-differentiation HPC population in medium which induces differentiation of the HPCs to NK for a second culture period, wherein NK cell expansion also occurs in the second culture period. The present invention also provides a method for producing an expanded CD16⁺ NK cell population, comprising the steps of: (a) culturing an HPC comprising sample obtained from an individual in medium which does not induce differentiation of the HPCs for a first culture period to produce a pre-differentiation HPC population; and (b) culturing the pre-differentiation HPC population in medium which induces differentiation of the HPCs to NK for a second culture period; and (c) expanding said cells in vitro to produce an expanded NK cell population. For at least part of the first culture period, the HPCs may also be contacted with (cultured in the presence of) a compound which inhibits REV-ERB activity and a Notch ligand (e.g. DLL4). Thus, the medium which does not induce differentiation of the HPCs may comprise a compound which inhibits REV-ERB activity and a Notch ligand (e.g. DLL4). Thus, the HPCs may be contacted with (cultured in the presence of) a compound which inhibits REV-ERB activity and a Notch ligand (e.g. DLL4) for at least part of step (a) of a method of the invention.

The invention also provides a method for increasing the number of CD16⁺ NK cells in an expanded NK cell population, comprising the steps of: (a) culturing an HPC comprising sample obtained from an individual in medium which does not induce differentiation of the HPCs for a first culture period to produce a pre-differentiation HPC population; and (b) culturing the pre-differentiation HPC population in medium which induces differentiation of the HPCs to NK for a second culture period. Step (b) typically comprises both the differentiation of the HPCs in the pre-differentiation HPC population and expansion of the resulting NK cells. Thus, the present invention provides a method for increasing the number of CD16⁺ NK cells in an expanded NK cell population, comprising the steps of: (a) culturing an HPC comprising sample obtained from an individual in medium which does not induce differentiation of the HPCs for a first culture period to produce a pre-differentiation HPC population; (b) culturing the pre-differentiation HPC population in medium which induces differentiation of the HPCs to NK for a second culture period, wherein NK cell expansion also occurs in the second culture period. The present invention also provides a method for increasing the number of CD16⁺ NK cells in an expanded NK cell population, comprising the steps of: (a) culturing an HPC comprising sample obtained from an individual in medium which does not induce differentiation of the HPCs for a first culture period to produce a pre-differentiation HPC population; and (b) culturing the pre-differentiation HPC population in medium which induces differentiation of the HPCs to NK for a second culture period; and (c) expanding said cells in vitro to produce an expanded NK cell population. For at least part of the first culture period, the HPCs may also be contacted with (cultured in the presence of) a compound which inhibits REV-ERB activity and a Notch ligand (e.g. DLL4). Thus the medium which does not induce differentiation of the HPCs may comprise a compound which inhibits REV-ERB activity and a Notch ligand (e.g. DLL4). Thus, the HPCs may be contacted with (cultured in the presence of) a compound which inhibits REV-ERB activity and a Notch ligand (e.g. DLL4) for at least part of step (a) of a method of the invention.

As described herein, the first culture period is between about 2 days to about 8 days. The HPCs may be contacted with a compound which inhibits REV-ERB activity and a Notch ligand (e.g. DLL4) for at least part of the first culture period, up to the entirety of the first culture period. The HPCs may be contacted with a compound which inhibits REV-ERB activity and a Notch ligand (e.g. DLL4) for different lengths of time within the first culture period, or for the same length of time. Thus, the HPCs may be contacted with a compound which inhibits REV-ERB activity and a Notch ligand (e.g. DLL4) for a length of time independently selected from 2, 2.5, 3, 3.5, 4, 4.5, 5, 5.5, 6, 6.5, 7, 7.5 or 8 days of the first culture period may or a period of any duration between 2 and 8 days for each of the compound which inhibits REV-ERB activity and the Notch ligand (e.g. DLL4). Preferably the HPCs may be contacted with a compound which inhibits REV-ERB activity and a Notch ligand (e.g. DLL4) for a length of time independently selected from between about 2 days to about 6 days (e.g. 2, 2.5, 3, 3.5, 4, 4.5, 5, 5.5 or 6 days or a period of any duration between 2 and 6 days, such as between about 4 to 6 days) of the first culture period for each of the compound which inhibits REV-ERB activity and the Notch ligand (e.g. DLL4). More preferably the HPCs may be contacted with a compound which inhibits REV-ERB activity and a Notch ligand (e.g. DLL4) for a length of time independently selected from between about 2 days to about 4 days (e.g. 2, 2.5, 3, 3.5 or 4 days or a period of any duration between 2 and 4 days) of the first culture period for each of the compound which inhibits REV-ERB activity and the Notch ligand (e.g. DLL4).

Most preferably, the first culture period comprises a period of between about 0 to about 2 days, particularly about 1 day, in which a compound which inhibits REV-ERB activity and a Notch ligand (e.g. DLL4) are absent, followed by contacting the HPCs with the compound which inhibits REV-ERB activity and the Notch ligand (e.g. DLL4) for the remainder of the first culture period. Thus, a compound which inhibits REV-ERB activity and the Notch ligand (e.g. DLL4) be added to the medium which does not induce differentiation of the HPCs after a period of time selected independently for each of the compound which inhibits REV-ERB activity and the Notch ligand (e.g. DLL4) of between about 0 to about 2 days, particularly the compound which inhibits REV-ERB activity and the Notch ligand (e.g. DLL4) may be added to the medium which does not induce differentiation of the HPCs after about 1 day.

A method of the invention may comprise a recovery period, such that the HPCs are culture for a recovery period of between about 0 days to about 5 days before being contacted with a REV-ERB inhibitory compound and a Notch ligand (e.g. DLL4) and cultured for a first culture period. As described herein, the first culture period is between about 2 days to about 8 days. The HPCs may be contacted with a REV-ERB inhibitory compound and a Notch ligand (e.g. DLL4) for at least part of the first culture period, up to the entirety of the first culture period. Thus, the HPCs may be contacted with a REV-ERB inhibitory compound and a Notch ligand (e.g. DLL4) for 2, 2.5, 3, 3.5, 4, 4.5, 5, 5.5, 6, 6.5, 7, 7.5 or 8 days of the first culture period may or a period of any duration between 2 and 8 days. Preferably the HPCs may be contacted with a REV-ERB inhibitory compound and a Notch ligand (e.g. DLL4) for between about 2 days to about 6 days (e.g. 2, 2.5, 3, 3.5, 4, 4.5, 5, 5.5 or 6 days or a period of any duration between 2 and 6 days, such as between about 4 to 6 days) of the first culture period. More preferably the HPCs may be contacted with a REV-ERB inhibitory compound and a Notch ligand (e.g. DLL4) for between about 2 days to about 4 days (e.g. 2, 2.5, 3, 3.5 or 4 days or a period of any duration between 2 and 4 days) of the first culture period. A method may comprise a recovery period of between about 0 days to about 5 days, such as about 0 day to about 2 days, particularly about 1 day, in which a REV-ERB inhibitory compound and a Notch ligand (e.g. DLL4) are absent, followed by contacting the HPCs with the REV-ERB inhibitory compound and the Notch ligand (e.g. DLL4) for a first culture period. Thus, a REV-ERB inhibitory compound and a Notch ligand (e.g. DLL4) may be added to the medium which does not induce differentiation of the HPCs after a period of between about 0 days to about 5 days, such as about 0 days to about 2 days, particularly the REV-ERB inhibitory compound and the Notch ligand (e.g. DLL4) may be added to the medium which does not induce differentiation of the HPCs after about 1 day.

If the medium which does not induce differentiation of the HPCs is replaced during the first culture period, then the replacement medium which does not induce differentiation of the HPCs may comprise/not comprise a compound which inhibits REV-ERB activity and/or a Notch ligand (e.g. DLL4), depending on whether the presence of the compound which inhibits REV-ERB activity and/or the Notch ligand (e.g. DLL4) is desired at that point within the first culture period. By way of non-limiting example, if the medium which does not induce differentiation of the HPCs is replaced on day 2, but the HPCs are to be cultured with the compound which inhibits REV-ERB activity and/or the Notch ligand (e.g. DLL4) only from day 4, then the replacement medium which does not induce differentiation of the HPCs at day 2 would not include the compound which inhibits REV-ERB activity and/or the Notch ligand (e.g. DLL4). By way of further limiting example, alternatively or in addition, if the medium which does not induce differentiation of the HPCs is replaced on day 6, and the HPCs are to be cultured with the compound which inhibits REV-ERB activity and/or the Notch ligand (e.g. DLL4) from day 4, then the replacement medium which does not induce differentiation of the HPCs at day 6 would include the compound which inhibits REV-ERB activity and the Notch ligand (e.g. DLL4). The compound which inhibits REV-ERB activity and/or the Notch ligand (e.g. DLL4) may be added to the medium which does not induce differentiation of the HPCs before it is used to replace that on the HPCs, or the medium which does not induce differentiation of the HPCs may be replaced, and the compound which inhibits REV-ERB activity and/or the Notch ligand (e.g. DLL4) then added to the HPCs.

Alternatively or in addition, the pre-differentiation HPC population may be cultured in the presence of the compound which inhibits REV-ERB activity and the Notch ligand (e.g. DLL4) for at least part of a second culture period in a method described herein. Thus, the compound which inhibits REV-ERB activity and the Notch ligand (e.g. DLL4) may be comprised in the medium which does induce differentiation of the HPCs.

Accordingly, the present invention provides a method for producing an expanded population of CD16⁺ NK cells, comprising the steps of: (a) culturing an HPC comprising sample obtained from an individual in medium which does not induce differentiation of the HPCs for a first culture period to produce a pre-differentiation HPC population; and (b) culturing the pre-differentiation HPC population in medium which induces differentiation of the HPCs to NK for a second culture period. Step (b) typically comprises both the differentiation of the HPCs in the pre-differentiation HPC population and expansion of the resulting NK cells. Thus, the present invention provides a method for producing an expanded population of CD16⁺ NK cells, comprising the steps of: (a) culturing an HPC comprising sample obtained from an individual in medium which does not induce differentiation of the HPCs for a first culture period to produce a pre-differentiation HPC population; (b) culturing the pre-differentiation HPC population in medium which induces differentiation of the HPCs to NK for a second culture period, wherein NK cell expansion also occurs in the second culture period. The present invention also provides a method for producing an expanded CD16⁺ NK cell population, comprising the steps of: (a) culturing an HPC comprising sample obtained from an individual in medium which does not induce differentiation of the HPCs for a first culture period to produce a pre-differentiation HPC population; and (b) culturing the pre-differentiation HPC population in medium which induces differentiation of the HPCs to NK for a second culture period; and (c) expanding said cells in vitro to produce an expanded NK cell population. For at least part of the second culture period, the pre-differentiation HPCs may also be contacted with (cultured in the presence of) a compound which inhibits REV-ERB activity and a Notch ligand (e.g. DLL4). Thus the medium which does induce differentiation of the HPCs may comprise a compound which inhibits REV-ERB activity and a Notch ligand (e.g. DLL4). Thus, the pre-differentiation HPCs may be contacted with (cultured in the presence of) a compound which inhibits REV-ERB activity and a Notch ligand (e.g. DLL4) for at least part of step (b) of a method of the invention. This may be as an alternative or in addition to the inclusion of a compound which inhibits REV-ERB activity and/or a Notch ligand (e.g. DLL4) in step (a) as described herein.

The invention also provides a method for increasing the number of CD16⁺ NK cells in an expanded NK cell population, comprising the steps of: (a) culturing an HPC comprising sample obtained from an individual in medium which does not induce differentiation of the HPCs for a first culture period to produce a pre-differentiation HPC population; and (b) culturing the pre-differentiation HPC population in medium which induces differentiation of the HPCs to NK for a second culture period. Step (b) typically comprises both the differentiation of the HPCs in the pre-differentiation HPC population and expansion of the resulting NK cells. Thus, the present invention provides a method for increasing the number of CD16⁺ NK cells in an expanded NK cell population, comprising the steps of: (a) culturing an HPC comprising sample obtained from an individual in medium which does not induce differentiation of the HPCs for a first culture period to produce a pre-differentiation HPC population; (b) culturing the pre-differentiation HPC population in medium which induces differentiation of the HPCs to NK for a second culture period, wherein NK cell expansion also occurs in the second culture period. The present invention also provides a method for increasing the number of CD16⁺ NK cells in an expanded NK cell population, comprising the steps of: (a) culturing an HPC comprising sample obtained from an individual in medium which does not induce differentiation of the HPCs for a first culture period to produce a pre-differentiation HPC population; and (b) culturing the pre-differentiation HPC population in medium which induces differentiation of the HPCs to NK for a second culture period; and (c) expanding said cells in vitro to produce an expanded NK cell population. For at least part of the second culture period, the pre-differentiation HPCs may also be contacted with (cultured in the presence of) a compound which inhibits REV-ERB activity and a Notch ligand (e.g. DLL4). Thus the medium which does induce differentiation of the HPCs may comprise a compound which inhibits REV-ERB activity. Thus, the pre-differentiation HPCs may be contacted with (cultured in the presence of) a compound which inhibits REV-ERB activity and a Notch ligand (e.g. DLL4) for at least part of step (b) of a method of the invention. This may be as an alternative or in addition to the inclusion of a compound which inhibits REV-ERB activity and/or a Notch ligand (e.g. DLL4) in step (a) as described herein.

As described herein, the second culture period may be between about 10 days to about 30 days. The pre-differentiation HPCs may be contacted with a compound which inhibits REV-ERB activity and a Notch ligand (e.g. DLL4) for at least part of the second culture period, up to the entirety of the second culture period. Thus, the pre-differentiation HPCs may be contacted with a compound which inhibits REV-ERB activity and a Notch ligand (e.g. DLL4) for a length of time independently selected from 10, 11, 12, 13, 14, 15, 16, 17, 18, 19, 20, 21, 22, 23, 24, 25, 26, 27, 28, 29 or 30 days of the second culture period, or a period of any duration between 10 and 30 days for each of the compound which inhibits REV-ERB activity and the Notch ligand (e.g. DLL4). Preferably the pre-differentiation HPCs may be contacted with a compound which inhibits REV-ERB activity and a Notch ligand (e.g. DLL4) for a length of time independently selected from between about 15 days to about 25 days (e.g. 15, 16, 17, 18, 19, 20, 21, 22, 23, 24 or 25 days or a period of any duration between 15 and 25 days) of the second culture period for each of the compound which inhibits REV-ERB activity and the Notch ligand (e.g. DLL4). More preferably the pre-differentiation HPCs may be contacted with a compound which inhibits REV-ERB activity and a Notch ligand (e.g. DLL4) for a length of time independently selected from between about 18 days to about 22 days (e.g. 18, 19, 20, 21 or 22 days or a period of any duration between 18 and 22 days) of the second culture period for each of the compound which inhibits REV-ERB activity and the Notch ligand (e.g. DLL4). Most preferably, the pre-differentiation HPCs may be contacted with a compound which inhibits REV-ERB activity and a Notch ligand (e.g. DLL4) for a length of time independently selected from between about 19 to 21 days of the second culture period, with about 20 days being particularly preferred for each of the compound which inhibits REV-ERB activity and the Notch ligand (e.g. DLL4).

Preferably a compound which inhibits REV-ERB activity and a Notch ligand (e.g. DLL4) are present only before differentiation of the HPCs is induced. In other words, a compound which inhibits REV-ERB activity and a Notch ligand (e.g. DLL4) are present only for at least part of the first culture period, and preferably not for at least part of the second culture period. Thus, a compound which inhibits REV-ERB activity and a Notch ligand (e.g. DLL4) are preferably present in the medium which does not induce differentiation of the HPCs, and not the medium which does induce differentiation of the HPCs. The pre-differentiation HPC population resulting from step (a) of a method of the invention may be transferred to a different culture vessel prior to step (b). This reduces the risk of contamination of the compound which inhibits REV-ERB activity and the Notch ligand (e.g. DLL4) in step (b) of the method.

When a compound which inhibits REV-ERB activity and a Notch ligand (e.g. DLL4) are used, these may be added concurrently or in any order. Thus, the cells may be first exposed to a REV-ERB inhibitory compound and then cultured in the presence of a Notch ligand. Alternatively, the cells may be first cultured in the presence of a Notch ligand and then in the presence of a REV-ERB inhibitory compound. Alternatively, the cells may be simultaneously cultured in the presence of a REV-ERB inhibitory compound and a Notch ligand. Preferably the cells are first cultured in the presence of a REV-ERB inhibitory compound and then in the presence of a Notch ligand.

In some embodiments, the REV-ERB inhibitory compound of the invention is added in step (a) and the Notch ligand in step (b). In other embodiments, the Notch ligand is added in step (a) and the REV-EB inhibitory compound in step (b). In yet other embodiments, both the REV-ERB inhibitory compound and the Notch ligand added in step (a). In further embodiments, both the REV-ERB inhibitory compound and the Notch ligand are added in step (b). If the REV-ERB inhibitory compound and the Notch ligand added in the same stage (either step (a) or step (b)), that stage may be further divided so that: (i) the REV-ERB inhibitory compound is added before the Notch ligand; or (ii) the Notch ligand is added before the REV-ERB inhibitory compound. Alternatively, the Notch ligand and REV-ERB inhibitory compound may be added simultaneously in the same stage. Preferably the REV-ERB inhibitor and Notch ligand are both added in step (a), with the REV-ERB inhibitor added first (e.g. at day 0 or on day 2), and the Notch ligand being added later (e.g. at day 2 or 4 respectively).

Preferred embodiments of the invention comprise (i) adding the REV-ERB inhibitory compound and the Notch ligand to the sample on day 1 post-commencement (e.g. from thawing and plating the HPCs or isolation of the HPCs); (ii) adding the REV-ERB inhibitory compound to the sample on the day of commencement (e.g. from thawing and plating the HPCs or isolation of the HPCs) and adding the Notch ligand to the sample on day 1 post-commencement (e.g. from thawing and plating the HPCs or isolation of the HPCs); or (iii) adding the REV-ERB inhibitory compound to the sample on day two post-commencement (e.g. from thawing and plating the HPCs or isolation of the HPCs) and adding the Notch ligand to the sample on day four post-commencement (e.g. from thawing and plating the HPCs or isolation of the HPCs); with option (ii) being particularly preferred. As demonstrated by the inventors, these particular conditions maximise the synergy between the REV-ERB inhibition and the Notch ligand, and hence maximising the expansion of NK cells.

The present invention provides a method for producing an expanded population of CD16⁺ NK cells, comprising the steps of: (a) culturing an HPC comprising sample obtained from an individual in medium which does not induce differentiation of the HPCs for a first culture period to produce a pre-differentiation HPC population, wherein (i) a REV-ERB inhibitory compound is added on day 1 post-commencement and (ii) following about 1 day in culture in contact with the REV-ERB inhibitory compound, the HPCs are transferred to a culture vessel coated with a Notch ligand (e.g. DLL4) for a further period of about 4 days; and (b) culturing the pre-differentiation HPC population in medium which induces differentiation of the HPCs to NK for a second culture period to allow both the differentiation of the HPCs in the pre-differentiation HPC population and expansion of the resulting NK cells. Wherein the medium which induces differentiation of the HPCs to NK does not comprise either a REV-ERB inhibitory compound or a Notch ligand.

The invention also provides a method for increasing the number of CD16⁺ NK cells in an expanded NK cell population, comprising the steps of: (a) culturing an HPC comprising sample obtained from an individual in medium which does not induce differentiation of the HPCs for a first culture period to produce a pre-differentiation HPC population, wherein (i) a REV-ERB inhibitory compound is added on day 1 post-commencement and (ii) following about 1 day in culture in contact with the REV-ERB inhibitory compound, the HPCs are transferred to a culture vessel coated with a Notch ligand (e.g. DLL4) for a further period of about 4 days; and (b) culturing the pre-differentiation HPC population in medium which induces differentiation of the HPCs to NK for a second culture period to allow both the differentiation of the HPCs in the pre-differentiation HPC population and expansion of the resulting NK cells. Wherein the medium which induces differentiation of the HPCs to NK does not comprise either a REV-ERB inhibitory compound or a Notch ligand.

The present invention provides a method for producing an expanded population of CD16⁺ NK cells, comprising the steps of: (a) culturing an HPC comprising sample obtained from an individual in medium which does not induce differentiation of the HPCs for a first culture period to produce a pre-differentiation HPC population, wherein (i) a Notch ligand (e.g. DLL4) is added on day 1 post-commencement or wherein the HPCs are transferred to a culture vessel coated with said Notch ligand (e.g. DLL4) on day 1 post-commencement and (ii) following 4 days in culture in contact with the Notch ligand (e.g. DLL4), a REV-ERB inhibitory compound is added for a further period of about 24 hours; and (b) culturing the pre-differentiation HPC population in medium which induces differentiation of the HPCs to NK for a second culture period to allow both the differentiation of the HPCs in the pre-differentiation HPC population and expansion of the resulting NK cells. Wherein the medium which induces differentiation of the HPCs to NK does not comprise either a REV-ERB inhibitory compound or a Notch ligand.

The invention also provides a method for increasing the number of CD16⁺ NK cells in an expanded NK cell population, comprising the steps of: (a) culturing an HPC comprising sample obtained from an individual in medium which does not induce differentiation of the HPCs for a first culture period to produce a pre-differentiation HPC population, wherein (i) a Notch ligand (e.g. DLL4) is added on day 1 post-commencement or wherein the HPCs are transferred to a culture vessel coated with said Notch ligand (e.g. DLL4) on day 1 post-commencement and (ii) following 4 days in culture in contact with the Notch ligand (e.g. DLL4), a REV-ERB inhibitory compound is added for a further period of about 24 hours; and (b) culturing the pre-differentiation HPC population in medium which induces differentiation of the HPCs to NK for a second culture period to allow both the differentiation of the HPCs in the pre-differentiation HPC population and expansion of the resulting NK cells. Wherein the medium which induces differentiation of the HPCs to NK does not comprise either a REV-ERB inhibitory compound or a Notch ligand.

### Additional External Stimuli

Any of the methods herein, whether comprising the use of either, both or neither of a Notch ligand or a compound which inhibits REV-ERB activity may comprise the use of one or more additional external stimuli. Any and all disclosure herein relating to methods comprising the use of Notch ligands (e.g. concentrations, timings, etc.) and/or compounds which inhibit REV-ERB activity (e.g. concentrations, timings, etc.) can be combined without limitation to any and all disclosure relating to method comprising the use of any external stimulus (e.g. concentrations, timings, etc.).

Additional external stimuli, such as growth factors and/or cytokines, may be used to further enhance the production of (CD16⁺) NK cells. Non-limiting examples of suitable external stimuli include IL-7, IL-15, Flt3L, stem cell factor (SCF), thrombopoietin (TPO), granulocyte-macrophage colony-stimulating factor (GM-CSF), IL-3 and/or IL-6, or any combination thereof. Any appropriate concentration of such factors may be used. Non-limiting examples of suitable concentrations of these factors are described herein.

The inventors have surprisingly found that omitting IL-3 from the medium which induces differentiation of the HPCs to NK, i.e. the medium used in the step of culturing the pre-differentiation HPC population in medium which induces differentiation of the HPCs to NK for a second culture period (step (b) of the methods of the invention) increases the number of CD16⁺ NK cells produced. As exemplified herein, the omission of IL-3 from the medium which induces differentiation of the HPCs to NK is particularly significant when the pre-differentiation HPC population is contacted with a Notch ligand (e.g. DLL4) in step (b), and/or the HPCs in step (a) were contacted with a Notch ligand (e.g. DLL4) in step (a). Preferably, therefore, wherein a Notch ligand (e.g. DLL4) is used in a method of the invention (in step (a) and/or (b) as described herein), IL-3 is omitted from step (b) of the method. Alternatively or in addition, wherein a method of the invention (in step (a) and/or (b) as described herein) involves the use of stromal cells, IL-3 is omitted from step (b) of the method.

Accordingly, a method of the invention may omit IL-3 from: (i) the medium which induces differentiation of the HPCs to NK, i.e. the medium used in the step of culturing the pre-differentiation HPC population in medium which induces differentiation of the HPCs to NK for a second culture period (step (b) of the methods of the invention); and/or (ii) the medium which does not induce differentiation of the HPCs for a first culture period to produce a pre-differentiation HPC population, i.e. the medium used in the step of culturing an HPC comprising sample obtained from an individual in medium which does not induce differentiation of the HPCs for a first culture period to produce a pre-differentiation HPC population (step (a) of the methods of the invention). Preferably IL-3 is omitted from the medium which induces differentiation of the HPCs to NK, i.e. the medium used in the step of culturing the pre-differentiation HPC population in medium which induces differentiation of the HPCs to NK for a second culture period (step (b) of the methods of the invention); but not from the medium which does not induce differentiation of the HPCs for a first culture period to produce a pre-differentiation HPC population, i.e. the medium used in the step of culturing an HPC comprising sample obtained from an individual in medium which does not induce differentiation of the HPCs for a first culture period to produce a pre-differentiation HPC population (step (a) of the methods of the invention).

In a step of culturing an HPC comprising sample obtained from an individual in medium which does not induce differentiation of the HPCs for a first culture period to produce a pre-differentiation HPC population, the medium may comprise one or more of GM-CSF, IL-3, IL-6, Flt3, TPO and/or SCF, or any combination thereof. Typically, the exogenous cytokines added in step (a) of a method of the invention may comprise or consist of one or more of GM-CSF, IL-3, IL-6, Flt3, TPO and/or SCF, or any combination thereof. The exogenous cytokines added in step (a) of a method of the invention may comprise GM-CSF, preferably in combination with one or more of IL-3, IL-6, Flt3, TPO and/or SCF, more preferably in combination with one or more of IL-6, Flt3, TPO and/or SCF. The exogenous cytokines added in step (a) of a method of the invention may consist of GM-CSF in combination with one or more of IL-3, IL-6, Flt3, TPO and/or SCF, more preferably in combination with one or more of IL-6, Flt3, TPO and/or SCF. Preferably, the exogenous cytokines added in step (a) of a method of the invention may comprise GM-CSF, IL-3, IL-6, Flt3, TPO and SCF, more preferably GM-CSFIL-6, Flt3, TPO and SCF. More preferably, the exogenous cytokines added in step (a) of a method of the invention may consist of GM-CSF, IL-3, IL-6, Flt3, TPO and SCF, more preferably GM-CSFIL-6, Flt3, TPO and SCF.

Thus, step (a) of a method of the invention may comprise culturing an HPC comprising sample obtained from an individual in medium which does not induce differentiation of the HPCs for a first culture period to produce a pre-differentiation HPC population, wherein said medium comprises one or more of GM-CSF, IL-3, IL-6 Flt3, TPO and/or SCF, or any combination thereof. Typically the medium which does not induce differentiation of the HPCs comprises at least GM-CSF, preferably in combination with one or more of IL-3, IL-6 Flt3, TPO and/or SCF, more preferably in combination with one or more of IL-6 Flt3, TPO and/or SCF. Preferably the medium which does not induce differentiation of the HPCs comprises GM-CSF, IL-3, IL-6 Flt3, TPO and SCF. Alternatively, the medium which does not induce differentiation of the HPCs may comprise GM-CSF, IL-6 Flt3, TPO and SCF. Exemplary concentrations of these external stimuli are described herein. Typically in step (a) the HPCs are cultured in the absence of exogenous IL-15, with exogenous 11-15 being added in step (b) only.

Typically, the exogenous cytokines added in step (b) of a method of the invention may comprise of one or more of IL-7, IL-15, Flt3, and/or SCF, or any combination thereof. The exogenous cytokines added in step (b) of a method of the invention may consist of one or more of IL-7, IL-15, Flt3, and/or SCF, or any combination thereof. Preferably, the exogenous cytokines added in step (b) of a method of the invention may comprise IL-7, IL-15, Flt3, and SCF. More preferably, the exogenous cytokines added in step (b) of a method of the invention may consist of IL-7, IL-15, Flt3, and SCF.

Thus, in a step of culturing the pre-differentiation HPC population in medium which induces differentiation of the HPCs to NK for a second culture period, the medium may comprise one or more of IL-7, IL-15, Flt3, and/or SCF, or any combination thereof. Thus, step (b) of a method of the invention may comprise culturing the pre-differentiation HPC population in medium which induces differentiation of the HPCs to NK for a second culture period, wherein said medium comprises one or more of IL-7, IL-15, Flt3, and/or SCF, or any combination thereof. Preferably the medium which does induce differentiation of the HPCs comprises IL-7, IL-15, Flt3, and SCF. Exemplary concentrations of these external stimuli are described herein. Typically the medium which induces differentiation of the HPCs to NK cells does not comprise IL-3.

Whilst IL-2 is conventionally used for the production of NK cells, the present invention typically does not rely on IL-2. Typically, the medium used in step (a) and/or (b) does not comprise IL-2. Preferably, the medium used in steps (a) and (b) does not comprise IL-2, such that a method of the invention does not use IL-2 and may be described as IL-2 free.

As a non-limiting example, IL-7 may be used at a concentration of about 1 ng/ml to about 100 ng/ml, about 1 ng/ml to about 50 ng/ml, about 1 ng/ml to about 25 ng/ml, about 1 ng/ml to about 10 ng/ml or less. IL-7 may be used at a concentration of about 50 ng/ml, about 25 ng/ml, about 20 ng/ml, about 15 ng/ml, about 10 ng/ml or about 5 ng/ml, preferably about 10 ng/ml, more preferably about ng/ml.

As a non-limiting example, Flt3L may be used at a concentration of about 1 ng/ml to about 100 ng/ml, about 10 ng/ml to about 100 ng/ml, about 1 ng/ml to about 50 ng/ml, about 1 ng/ml to about 25 ng/ml, about 1 ng/ml to about 10 ng/ml or less. Flt3L may be used at a concentration of about 100 ng/ml, about 50 ng/ml, about 25 ng/ml, about 20 ng/ml, about 15 ng/ml, about 10 ng/ml or about 5 ng/ml, preferably about 100 ng/ml for the medium which does not induce differentiation, preferably about 10 ng/ml for the differentiation medium.

As a non-limiting example, SCF may be used at a concentration of about 1 ng/ml to about 200 ng/ml, about 1 ng/ml to about 150 ng/ml, about 1 ng/ml to about 100 ng/ml, about 20 ng/ml to about 100 ng/ml, about 1 ng/ml to about 50 ng/ml or less, preferably from about 20 ng/ml to about 100 ng/ml. SCF may be used at a concentration of about 150 ng/ml, about 125 ng/ml, about 120 ng/ml, about 110 ng/ml, about 100 ng/ml, about 90 ng/ml, about 80 ng/ml or about 75 ng/ml, preferably about 100 ng/ml.

As a non-limiting example, IL-15 may be used at a concentration of about 1 ng/ml to about 100 ng/ml, about 10 ng/ml to about 50 ng/ml, about 1 ng/ml to about 50 ng/ml, about 1 ng/ml to about 40 ng/ml, about 1 ng/ml to about 30 ng/ml, about 1 ng/ml to about 20 ng/ml, about 1 ng/ml to about 10 ng/ml or less, preferably from about 10 ng/ml to about 50 ng/ml. IL-15 may be used at a concentration of about 50 ng/ml, about 40 ng/ml, about 35 ng/ml, about 30 ng/ml, about 25 ng/ml, about 20 ng/ml or about 10 ng/ml, preferably about 30 ng/ml, more preferably about 10 ng/ml.

As a non-limiting example, TPO may be used at a concentration of about 1 ng/ml to about 100 ng/ml, about 1 ng/ml to about 50 ng/ml, about 1 ng/ml to about 40 ng/ml, about 1 ng/ml to about 30 ng/ml, about 1 ng/ml to about 20 ng/ml, about 1 ng/ml to about 10 ng/ml or less. TPO may be used at a concentration of about 50 ng/ml, about 40 ng/ml, about 35 ng/ml, about 30 ng/ml, about 25 ng/ml, about 20 ng/ml or about 10 ng/ml, preferably about 30 ng/ml, more preferably about 100 ng/ml.

As a non-limiting example, GM-CSF may be used at a concentration of about 1 ng/ml to about 100 ng/ml, about 1 ng/ml to about 50 ng/ml, about 1 ng/ml to about 40 ng/ml, about 1 ng/ml to about ng/ml, about 1 ng/ml to about 20 ng/ml, about 1 ng/ml to about 10 ng/ml or less. IL-15 may be used at a concentration of about 50 ng/ml, about 40 ng/ml, about 35 ng/ml, about 30 ng/ml, about ng/ml, about 20 ng/ml or about 10 ng/ml, preferably about 10 ng/ml or 30 ng/ml.

As a non-limiting example, IL-3 may be used at a concentration of about 1 ng/ml to about 100 ng/ml, about 1 ng/ml to about 50 ng/ml, about 1 ng/ml to about 40 ng/ml, about 1 ng/ml to about 30 ng/ml, about 1 ng/ml to about 20 ng/ml, about 1 ng/ml to about 10 ng/ml or less. IL-15 may be used at a concentration of about 50 ng/ml, about 40 ng/ml, about 35 ng/ml, about 30 ng/ml, about 25 ng/ml, about 20 ng/ml or about 10 ng/ml, preferably about 30 ng/ml, more preferably about 10 ng/ml.

As a non-limiting example, IL-6 may be used at a concentration of about 1 ng/ml to about 100 ng/ml, about 10 ng/ml to 50 ng/ml, about 1 ng/ml to about 50 ng/ml, about 1 ng/ml to about 40 ng/ml, about 1 ng/ml to about 30 ng/ml, about 1 ng/ml to about 20 ng/ml, about 1 ng/ml to about 10 ng/ml or less, preferably from about 10 ng/ml to 50 ng/ml. IL-6 may be used at a concentration of about 50 ng/ml, about 40 ng/ml, about 35 ng/ml, about 30 ng/ml, about 25 ng/ml, about 20 ng/ml or about 10 ng/ml, preferably about 10 ng/ml or about 30 ng/ml.

The medium which does not induce differentiation of the HPCs (i.e. the medium used in step (a) may comprise: (i) GM-CSF at a concentration of 1 ng/ml to about 100 ng/ml, preferably about ng/ml (ii) IL-3 at a concentration of about 1 ng/ml to about 100 ng/ml, preferably about 10 ng/ml (iii) IL-6 at a concentration of from about 10 ng/ml to 50 ng/ml, preferably about 10 ng/ml (iv) Flt3 at a concentration from about 10 ng/ml to about 100 ng/ml, preferably about 100 ng/ml, (v) TPO at a concentration of from about 1 ng/ml to about 100 ng/ml, preferably about 100 ng/ml and (vi) SCF at a concentration of from about 20 ng/ml to about 100 ng/ml, preferably about 100 ng/ml.

The medium which does not induce differentiation of the HPCs (i.e. the medium used in step (a) may comprise: (i) GM-CSF at a concentration of about 10 ng/ml (ii) IL-3 at a concentration of about ng/ml (iii) IL-6 at a concentration of about 10 ng/ml (iv) Flt3 at a concentration about 100 ng/ml, (v) TPO at a concentration of about 100 ng/ml and (vi) SCF at a concentration of from about 100 ng/ml.

The medium which does not induce differentiation of the HPCs (i.e. the medium used in step (a) may comprise: (i) GM-CSF at a concentration of 1 ng/ml to about 100 ng/ml, preferably about ng/ml (ii) IL-6 at a concentration of from about 10 ng/ml to 50 ng/ml, preferably about 10 ng/ml (iii) Flt3 at a concentration from about 10 ng/ml to about 100 ng/ml, preferably about 100 ng/ml, (iv) TPO at a concentration of from about 1 ng/ml to about 100 ng/ml, preferably about 100 ng/ml and (v) SCF at a concentration of from about 20 ng/ml to about 100 ng/ml, preferably about 100 ng/ml.

The medium which does not induce differentiation of the HPCs (i.e. the medium used in step (a) may comprise: (i) GM-CSF at a concentration of about 10 ng/ml, (ii) IL-6 at a concentration of about ng/ml, (iii) Flt3 at a concentration about 100 ng/ml, (iv) TPO at a concentration of about 100 ng/ml and (v) SCF at a concentration of from about 100 ng/ml.

The medium which induces differentiation of the HPCs to may comprise: (i) IL-7 at a concentration of from about 1 ng/ml to about 100 ng/ml, preferably about 20 ng/ml, (ii) IL-15 at a concentration of from about 10 ng/ml to about 50 ng/ml, preferably about 10 ng/ml, (iii) Flt3 at a concentration of from about 10 ng/ml to about 100 ng/ml, preferably about 10 ng/ml, and (iv) SCF at a concentration of from about 20 ng/ml to about 100 ng/ml, preferably about 20 ng/ml.

The medium which induces differentiation of the HPCs to may comprise: (i) IL-7 at a concentration of about 20 ng/ml, (ii) IL-15 at a concentration of about 10 ng/ml, (iii) Flt3 at a concentration of about 10 ng/ml, and (iv) SCF at a concentration of about 20 ng/ml.

The HPCs and/or the pre-differentiation HPC population may be cultured on or with suitable support/stromal cells or cell layer. Any appropriate stromal cell may be used, including, but not limited to OP9 stromal cells and/or EL08 cells (e.g. EL08-ID2 stromal cells), with EL08 cells being preferred. In other words step (a) and/or step (b) of a method of the invention may be carried out in the presence of a stromal/support cell or stromal/support cell layer, such as those described herein, particularly EL08 cells. Step (a) of a method of the invention may be carried out in the presence of a stromal/support cell or stromal/support cell layer, such as those described herein, particularly EL08 cells. Step (b) of a method of the invention may be carried out in the presence of a stromal/support cell or stromal/support cell layer, such as those described herein, particularly EL08 cells. Steps (a) and (b) of a method of the invention may be carried out in the presence of a stromal/support cell or stromal/support cell layer, such as those described herein, particularly EL08 cells. Alternatively, the HPCs and/or the pre-differentiation HPC population may be cultured in the absence of support/stromal cells or cell layer. In other words step (a) and/or step (b) of a method of the invention may be carried out in the absence of a stromal/support cell or stromal/support cell layer. When no support/stromal cells are used, the HPCs and/or the pre-differentiation HPC population may be cultured on an extracellular matrix (ECM) or ECM protein. An ECM protein may be of natural origin and purified from human or animal tissues. Alternatively, the ECM proteins may be genetically engineered recombinant proteins or synthetic in nature. The ECM proteins may be a whole protein or in the form of peptide fragments, native or engineered. Examples of defined and/or xeno-free ECM protein that may be useful in the matrix for cell culture include laminin, collagen I, collagen IV, fibronectin and vitronectin. The ECM composition may include synthetically generated peptide fragments of fibronectin or recombinant fibronectin, or a mixture of at least fibronectin and vitronectin.

One or more steps of a method of the invention, or the entirety of said method may be caried out in “xeno-free (XF)” or “animal component-free (ACF)” or “animal free” conditions. The terms XF and ACF when used in relation to a medium, an extracellular matrix, or a culture condition, refers to a medium, an ECM, or a culture condition which is essentially free from heterogeneous animal-derived components. For culturing human cells, any proteins of a non-human animal, such as mouse, would be xeno components. In certain aspects, the xeno-free matrix may be essentially free of any non-human animal-derived components. The media used in the methods of the invention (in steps (a) and/or (b)) may be “serum-free”, which refers to media with no unprocessed or unpurified serum, and accordingly can include media with purified blood-derived components or animal tissue-derived components (such as growth factors). The medium according to the present invention may contain or may not contain any alternatives to serum.

One or more steps of a method of the invention, or the entirety of said method may be caried out in “defined” conditions. The term “defined”, when used in relation to a medium, an extracellular matrix, or a culture condition, refers to a medium, an extracellular matrix, or a culture condition in which the nature and amounts of approximately all the components are known. A “chemically defined medium” refers to a medium in which the chemical nature of approximately all the ingredients and their amounts are known. These media are also called synthetic media.

Any methods of the invention, whether comprising the use of either, both or neither of a Notch ligand or a compound which inhibits REV-ERB activity, may comprise the use of one or more compound which results in the alteration of post-translational modification of E4bp4, thereby causing an increase in E4bp4 activity, as described herein. Optionally the alteration of post-translational modification of E4bp4 is a reduction in SUMOylation and/or phosphorylation of E4bp4 as described herein. In some preferred embodiments the compound which results in the alteration of post-translational modification of E4bp4: reduces SUMOylation at one or more of residues K10, K116, K219, K337 and/or K394 of E4bp4, or a residue corresponding thereto, or any combination thereof; and/or reduces phosphorylation at one or more of residues S286, S301 and/or S454 of E4bp4, or a residue corresponding thereto, or any combination thereof.

Any appropriate concentration of a compound which results in the alteration of post-translational modification of E4bp4 may be used, provided that it increases the activity of E4bp4 as described herein and has utility in expanding a (CD16⁺) NK cell population. As a non-limiting example, in any aspect of the invention, a compound which results in the alteration of post-translational modification of E4bp4 may be used at a final concentration of about 0.1 to about 20 μM, about 0.1 to about 15 μM, about 0.5 to about 15 μM, about 0.5 to about 14 μM, about 0.5 to about 12 μM, about 0.5 to about 11 μM, or about 0.5 to about 10 μM, such as from about 5 to about 10 μM. In some preferred embodiments, a compound which results in the alteration of post-translational modification of E4bp4 may be used at a final concentration of about 0.5 to about 5 μM, more preferably of about 0.5 to about 2 μM, even more preferable of about 0.5 to about 1 μM.

The REV-ERB inhibitor compound, Notch ligand, compound which alters E4bp4 post-translational modification and/or other external stimuli may be used simultaneously, separately or sequentially as described herein.

Each of the REV-ERB inhibitor compound, Notch ligand compound which alters E4bp4 post-translational modification and/or other external stimuli may independently be used as a single treatment or application or in multiple treatments or applications (in both in vitro, ex vivo or in vivo methods as described herein). For multiple applications, at least two, at least three, at least four, at least five, at least six, at least seven, at least eight, at least nine or more applications may be used. The multiple applications may be applied at any appropriate time points according to a method or treatment of the invention. By way of non-limiting example, a REV-ERB inhibitory compound of the invention, a Notch ligand, a compound which alters E4bp4 post-translational modification and/or other external stimuli may each independently be applied twice a day, once daily, every other day, once every three days or weekly. Typically the REV-ERB inhibitory compounds of the invention, the Notch ligand, the compound which alters E4bp4 post-translational modification and/or other external stimuli may independently be applied as necessary when the culture medium is changed.

The method of the invention may further comprise modulating (increasing or decreasing the expression and/or activity of one or more additional gene and/or protein in the HPCs in order to enhance (CD16⁺) NK cell expansion. This modulation may be elicited by a compound of the invention, including the same compound of the invention as used to inhibit the activity of REV-ERB. Alternatively, one or more additional compounds may be used to modulate the expression and/or activity of the one or more additional gene and/or protein. Said modulation may occur directly or indirectly. Indirect modulation encompasses downstream effects caused by a compound of the invention inhibiting the activity of REV-ERB.

### Therapeutic Indications

As described herein, the invention provides for the production of CD16⁺ NK cells and expanded NK cell populations with increased numbers of CD16⁺ NK cells. Also as described herein, CD16⁺ NK cells are typically more active than CD16⁻ or CD16^{lo} cells. In particular, CD16⁺ NK cells and expanded NK cell populations made by the methods of the present invention typically exhibit at least 50% greater ADCC, preferably at least 70% greater ADCC, compared with control NK cells as described herein.

As such, the invention further relates to the therapeutic use of CD16⁺ NK cells and expanded (CD16⁺) NK cell populations, and to the therapeutic use of compositions comprising CD16⁺ NK cells and expanded (CD16⁺) NK cell populations.

Accordingly, the invention provides CD16⁺ NK cells, expanded (CD16⁺) NK cell populations and/or compositions comprising the same for use in a method of therapy.

The invention further provides a method of treatment comprising administering a therapeutically effective amount of CD16⁺ NK cells, expanded (CD16⁺) NK cell populations and/or compositions comprising the same to a patient in need thereof.

The invention further provides the use of CD16⁺ NK cells, expanded (CD16⁺) NK cell populations and/or compositions of the invention in the manufacture of a medicament.

Typically the method of therapy comprises administering the products (as described herein) to a patient or subject.

Administration of CD16⁺ NK cells, expanded (CD16⁺) NK cell populations and/or compositions according to the invention to a patient typically increases the number of NK cells in said patient. As used herein, the term “increasing the number of NK cells” can be understood to mean that the products of the invention elicit(s) a significant increase in the number of NK cells in a patient. This increase in NK cell number may be measured relative to a control (as described herein).

A reference to an increase in the number of NK cells may be quantified in terms of a fold increase relative to a control. Typically a therapeutic application of the invention can increase the number of NK cells, of at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 3 fold or more relative to a control.

Alternatively, a reference to increasing the number of NK cells may be understood to mean that, the number of NK cells is increased by at least 10%, at least 20%, at least 30%, at least 40%, at least 50%, at least 60, at least 70%, at least 80%, at least 85%, at least 90%, at least 95%, at least 100%, at least 150%, at least 200%, at least 300% or more compared with the control. Typically the number of NK cells is increased by at least 50%, preferably at least 70%, more preferably at least 80%, even more preferably at least 90% or more compared with a control.

In some embodiments, an increase in the number of NK cells may be defined in terms of the absolute number of NK cells in a sample or patient, such as the percentage of NK cells, for example the percentage of NK cells in the circulating lymphocyte population. For example, a therapy of the invention may cause an increase in NK number, resulting in a percentage of NK cells of at least 10%, at least 15%, at least 20%, at least 25%, at least 30%, at least 35%, at least 40%, at least 45%, at least 50%, at least 55%, at least 60%, at least 65%, at least 70%, at least 75%, at least 80% or more.

The number of NK cells may be determined by quantitative and/or qualitative analysis, and may be measured directly or indirectly. The number of NK cells relative to a control may be determined using any appropriate technique. Suitable standard techniques, such as flow cytometry, FACS and MACS, are known in the art.

The number of NK cells may be increased compared with a control for at least 6 hours, at least 12 hours, at least 24 hours, at least 30 hours, at least 36 hours, at least 42 hours, at least 48 hours, at least 54 hours, at least 60 hours, at least 72 hours, at least 4 days, at least 5 days, at least 6 days, at least 1 week, at least 2 weeks, at least 3 weeks, at least 1 month or more. Typically this is assessed relative to the last administration of the treatment.

The number of NK cells may be quantified in terms of the total number of NK cells in a sample from an individual/patient or culture sample (from an ex vivo method of the invention).

In the context of the therapeutic uses and methods of the invention, a “subject” or “patient” (these terms are used interchangeably herein) is any animal patient that would benefit from an increase in the number of NK cells. Typical animal patients are mammals, such as primates. Preferably the patient is a human.

The therapeutic use or method of the invention may comprise administering a therapeutically effective amount of CD16⁺ NK cells, expanded (CD16⁺) NK cell populations and/or compositions (as defined herein), either alone or in combination with other therapeutic agents, to a subject or individual.

As used herein, the term “treatment” or “treating” embraces therapeutic or preventative/prophylactic measures.

The compounds or products of the invention may also be used as a preventative therapy. As used herein, the term “preventing” includes preventing the onset of symptoms associated with a disease or disorder that may be treated by increasing NK cell number and/or reducing the severity or intensity of said symptoms. The term “preventing” includes inducing or providing protective immunity against such diseases or disorders, particularly infectious diseases as described herein. Immunity may be quantified using any appropriate technique, examples of which are known in the art.

CD16⁺ NK cells, expanded (CD16⁺) NK cell populations and/or compositions of the invention may be administered to a patient already having a disease or disorder which may be treated by increasing NK cell number. For example, the patient may be suspected of having an infectious disease or cancer as described herein, and may or may not be showing symptoms of said disease or disorder. When administered to such a patient, a compound or products of the invention can cure, delay, reduce the severity of, or ameliorate one or more symptoms, and/or prolong the survival of a subject beyond that expected in the absence of such treatment.

Alternatively, CD16⁺ NK cells, expanded (CD16⁺) NK cell populations and/or compositions of the invention may be administered to a patient who may ultimately be infected with a particular infectious disease, or develop a disease or disorder as described herein, in order to cure, delay, reduce the severity of, or ameliorate one or more symptoms, and/or prolong the survival of a subject beyond that expected in the absence of such treatment, or, in the case of infectious diseases help prevent that patient from transmitting said disease.

The treatments and preventative therapies of the present invention are applicable to a variety of different subjects of different ages. In the context of humans, the therapies are applicable to children (e.g. infants, children under 5 years old, older children or teenagers) and adults. In the context of other animal subjects (e.g. mammals such as primates), the therapies are applicable to immature subjects and mature/adult subjects.

The invention relates to the treatment of any disease or disorder which may be beneficially treated with by increasing the number of NK cells in a patient. Such diseases and disorders include cancer, infectious diseases (acute and chronic), autoimmune diseases and diseases or disorders related to female infertility or pregnancy. Infectious diseases that may be treated according to the present invention include viral infection, and infection by other pathogens, including bacteria, protists, fugal, or helminth pathogens. Typically said pathogens are intracellular pathogens which have at least one intracellular phase in their life cycle. Infections of particular interest include viral infections, and zoonotic infections that are of particular importance from a public health perspective. Cancers that may be treated according to the present invention include bladder cancer, blood cancers, leukaemia, bone cancers, bowel cancer, brain tumours, breast cancer, kidney cancer, liver cancer, lung cancer, melanoma, ovarian cancer, pancreatic cancer, prostate cancer, skin cancer, stomach cancer, testicular cancer and uterine cancer. Autoimmune diseases that may be treated according to the present invention include systemic lupus erythematosus, rheumatoid arthritis, multiple sclerosis and obesity-induced insulin resistance. As used herein, the term diseases or disorders related to female infertility or pregnancy includes, but is not limited to, fetal growth restriction, preterm labour, defects in uterine vascular remodelling and preeclampsia.

The CD16⁺ NK cells, expanded (CD16⁺) NK cell populations and/or compositions of the invention may be used in combination with one or more additional therapeutic agents or treatments, which typically may be selected from a conventional treatment for the disease or disorder to be treated. As a non-limiting example, if CD16⁺ NK cells, expanded (CD16⁺) NK cell populations and/or compositions of the invention are for use in the treatment of a cancer, such as lung cancer, then said CD16⁺ NK cells, expanded (CD16⁺) NK cell populations and/or compositions may be used in combination with conventional treatments for lung cancer, such as radiotherapy, chemotherapy or surgery. When used in combination with one or more additional therapeutic agent or treatment, CD16⁺ NK cells, expanded (CD16⁺) NK cell populations and/or compositions of the invention may be administered before, simultaneously with, or after the administration of the one or more additional therapeutic agent or treatment.

In some preferred embodiments, CD16⁺ NK cells, expanded (CD16⁺) NK cell populations and/or compositions of the invention is for use in combination with antibody-mediated immunotherapy. Antibody-mediated immunotherapy involves the administration of antibodies to a patient to target disease-specific antigens. Such antibodies could be used to increase the specificity and killing activity of NK cells, which express receptors for the Fc regions of IgG antibodies. Activation of these Fc receptors, leads to NK cell activation, resulting in cytokine secretion and release of cytotoxic granules by the activated NK cell, causing lysis of the cell expressing the disease antigen. Such combination therapy is particularly preferred for the treatment of cancer (using antibodies to tumour-specific antigens). Any antibody used in immunotherapy may be used in combination with CD16⁺ NK cells, expanded (CD16⁺) NK cell populations and/or compositions of the invention. Non-limiting examples of such antibodies include anti-CD20 mAbs (non-Hodgkin's lymphoma, chronic lymphocytic lymphoma), anti-ganglioside D2 (anti-GD2) mAbs (neuroblastoma, melanoma), anti-human epidermal growth factor (anti-HER2) mAbs (breast and gastric cancers), anti-epidermal growth factor receptor (anti-EGFR) mAbs (colorectal and head and neck cancer).

### Pharmaceutical Compositions and Formulations

The terms “compound” or “products” are herein used interchangeably with the terms “therapeutic/prophylactic composition”, “formulation” or “medicament”.

The compound, products or expanded (CD16⁺) NK cell population of the invention (as defined above) can be combined or administered in addition to a pharmaceutically acceptable carrier, diluent and/or excipient. Alternatively or in addition the compound, products or expanded (CD16⁺) NK cell population of the invention can further be combined with one or more of a salt, excipient, diluent, adjuvant, immunoregulatory agent and/or antimicrobial compound.

Pharmaceutically acceptable salts include acid addition salts formed with inorganic acids such as, for example, hydrochloric or phosphoric acids, or with organic acids such as acetic, oxalic, tartaric, maleic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Administration of immunogenic compositions, therapeutic formulations, medicaments and prophylactic formulations is generally by conventional routes e.g. intravenous, subcutaneous, intraperitoneal, or mucosal routes. The administration may be by parenteral injection, for example, a subcutaneous, intradermal or intramuscular injection. For example, formulations comprising antibodies or expanded NK cell populations of the invention may be particularly suited to administration intravenously, intramuscularly, intradermally, or subcutaneously. Administration of small molecule REV-ERB inhibitors may be injection, such as intravenously, intramuscularly, intradermally, or subcutaneously, or by oral administration (small molecules with molecule weight of less than 500 Da typically exhibiting oral bioavailability).

Accordingly, immunogenic compositions, therapeutic formulations, medicaments and prophylactic formulations of the invention may be prepared as injectables, either as liquid solutions or suspensions. Solid forms suitable for solution in, or suspension in, liquid prior to injection may alternatively be prepared. The preparation may also be emulsified, or the peptide encapsulated in liposomes or microcapsules.

The active immunogenic ingredients (such as the compounds, products or expanded (CD16⁺) NK cell populations of the invention) are often mixed with excipients which are pharmaceutically acceptable and compatible with the active ingredient. Suitable excipients are, for example, water, saline, dextrose, glycerol, ethanol, or the like and combinations thereof. In addition, if desired, the vaccine may contain minor amounts of auxiliary substances such as wetting or emulsifying agents, pH buffering agents, and/or adjuvants which enhance the effectiveness of the composition.

Generally, the carrier is a pharmaceutically-acceptable carrier. Non-limiting examples of pharmaceutically acceptable carriers include water, saline, and phosphate-buffered saline. In some embodiments, however, where the composition comprises a compound or products of the invention, this may be in lyophilized form, in which case it may include a stabilizer, such as BSA. In some embodiments, it may be desirable to formulate the composition with a preservative, such as thiomersal or sodium azide, to facilitate long term storage.

Examples of additional adjuvants which may be effective include but are not limited to: complete Freunds adjuvant (CFA), Incomplete Freunds adjuvant (IFA), Saponin, a purified extract fraction of Saponin such as Quil A, a derivative of Saponin such as QS-21, lipid particles based on Saponin such as ISCOM/ISCOMATRIX, E. coli heat labile toxin (LT) mutants such as LTK63 and/or LTK72, aluminium hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryl oxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion, the MF59 formulation developed by Novartis, and the AS02, ASO1, AS03 and AS04 adjuvant formulations developed by GSK Biologicals (Rixensart, Belgium).

Examples of buffering agents include, but are not limited to, sodium succinate (pH 6.5), and phosphate buffered saline (PBS; pH 6.5 and 7.5).

Additional formulations which are suitable for other modes of administration include suppositories and, in some cases, oral formulations or formulations suitable for distribution as aerosols. For suppositories, traditional binders and carriers may include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders.

The dosage ranges for administration of the compounds or products of the present invention are those which produce the desired therapeutic effect. It will be appreciated that the dosage range required depends on the precise nature of the compound or products, the route of administration, the nature of the formulation, the age of the patient, the nature, extent or severity of the patient's condition, contraindications, if any, and the judgement of the attending physician. Variations in these dosage levels can be adjusted using standard empirical routines for optimisation. Similarly, the dose of a compound or products of the invention for use in a method of the invention, particularly an ex vivo method, can be readily determined by one of skill in the art, and is any dose that produces the desired increase in NK cell number and/or elicits the desired expansion in NK cells, to produce an expanded NK cell population. As a non-limiting example, doses of SR8278 according to the present invention may give rise to a final concentration of about 2 to about 20 μM, about 2 to about 15 μM, about 5 to about 15 μM, about 5 to about 14 μM, about 4 to about 13 μM, about 5 to about 12 μM, about 5 to about 11 μM, or preferably about 5 to about 10 μM.

The invention also provides the use of an expanded (CD16⁺) NK cell population (as described herein) in a pharmaceutical formulation. Any and all of the disclosure herein in relation to formulations of a compound of the invention may apply equally and independently to therapeutic applications of the expanded (CD16⁺) NK cell populations of the invention.

### Sequence Information

### Key to SEQ ID NOs

SEQ ID NO: 1—E4bp4 mRNA sequence (X64318.1)
SEQ ID NO: 2—E4bp4 amino acid sequence (X64318.1)
SEQ ID NO: 3—REV-ERBα mRNA sequence (NM_021724.4)
SEQ ID NO: 4—REV-ERBα amino acid sequence (NM_021724.4)
SEQ ID NO: 5—REV-ERBβ mRNA sequence (AB307693.1)
SEQ ID NO: 6—REV-ERBβ amino acid sequence (AB307693.1)
SEQ ID NO: 7—Delta-like ligand 4 mRNA sequence (AF253468.1)
SEQ ID NO: 8—Delta-like ligand 4 amino acid sequence (AF253468.1)
SEQ ID NO: 9—Human Notch1 cDNA sequence (CR457221.1)
SEQ ID NO: 10—Human Notch1 protein sequence (CR457221.1)

### Key to Sequences

```
E4bp4 gene sequence (X64318.1)
SEQ ID NO: 1
      1 gcccctttct ttctcctcgt cggcccgaga gcaggaacac gataacgaag gaggcccaac
    61 ttcattcaat aaggagcctg acggatttat cccagacggt agaacaaaag gaagaatatt
  121 gatggatttt aaaccagagt ttttaaagag cttgagaata cggggaaatt aatttgttct
  181 cctacacaca tagatagggt aaggttgttt ctgatgcagc tgagaaaaat gcagaccgtc
  241 aaaaaggagc aggcgtctct tgatgccagt agcaatgtgg acaagatgat ggtccttaat
  301 tctgctttaa cggaagtgtc agaagactcc acaacaggtg aggacgtgct tctcagtgaa
  361 ggaagtgtgg ggaagaacaa atcttctgca tgtcggagga aacgggaatt cattcctgat
  421 gaaaagaaag atgctatgta ttgggaaaaa aggcggaaaa ataatgaagc tgccaaaaga
  481 tctcgtgaga agcgtcgact gaatgacctg gttttagaga acaaactaat tgcactggga
  541 gaagaaaacg ccactttaaa agctgagctg ctttcactaa aattaaagtt tggtttaatt
  601 agctccacag catatgctca agagattcag aaactcagta attctacagc tgtgtacttt
  661 caagattacc agacttccaa atccaatgtg agttcatttg tggacgagca cgaaccctcg
  721 atggtgtcaa gtagttgtat ttctgtcatt aaacactctc cacaaagctc gctgtccgat
  781 gtttcagaag tgtcctcagt agaacacacg caggagagct ctgtgcaggg aagctgcaga
  841 agtcctgaaa acaagttcca gattatcaag caagagccga tggaattaga gagctacaca
  901 agggagccaa gagatgaccg aggctcttac acagcgtcca tctatcaaaa ctatatgggg
  961 aattctttct ctgggtactc acactctccc ccactactgc aagtcaaccg atcctccagc
1021 aactccccga gaacgtcgga aactgatgat ggtgtggtag gaaagtcatc tgatggagaa
1081 gacgagcaac aggtccccaa gggccccatc cattctccag ttgaactcaa gcatgtgcat
1141 gcaactgtgg ttaaagttcc agaagtgaat tcctctgcct tgccacacaa gctccggatc
1201 aaagccaaag ccatgcagat caaagtagaa gcctttgata atgaatttga ggccacgcaa
1261 aaactttcct cacctattga catgacatct aaaagacatt tcgaactcga aaagcatagt
1321 gccccaagta tggtacattc ttctcttact cctttctcag tgcaagtgac taacattcaa
1381 gattggtctc tcaaatcgga gcactggcat caaaaagaac tgagtggcaa aactcagaat
1441 agtttcaaaa ctggagttgt tgaaatgaaa gacagtggct acaaagtttc tgacccagag
1501 aacttgtatt tgaagcaggg gatagcaaac ttatctgcag aggttgtctc actcaagaga
1561 cttatagcca cacaaccaat ctctgcttca gactctgggt aaattactac tgagtaagag
1621 ctgggcattt agaaagatgt catttgcaat agagcagtcc attttgtatt atgctgaatt
1681 ttcactggac ctgtgatgtc atttcactgt gatgtgcaca tgttgtctgt ttggtgtctt
1741 tttgtgcaca gattatgatg aagattagat tgtgttatca ctctgcctgt gtatagtcag
1801 atagtcatat gcgtaaggct gtatatatta agnttttatt tttgttgttc tattataaag
1861 tgtgtaagtt accagtttca ataaaggatt ggtgacaaac acagaaaaaa aaaaaaaaaa
1921 aaa
E4bp4 amino acid sequence (X64318.1)
SEQ ID NO: 2
MQLRKMQTVKKEQASLDASSNVDKMMVLNSALTEVSEDSTTGEDVLLSEGSVGKNKSSACRRKREFIPDEKKDAM
YWEKRRKNNEAAKRSREKRRLNDLVLENKLIALGEENATLKAELLSLKLKFGLISSTAYAQEIQKLSNSTAVYFQ
DYQTSKSNVSSFVDEHEPSMVSSSCISVIKHSPQSSLSDVSEVSSVEHTQESSVQGSCRSPENKFQIIKQEPMEL
ESYTREPRDDRGSYTASIYQNYMGNSFSGYSHSPPLLQVNRSSSNSPRTSETDDGVVGKSSDGEDEQQVPKGPIH
SPVELKHVHATVVKVPEVNSSALPHKLRIKAKAMQIKVEAFDNEFEATQKLSSPIDMTSKRHFELEKHSAPSMVH
SSLTPFSVQVTNIQDWSLKSEHWHQKELSGKTQNSFKTGVVEMKDSGYKVSDPENLYLKQGIANLSAEVVSLKRL
IATQPISASDSG
REV-ERBa mRNA sequence (NM_021724.4)
SEQ ID NO: 3
      1 gggcacgagg cgctccctgg gatcacatgg tacctgctcc agtgccgcgt gcggcccggg
    61 aaccctgggc tgctggcgcc tgcgcagagc cctctgtccc agggaaaggc tcgggcaaaa
  121 ggcggctgag attggcagag tgaaatatta ctgccgaggg aacgtagcag ggcacacgtc
  181 tcgcctcttt gcgactcggt gccccgtttc tccccatcac ctacttactt cctggttgca
  241 acctctcttc ctctgggact tttgcaccgg gagctccaga ttcgccaccc cgcagcgctg
  301 cggagccggc aggcagaggc accccgtaca ctgcagagac ccgaccctcc ttgctacctt
  361 ctagccagaa ctactgcagg ctgattcccc ctacacactc tctctgctct tcccatgcaa
  421 agcagaactc cgttgcctca acgtccaacc cttctgcagg gctgcagtcc ggccacccca
  481 agaccttgct gcagggtgct tcggatcctg atcgtgagtc gcggggtcca ctccccgccc
  541 ttagccagtg cccagggggc aacagcggcg atcgcaacct ctagtttgag tcaaggtcca
  601 gtttgaatga ccgctctcag ctggtgaaga catgacgacc ctggactcca acaacaacac
  661 aggtggcgtc atcacctaca ttggctccag tggctcctcc ccaagccgca ccagccctga
  721 atccctctat agtgacaact ccaatggcag cttccagtcc ctgacccaag gctgtcccac
  781 ctacttccca ccatccccca ctggctccct cacccaagac ccggctcgct cctttgggag
  841 cattccaccc agcctgagtg atgacggctc cccttcttcc tcatcttcct cgtcgtcatc
  901 ctcctcctcc ttctataatg ggagcccccc tgggagtcta caagtggcca tggaggacag
  961 cagccgagtg tcccccagca agagcaccag caacatcacc aagctgaatg gcatggtgtt
1021 actgtgtaaa gtgtgtgggg acgttgcctc gggcttccac tacggtgtgc acgcctgcga
1081 gggctgcaag ggctttttcc gtcggagcat ccagcagaac atccagtaca aaaggtgtct
1141 gaagaatgag aattgctcca tcgtccgcat caatcgcaac cgctgccagc aatgtcgctt
1201 caagaagtgt ctctctgtgg gcatgtctcg agacgctgtg cgttttgggc gcatccccaa
1261 acgagagaag cagcggatgc ttgctgagat gcagagtgcc atgaacctgg ccaacaacca
1321 gttgagcagc cagtgcccgc tggagacttc acccacccag caccccaccc caggccccat
1381 gggcccctcg ccaccccctg ctccggtccc ctcacccctg gtgggcttct cccagtttcc
1441 acaacagctg acgcctccca gatccccaag ccctgagccc acagtggagg atgtgatatc
1501 ccaggtggcc cgggcccatc gagagatctt cacctacgcc catgacaagc tgggcagctc
1561 acctggcaac ttcaatgcca accatgcatc aggtagccct ccagccacca ccccacatcg
1621 ctgggaaaat cagggctgcc cacctgcccc caatgacaac aacaccttgg ctgcccagcg
1681 tcataacgag gccctaaatg gtctgcgcca ggctccctcc tcctaccctc ccacctggcc
1741 tcctggccct gcacaccaca gctgccacca gtccaacagc aacgggcacc gtctatgccc
1801 cacccacgtg tatgcagccc cagaaggcaa ggcacctgcc aacagtcccc ggcagggcaa
1861 ctcaaagaat gttctgctgg catgtcctat gaacatgtac ccgcatggac gcagtgggcg
1921 aacggtgcag gagatctggg aggatttctc catgagcttc acgcccgctg tgcgggaggt
1981 ggtagagttt gccaaacaca tcccgggctt ccgtgacctt tctcagcatg accaagtcac
2041 cctgcttaag gctggcacct ttgaggtgct gatggtgcgc tttgcttcgt tgttcaacgt
2101 gaaggaccag acagtgatgt tcctaagccg caccacctac agcctgcagg agcttggtgc
2161 catgggcatg ggagacctgc tcagtgccat gttcgacttc agcgagaagc tcaactccct
2221 ggcgcttacc gaggaggagc tgggcctctt caccgcggtg gtgcttgtct ctgcagaccg
2281 ctcgggcatg gagaattccg cttcggtgga gcagctccag gagacgctgc tgcgggctct
2341 tcgggctctg gtgctgaaga accggccctt ggagacttcc cgcttcacca agctgctgct
2401 caagctgccg gacctgcgga ccctgaacaa catgcattcc gagaagctgc tgtccttccg
2461 ggtggacgcc cagtgacccg cccggccggc cttctgccgc tgcccccttg tacagaatcg
2521 aactctgcac ttctctctcc tttacgagac gaaaaggaaa agcaaaccag aatcttattt
2581 atattgttat aaaatattcc aagatgagcc tctggccccc tgagccttct tgtaaatacc
2641 tgcctccctc ccccatcacc gaacttcccc tcctccccta tttaaaccac tctgtctccc
2701 ccacaaccct cccctggccc tctgatttgt tctgttcctg tctcaaatcc aatagttcac
2761 agctgagctg gcttcaaaaa aaaaaaaaaa aaa
REV-ERBa amino acid sequence (NM_021724.4)
SEQ ID NO: 4
MTTLDSNNNTGGVITYIGSSGSSPSRTSPESLYSDNSNGSFQSLTQGCPTYFPPSPTGSLTQDPARSFGSIPPSL
SDDGSPSSSSSSSSSSSSFYNGSPPGSLQVAMEDSSRVSPSKSTSNITKLNGMVLLCKVCGDVASGFHYGVHACE
GCKGFFRRSIQQNIQYKRCLKNENCSIVRINRNRCQQCRFKKCLSVGMSRDAVRFGRIPKREKQRMLAEMQSAMN
LANNQLSSQCPLETSPTQHPTPGPMGPSPPPAPVPSPLVGFSQFPQQLTPPRSPSPEPTVEDVISQVARAHREIF
TYAHDKLGSSPGNFNANHASGSPPATTPHRWENQGCPPAPNDNNTLAAQRHNEALNGLRQAPSSYPPTWPPGPAH
HSCHQSNSNGHRLCPTHVYAAPEGKAPANSPRQGNSKNVLLACPMNMYPHGRSGRTVQEIWEDESMSFTPAVREV
VEFAKHIPGFRDLSQHDQVTLLKAGTFEVLMVRFASLFNVKDQTVMFLSRTTYSLQELGAMGMGDLLSAMFDFSE
KLNSLALTEEELGLFTAVVLVSADRSGMENSASVEQLQETLLRALRALVLKNRPLETSRFTKLLLKLPDLRTLNN
MHSEKLLSFRVDAQ
REV-ERBB mRNA sequence (AB307693.1)
SEQ ID NO: 5
      1 atggaggtga atgcaggagg tgtgattgcc tatatcagtt cttccagctc agcctcaagc
    61 cctgcctctt gtcacagtga gggttctgag aatagtttcc agtcctcctc ctcttctgtt
  121 ccatcttctc caaatagctc taattctgat accaatggta atcccaagaa tggtgatctc
  181 gccaatattg aaggcatctt gaagaatgat cgaatagatt gttctatgaa aacaagcaaa
  241 tcgagtgcac ctgggatgac aaaaaatcat agtggtgtga caaaatttag tggcatggtt
  301 ctactgtgta aagtctgtgg ggatgtggcg tcaggattcc actatggagt tcatgcttgc
  361 gaaggctgta agggtttctt tcggagaagt attcaacaaa acatccagta caagaagtgc
  421 ctgaagaatg aaaactgttc tataatgaga atgaatagga acagatgtca gcaatgtcgc
  481 ttcaaaaagt gtctgtctgt tggaatgtca agagatgctg ttcggtttgg tcgtattcct
  541 aagcgtgaaa aacagaggat gctaattgaa atgcaaagtg caatgaagac catgatgaac
  601 agccagttca gtggtcactt gcaaaatgac acattagtag aacatcatga acagacagcc
  661 ttgccagccc aggaacagct gcgacccaag ccccaactgg agcaagaaaa catcaaaagc
  721 tcttctcctc catcttctga ttttgcaaag gaagaagtga ttggcatggt gaccagagct
  781 cacaaggata cctttatgta taatcaagag cagcaagaaa actcagctga gagcatgcag
  841 ccccagagag gagaacggat tcccaagaac atggagcaat ataatttaaa tcatgatcat
  901 tgcggcaatg ggcttagcag ccattttccc tgtagtgaga gccagcagca tctcaatgga
  961 cagttcaaag ggaggaatat aatgcattac ccanatggcc atgccatttg tattgcaaat
1021 ggacattgta tgaacttctc caatgcttat actcaaagag tatgtgatag agttccgata
1081 gatggatttt ctcagaatga gaacaagaat agttacctgt gcaacactgg aggaagaatg
1141 catctggttt gtccaatgag taagtctcca tatgtggatc ctcataaatc aggacatgaa
1201 atctgggaag aattttcgat gagcttcact ccagcagtga aagaagtggt ggaatttgca
1261 aagcgtattc ctgggttcag agatctctct cagcatgacc aggtcaacct tttaaaggct
1321 gggacttttg aggttttaat ggtacggttc gcatcattat ttgatgcaaa ggaacgtact
1381 gtcacctttt taagtggaaa gaaatatagt gtggatgatt tacactcaat gggagcaggg
1441 gatctgctaa actctatgtt tgaatttagt gagaagctaa atgccctcca acttagtgat
1501 gaagagatga gtttgtttac agctgttgtc ctggtatctg cagatcgatc tggaatagaa
1561 aacgtcaact ctgtggaggc tttgcaggaa actctcattc gtgcactaag gaccttaata
1621 atgaaaaacc atccaaatga ggcctctatt tttacaaaac tgcttctaaa gttgccagat
1681 cttcgatctt taaacaacat gcactctgag gagctcttgg cctttaaagt tcacccttaa
REV-ERBB amino acid sequence (AB307693.1)
SEQ ID NO: 6
MEVNAGGVIAYISSSSSASSPASCHSEGSENSFQSSSSSVPSSPNSSNSDTNGNPKNGDLANIEGILKNDRIDCS
MKTSKSSAPGMTKNHSGVTKFSGMVLLCKVCGDVASGFHYGVHACEGCKGFFRRSIQQNIQYKKCLKNENCSIMR
MNRNRCQQCRFKKCLSVGMSRDAVRFGRIPKREKQRMLIEMQSAMKTMMNSQFSGHLQNDTLVEHHEQTALPAQE
QLRPKPQLEQENIKSSSPPSSDFAKEEVIGMVTRAHKDTFMYNQEQQENSAESMQPQRGERIPKNMEQYNLNHDH
CGNGLSSHFPCSESQQHLNGQFKGRNIMHYPXGHAICIANGHCMNFSNAYTQRVCDRVPIDGESQNENKNSYLCN
TGGRMHLVCPMSKSPYVDPHKSGHEIWEEFSMSFTPAVKEVVEFAKRIPGFRDLSQHDQVNLLKAGTFEVLMVRE
ASLFDAKERTVTFLSGKKYSVDDLHSMGAGDLLNSMFEFSEKLNALQLSDEEMSLFTAVVLVSADRSGIENVNSV
EALQETLIRALRTLIMKNHPNEASIFTKLLLKLPDLRSLNNMHSEELLAFKVHP
Delta-like ligand 4 mRNA sequence (AF253468.1)
SEQ ID NO: 7
      1 atggcggcag cgtcccggag cgcctctggc tgggcgctac tgctgctggt ggcactttgg
    61 cagcagcgcg cggccggctc cggcgtcttc cagctgcagc tgcaggagtt catcaacgag
  121 cgcggcgtac tggccagtgg gggccttgc gagcccggct gccggacttt cttccgcgtc
  181 tgccttaagc acttccaggc ggtcgtctcg cccggaccct gcaccttcgg gaccgtctcc
  241 acgccggtat tgggcaccaa ctccttcgct gtccgggacg acagtagcgg cggggggcgc
  301 aaccctctcc aactgccctt caatttcacc tggccgggta ccttctcgct catcatcgaa
  361 gcttggcacg cgccaggaga cgacctgcgg ccagaggcct tgccaccaga tgcactcatc
  421 agcaagatcg ccatccaggg ctccctagct gtgggtcaga actggttatt ggatgagcaa
  481 accagcaccc tcacaaggct gcgctactct taccgggtca tctgcagtga caactactat
  541 ggagacaact gctcccgcct gtgcaagaag cgcaatgacc acttcggcca ctatgtgtgc
  601 cagccagatg gcaacttgtc ctgcctgccc ggttggactg gggaatattg ccaacagcct
  661 atctgtcttt cgggctgtca tgaacagaat ggctactgca gcaagccagc agagtgcctc
  721 tgccgcccag gctggcaggg ccggctgtgt aacgaatgca tcccccacaa tggctgtcgc
  781 cacggcacct gcagcactcc ctggcaatgt acttgtgatg agggctgggg aggcctgttt
  841 tgtgaccaag atctcaacta ctgcacccac cactccccat gcaagaatgg ggcaacgtgc
  901 tccaacagtg ggcagcgaag ctacacctgc acctgtcgcc caggctacac tggtgtggac
  961 tgtgagctgg agctcagcga gtgtgacagc aacccctgtc gcaatggagg cagctgtaag
1021 gaccaggagg atggctacca ctgcctgtgt cctccgggct actatggcct gcattgtgaa
1081 cacagcacct tgagctgcgc cgactccccc tgcttcaatg ggggctcctg ccgggagcgc
1141 aaccaggggg ccaactatgc ttgtgaatgt ccccccaact tcaccggctc caactgcgag
1201 aagaaagtgg acaggtgcac cagcaacccc tgtgccaacg ggggacagtg cctgaaccga
1261 ggtccaagcc gcatgtgccg ctgccgtcct ggattcacgg gcacctactg tgaactccac
1321 gtcagcgact gtgcccgtaa cccttgcgcc cacggtggca cttgccatga cctggagaat
1381 gggctcatgt gcacctgccc tgccggcttc tctggccgac gctgtgaggt gcggacatcc
1441 atcgatgcct gtgcctcgag tccctgcttc aacagggcca cctgctacac cgacctctcc
1501 acagacacct ttgtgtgcaa ctgcccttat ggctttgtgg gcagccgctg cgagttcccc
1561 gtgggcttgc cgcccagctt cccctgggtg gccgtctcgc tgggtgtggg gctggcagtg
1621 ctgctggtac tgctgggcat ggtggcagtg gctgtgcggc agctgcggct tcgacggccg
1681 gacgacggca gcagggaagc catgaacaac ttgtcggact tccagaagga caacctgatt
1741 cctgccgccc agcttaaaaa cacaaaccag aagaaggagc tggaagtgga ctgtggcctg
1801 gacaagtcca actgtggcaa acagcaaaac cacacattgg actataatct ggccccaggg
1861 cccctggggc gggggaccat gccaggaaag tttccccaca gtgacaagag cttaggagag
1921 aaggcgccac tgcggttaca cagtgaaaag ccagagtgtc ggatatcagc gatatgctcc
1981 cccagggact ccattacca gtctgtgtgt ttgatatcag aggagaggaa tgaatgtgtc
2041 attgccacgg aggtataa
Delta-like ligand 4 amino acid sequence (AF253468.1)
SEQ ID NO: 8
MAAASRSASGWALLLLVALWQQRAAGSGVFQLQLQEFINERGVLASGRPCEPGCRTFFRVCLKHFQAVVSPGPCT
FGTVSTPVLGTNSFAVRDDSSGGGRNPLQLPFNFTWPGTESLIIEAWHAPGDDLRPEALPPDALISKIAIQGSLA
VGQNWLLDEQTSTLTRLRYSYRVICSDNYYGDNCSRLCKKRNDHFGHYVCQPDGNLSCLPGWTGEYCQQPICLSG
CHEQNGYCSKPAECLCRPGWQGRLCNECIPHNGCRHGTCSTPWQCTCDEGWGGLFCDQDLNYCTHHSPCKNGATC
SNSGQRSYTCTCRPGYTGVDCELELSECDSNPCRNGGSCKDQEDGYHCLCPPGYYGLHCEHSTLSCADSPCENGG
SCRERNQGANYACECPPNFTGSNCEKKVDRCTSNPCANGGQCLNRGPSRMCRCRPGFTGTYCELHVSDCARNPCA
HGGTCHDLENGLMCTCPAGESGRRCEVRTSIDACASSPCFNRATCYTDLSTDTFVCNCPYGFVGSRCEFPVGLPP
SFPWVAVSLGVGLAVLLVLLGMVAVAVRQLRLRRPDDGSREAMNNLSDFQKDNLIPAAQLKNTNQKKELEVDCGL
DKSNCGKQQNHTLDYNLAPGPLGRGTMPGKFPHSDKSLGEKAPLRLHSEKPECRISAICSPRDSMYQSVCLISEE
RNECVIATEV
Human Notch1 cDNA sequence (CR457221.1)
SEQ ID NO: 9
      1 atgtcaaaca tgagatgtgt ggactgtggc acttgcctgg gtcacacacg gaggcatcct
    61 acccttttct ggggaaagac actgcctggg ctgaccccgg ggcggcccc agcacctcag
  121 cctgcacagt gtcccccagg ttccgaagaa gatgctccag caacacagcc tgggccccag
  181 ctcgcgggac ccgacccccc gtgggctccc gtgttttgta ggagacttgc cagagccggg
  241 cacattgagc tgtgcaacgc cgtgggctgc gtcctttggt cctgtccccg cagccctggc
  301 agggggcatg cggtcgggca ggggctggag ggaggcgggg gctgcccttg ggccacccct
  361 cctagtttgg gaggagcaga tttttgcaat accaagtata gcctatggca gaaaaaatgt
  421 ctttaa
Human Notch1 protein sequence (CR457221.1)
SEQ ID NO: 10
MSNMRCVDCGTCLGHTRRHPTLFWGKTLPGLTPVAAPAPQPAQCPPGSEEDAPATQPGPQLAGPDPPWAPVFCRR
LARAGHIELCNAVGCVLWSCPRSPGRGHAVGQGLEGGGGCPWATPPSLGGADFCNTKYSLWQKKCL
```

### EXAMPLES

The invention is now described with reference to the Examples below. These are not limiting on the scope of the invention, and a person skilled in the art would be appreciate that suitable equivalents could be used within the scope of the present invention. Thus, the Examples may be considered component parts of the invention, and the individual aspects described therein may be considered as disclosed independently, or in any combination.

### Example 1—Inclusion of a Pre-Differentiation Step Increases CD16⁺ NK Cell Production

CD34⁺ HPCs were isolated from human umbilical cord blood and frozen prior to testing. Isolated CD34⁺ HPCs were thawed, allowed to recover overnight. The HPC were then cultured in a non-differentiation medium for a period of 0, 2, 4 or 6 days. Following this pre-differentiation step, the pre-differentiation HPC population was transferred to a fresh culture vessel and cultured in a differentiation medium for a further 20 days. At the end of this differentiation period, the cells were analysed by flow cytometry to quantify CD56 and CD16 expression.

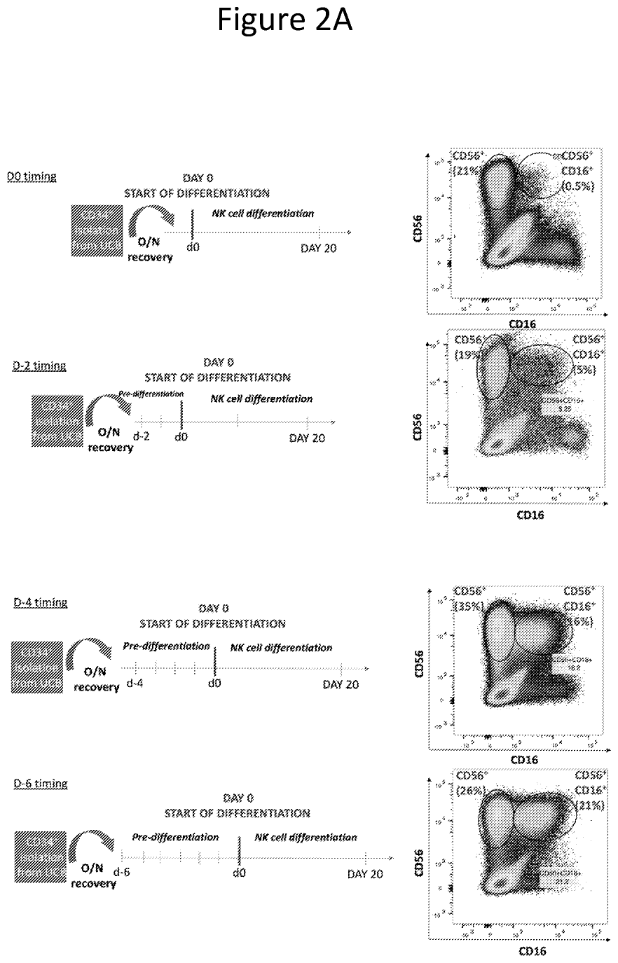

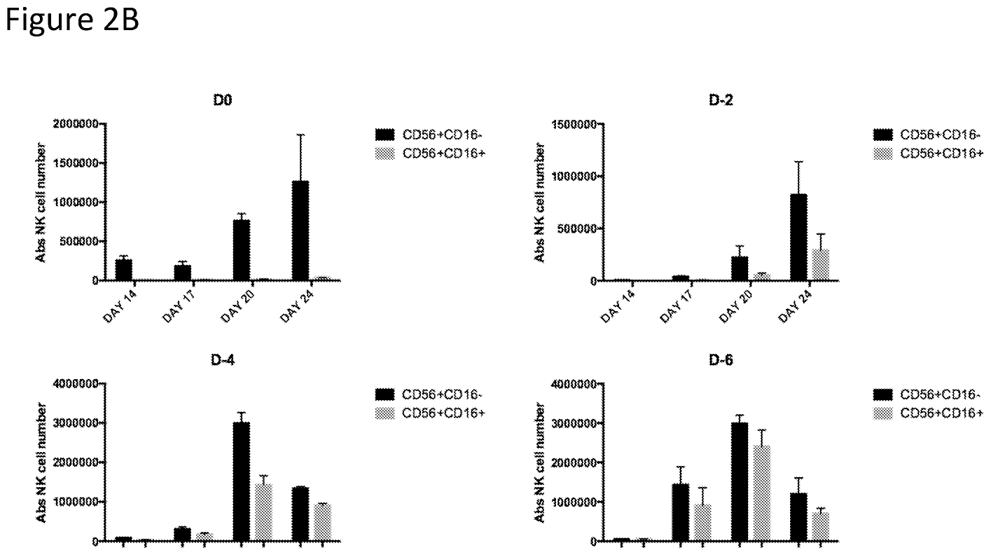

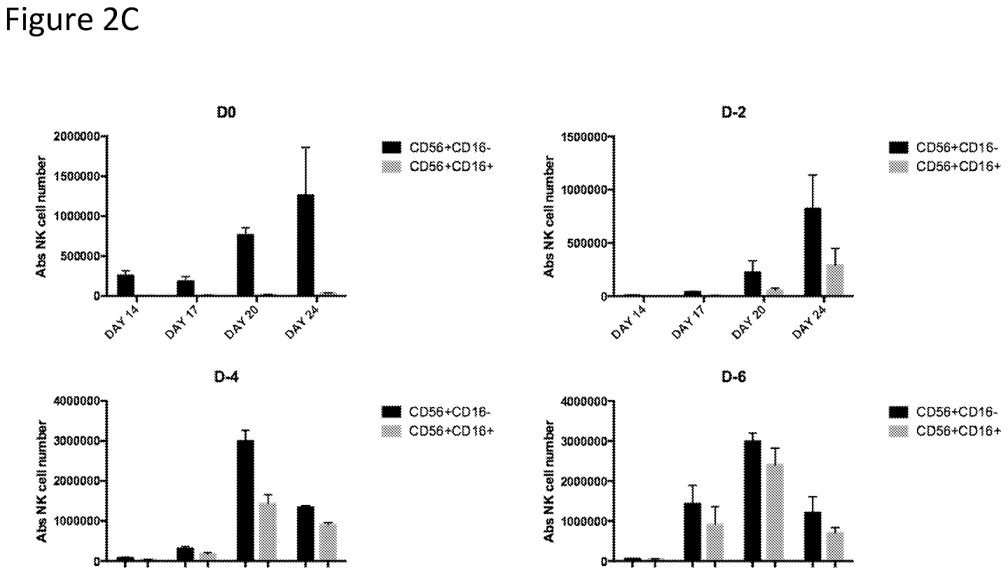

As shown in FIG. 2A, omitting the pre-differentiation step resulted in a final NK cell population with moderate CD56 expression (CD56⁺ of 21%), but low CD16 expression (CD16⁺ of 0.5%). As the length of time of the pre-differentiation culture period increased, there was a significant increase in the % of CD16⁺ NK cells: d2 (CD56⁺ of 35%, CD16⁺ of 16%), d6 (CD56⁺ of 26%, CD16⁺ of 21%). Extending the pre-differentiation culture period beyond 6 days results in a reduction in NK cell production (data not shown), with NK progenitor potential diminished beyond 6 days in pre-differentiation culture.

These flow cytometry data is quantified in FIGS. 2B and 2C. FIG. 2B shows the absolute number of human NK cells generated after 20 days differentiation on stromal cells. DO, D-2, D-4 & D-6 refers to the time the cells spent in pre-differentiation before being placed on stromal cells (e.g. D-6=6 days pre-differentiation before transfer to stromal cells for differentiation). The NK cells are displayed as either CD56+CD16− (NK cells not expressing CD16) or CD56+CD16+(NK cells expressing CD16+). Total NK cells includes both CD56+CD16− and CD56+CD16+ cells. FIG. 2B shows that there is an increase in CD16+ NK cells when a pre-differentiation step culture step is included, compared with D=), wherein the CD34+ HPCs are taken straight from post-thaw recovery to differentiation, with the biggest increase in CD16+ NK cells seen for D=6.

FIG. 2C shows the same experimental data, with the numbers of cells produced at different lengths of time differentiating on stromal cells plotted against the number of days of pre-differentiation. The highest number of CD56+CD16+ NK cells are found after 6 days pre-differentiation and 20 days subsequent differentiation on stroma, with significant numbers of CD56+CD16+ NK cells also observed after 6 days pre-differentiation and 20 days subsequent differentiation on stroma.

Thus, surprisingly, these data show that with no exogenous factors added, it is possible to elicit a significant increase in CD16 expression merely by defining a set pre-differentiation culture period.

### Example 2—Inclusion of DLL4 Further Increases CD16⁺ NK Cell Production

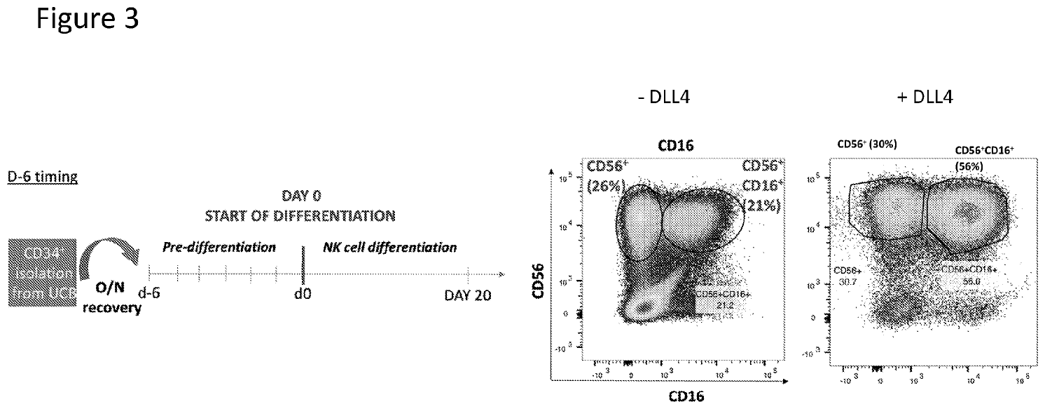

The experiment in Example 1 was repeated using a recovery period of 2 days and pre-differentiation period of 6 days, wherein the HPCs were cultured in the presence or absence of DLL4 peptide (coated on the plate at a concentration of 2 μg/ml). The presence of DLL4 peptide was via a DLL4-coated plate. As shown in FIG. 3, inclusion of DLL4 further increased CD16⁺ expression, with a significant increase to 56% of the resulting NK cells being CD16⁺.

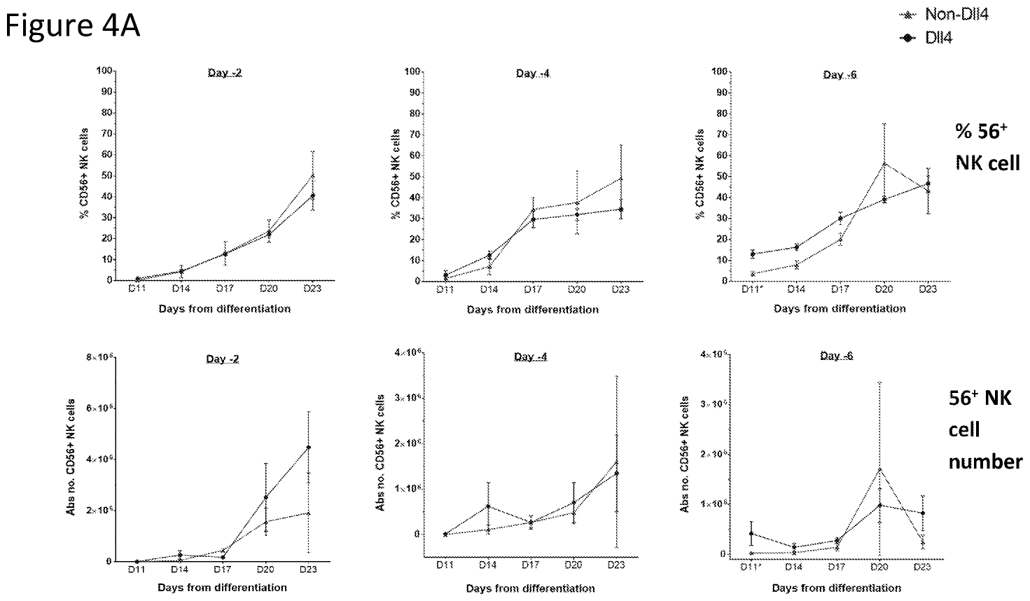

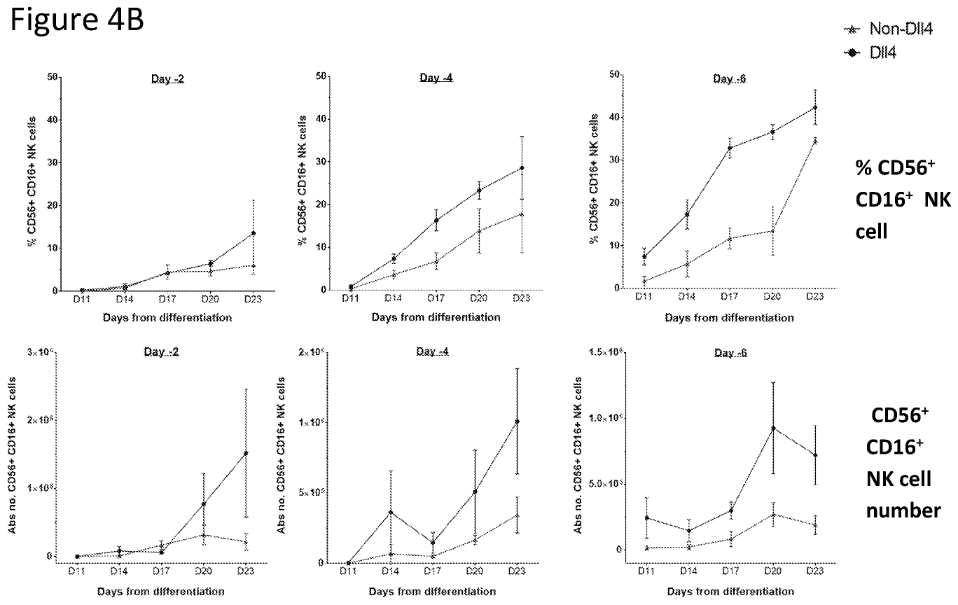

Further replicates in which the presence/absence of DLL4 with pre-differentiation periods of 2, 4 and 6 days were conducted. FIG. 4A shows the results expressed as a time course and shows the percentage (upper panels) and absolute number (lower panels) of CD56⁺ cells. FIG. 4B shows the results expressed as a time course and shows the percentage (upper panels) and absolute numbers (lower panels) of CD56⁺CD16⁺ cells either with (●) DLL4 or without (▴) DLL4. These results demonstrate that, whilst there was no significant effect on the total number of NK cells (CD56⁺, FIG. 4A), the number of CD16⁺ NK cells further increased when DLL4 was combined with the pre-differentiation step (FIG. 4B).

### Example 3—Omission of IL-3 Further Increases CD16⁺ NK Cell Production when Cells are Cultured with DLL4

CD34⁺ HPCs from human umbilical cord blood were treated with either DLL4 or with vehicle control only (CTRL) for 4 days prior to transfer to stromal cells (+EL08). Controls without EL08 (−EL08) were also tested. IL-3 was added (+IL-3) or withheld (−IL-3) and the percentage of CD56⁺CD16⁺ NK cells produced was measured over time of differentiation.

As shown in FIG. 5, treatment with DLL4 results in approximately 25% more CD56⁺CD16⁺ cells by day 30 compared to the respective control. Removal of IL-3 further increases the % of CD56⁺CD16⁺ cells in the absence of stromal cells or when cultured on DLL4 but not in the normal control (on stromal cells).

### Example 4—Increasing the Duration of the Pre-Differentiation Step Reduces CD16⁺ NK Cell Production

CD34⁺ HPCs were isolated from human umbilical cord blood and frozen prior to testing. Isolated CD34⁺ HPCs were thawed, allowed to recover overnight. The HPC were then divided into two populations, each cultured in a non-differentiation medium for a period of either 4 or 14 days. Following this pre-differentiation step, each pre-differentiation HPC population was transferred to a fresh culture vessel and cultured in a differentiation medium for a further 23 days. At the end of this differentiation period, the cells were analysed by flow cytometry to quantify CD56 and CD16 expression.

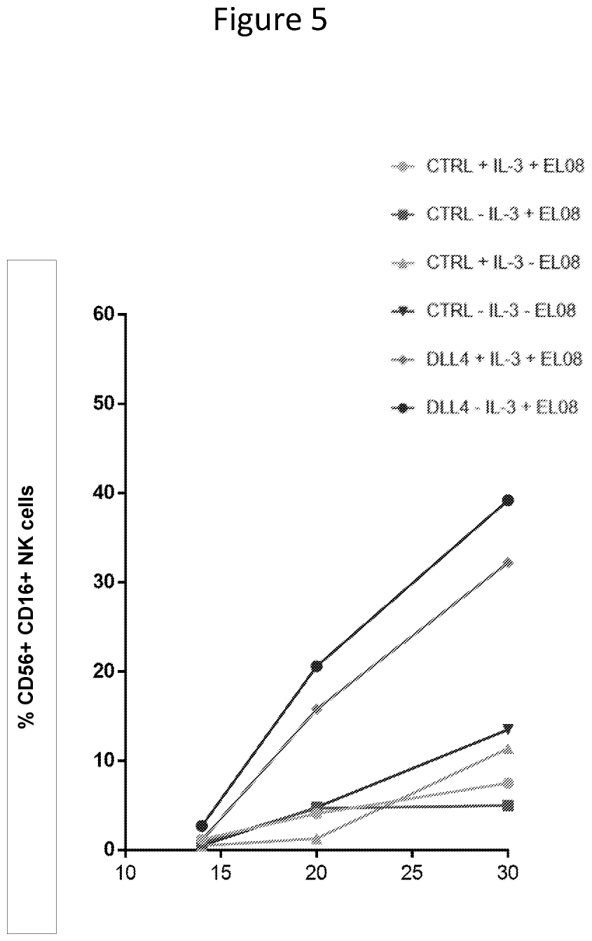

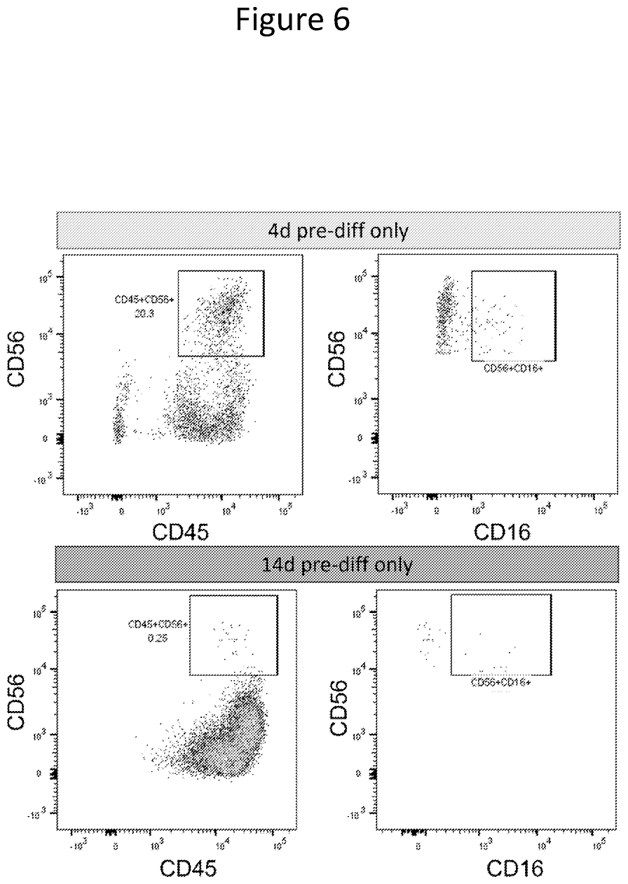

As shown in FIG. 6, increasing the duration of the pre-differentiation culture step to 14 days significantly reduced the number of CD56⁺CD45⁺ NK cells produced (top left panel=pre-differentiation for 4 days, bottom left=pre-differentiation for 14 days), with 20.3% of the cells produced using a 4 day pre-incubation being CD56⁺CD45⁺ NK cells compared with 0.25% CD56⁺CD45⁺ NK cells when the pre-differentiation period was 14 days.

Furthermore, the number of CD16⁺ NK cells was effectively reduced to zero in the cell population cultured using a 14-day pre-incubation period (top right panel=pre-differentiation for 4 days, bottom right=pre-differentiation for 14 days), whereas a significant minority of the resulting CD56⁺CD45⁺ NK cells produced using the 4-day pre-differentiation period were also CD16⁺.

These results suggest that increasing the duration of the pre-differentiation period beyond an upper threshold, particularly to 14 days have a significant negative effect on the production of mature (i.e. CD56⁺CD45⁺CD16⁺) NK cells.

### Example 5—Inclusion of DLL4 and/or a REV-ERB Inhibitor Further Increases NK Cell Production

The effect of the Notch ligand DLL4 and/or a REV-ERB inhibitor were tested in combination with a 4-day pre-differentiation period.

Following the pre-differentiation step, each pre-differentiation HPC population was transferred to a fresh culture vessel and cultured in a differentiation medium for a further 23 days. At the end of this differentiation period, the cells were analysed by flow cytometry to quantify CD56 and CD45 expression.

When DLL4 was used, this was used (at a concentration of 2 μg/ml) to coat the culture plate and was present from d−4 (i.e. 4 days before the change from pre-differentiation medium to differentiation medium). The DLL4 was not present in the differentiation medium.

When the REV-ERB inhibitor was present, this used at a final concentration of 2 μM and was added on d−5 (i.e. 5 days before the change from pre-differentiation medium to differentiation medium). REV-ERB inhibitor was not included differentiation medium.

When REV-ERB inhibitor (2 μM) and DLL4 (used to coat plates at a concentration of 2 μg/ml), the REV-ERB inhibitor was present from day −5 and the DLL4 was present from day −4. For the differentiation step, REV-ERB inhibitor was not included differentiation medium and the cells were plated on EL08-ID2 cells.

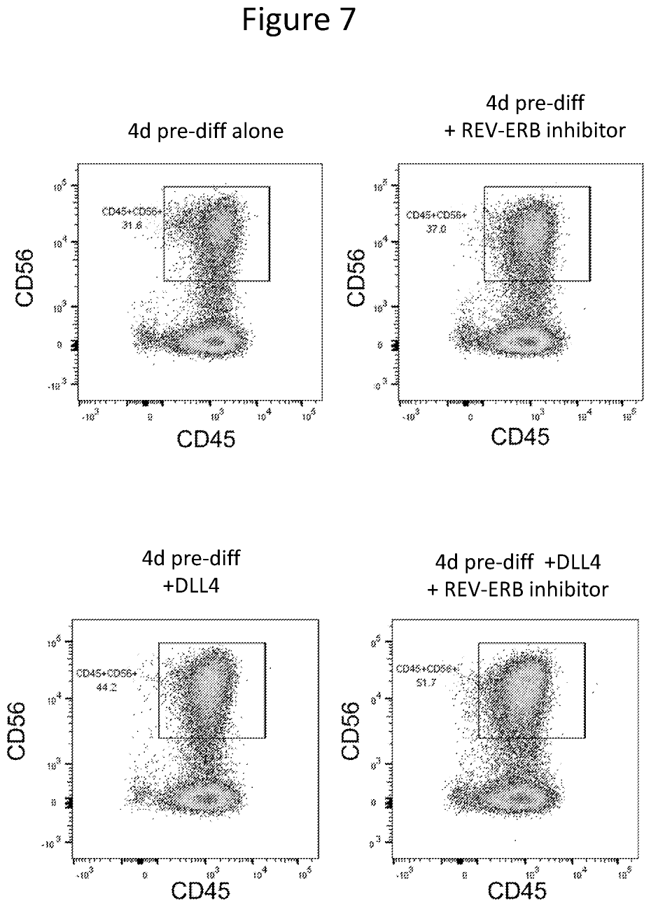

As shown in FIG. 7, the 4-day pre-differentiation period alone resulted in 31.6% of the total cells being CD45⁺CD56⁺. The addition of a REV-ERB inhibitor further increased the proportion of CD45⁺CD56⁺ cells to 37%. Combining DLL4 with the 4-day pre-differentiation period resulted in 44.2% of the cells being CD45⁺CD56⁺. The combination of a 4-day pre-differentiation with both DLL4 and REV-ERB inhibition had the greatest effect on NK cell numbers, with 51.7% of the resulting cells being CD45⁺CD56⁺.

Thus the presence of DLL4; the REV-ERB inhibitor; or DLL4 and the REV-ERB inhibitor increased the number of CD45⁺CD56⁺ NK cells produced with the following trend: pre-differentiation<pre-differentiation+REV-ERB inhibitor<pre-differentiation+DLL4<pre-differentiation+DLL4+REV-ERB inhibitor. Furthermore, this same trend was reflected in terms of the number of CD16 expression. Thus, the number of CD45⁺CD56⁺CD16⁺ NK cells produced also followed the trend: pre-differentiation<pre-differentiation+REV-ERB inhibitor<<pre-differentiation+DLL4<<pre-differentiation+DLL4+REV-ERB inhibitor (data not shown).

Therefore, these data demonstrate that methods of the invention, particularly those in which a pre-differentiation step is combined with DLL4 and/or a REV-ERB inhibitor, and preferably both DLL4 and a REV-ERB inhibitor can not only increase the number of NK cells produced, but also increase the number of CD16⁺ NK cells.

### Example 6—Comparison of KIR Expression of CD16⁺ NK Cells and CD16⁻ NK Cells Produced by a Method Comprising a Pre-Differentiation Step

CD34⁺ HPCs were isolated from human umbilical cord blood and frozen prior to testing. Isolated CD34⁺ HPCs were thawed, allowed to recover overnight. The HPC were then cultured in a non-differentiation medium for a period of 6 days. Following this pre-differentiation step, the pre-differentiation HPC population was transferred to a fresh culture vessel and cultured in a differentiation medium for a further 23 days. At the end of this differentiation period, the cells were analysed by flow cytometry to quantify CD56 and CD16 expression. The resulting CD56⁺CD16⁺ NK cells and CD56⁺CD16⁻ NK cells were further analysed for KIR expression.

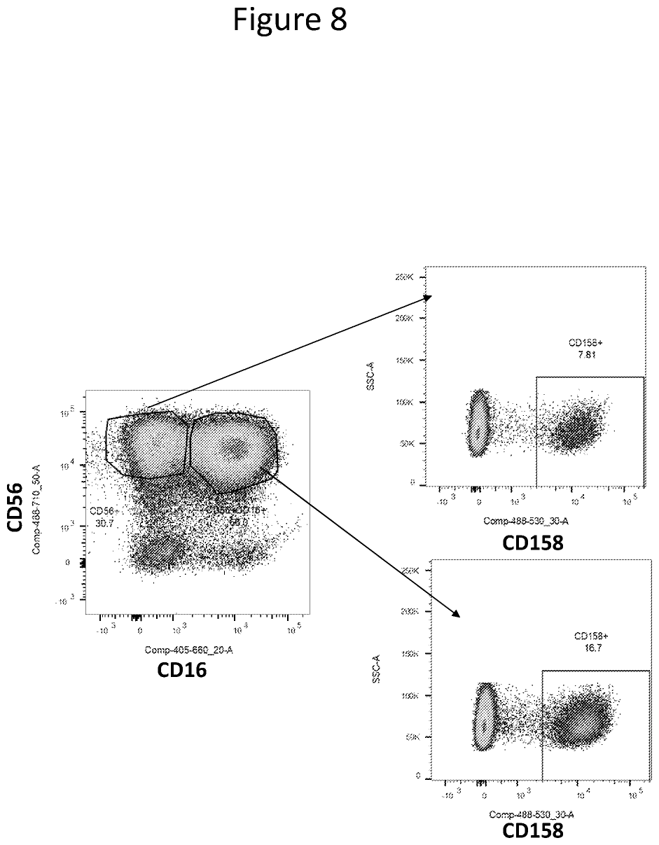

As shown in FIG. 8, both the CD56⁺CD16⁺ NK cells and CD56⁺CD16⁻ NK cells were positively detected using an antibody for CD158. This anti CD158 antibody is cross-reactive for KIR2DL1, KIR2DS1, KIR2DS3 and KIR2DS5. Therefore, the CD56⁺CD16⁺ NK cells and CD56⁺CD16⁻ NK cells express one or more of these KIR molecules. Furthermore, the CD56⁺CD16⁺ NK cells were enriched for expression of these KIR proteins compared with the CD56⁺CD16⁻ NK cells portion.

1. An ex vivo method for producing an expanded population of CD16⁺ Natural Killer (NK) cells, comprising the steps of:
a) culturing an haematopoietic progenitor cell (HPC) comprising sample obtained from an individual in medium which does not induce differentiation of the HPCs for between about 2 to about 8 days to produce a pre-differentiation HPC population; and
b) culturing the pre-differentiation HPC population in medium which induces differentiation of the HPCs to NK.

2. An ex vivo method for increasing the number of CD16⁺ NK cells in an expanded NK cell population, comprising the steps of:
a) culturing an haematopoietic progenitor cell (HPC) comprising sample obtained from an individual in medium which does not induce differentiation of the HPCs for between about 2 to about 8 days to produce a pre-differentiation HPC population; and
b) culturing the pre-differentiation HPC population in medium which induces differentiation of the HPCs to NK.

3. A method according to claim 1 or 2, wherein in step (a) the HPCs are cultured for between about 2 days to about 6 days, optionally for between about 4 days to about 6 days.

4. A method according to any one of the preceding claims, wherein the HPCs are cultured in the presence of a Notch ligand for at least part of step (a).

5. A method according to claim 4, wherein:
(i) the HPCs are cultured in a vessel that is coated with the Notch ligand for at least part of step (a); and/or
(ii) the Notch ligand is delta-like ligand 4 (DLL4), or a fragment thereof which retains the function of DLL4;
wherein preferably in step (a) the HPCs are cultured in the absence of the Notch ligand for about 1 day followed by culture in the presence of the Notch ligand for the remainder of step (a).

6. A method according to any one of the preceding claims, wherein:
(i) in step (a) the HPCs are cultured in the presence of a compound which inhibits the action of REV-ERB; and/or
(ii) in step (b) the pre-differentiation HPC population is cultured in the presence of a compound which inhibits the action of REV-ERB.

7. A method according to claim 6, wherein said compound:
(i) increases E4bp4 expression by decreasing REV-ERB activity;
(ii) decreases the activity of REV-ERB-α and/or REV-ERB-β, preferably REV-ERB-β;
(iii) decreases the activity of REV-ERB-α and REV-ERB-β;
(iv) is a REV-ERB antagonist, preferably an antagonist of REV-ERB-α and REV-ERB-β;
(v) is selected from a small molecule, a PROTAC reagent, a double stranded RNA (dsRNA), a small interfering RNA (siRNA), a small hairpin RNA (shRNA), a micro RNA, an antisense RNA, an aptamer, an antibody, a ribozyme, a peptide or a peptidomimetic, preferably a small molecule; and/or
(vi) is SR8278, 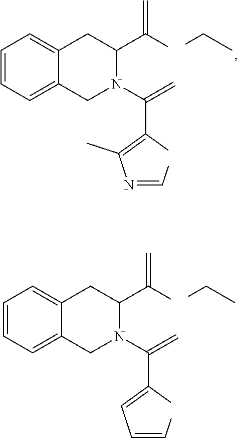 [SMILES: C=C(CCC)C1Cc2ccccc2CN1C(=C)C1=C(C)N=CC1 ; C=C(CCC)C1Cc2ccccc2CN1C(=C)C1=CC=CC1]
or GSK1362.

8. A method according to any one of the preceding claims, wherein the medium which does not induces differentiation of the HPCs in step (a) and/or the medium which induces differentiation of the HPCs to NK in step (b) does not comprise IL-3, preferably wherein the medium which induces differentiation of the HPCs to NK in step (b) does not comprise IL-3.

9. A method according to any one of the preceding claims, wherein:
(i) the medium in step (a) comprises at least one of Flt3L, GM-CSF, IL-3, IL-6, TPO and/or stem cell factor (SCF), preferably Flt3L, GM-CSF, IL-3, IL-6, TPO and SCF; and/or
(ii) the medium in step (b) comprises IL-7, Flt3L, IL-15, and/or SCF, preferably IL-7, Flt3L, IL-15 and SCF.

10. A method according to any one of the preceding claims, wherein the step (a) and/or step (b) is carried out in the absence of a stromal support cell, preferably wherein both step (a) and step (b) are carried out in the absence of a stromal support cell.

11. A method according to any one of the preceding claims, wherein the sample of HPCs is obtained from bone marrow, cord blood and/or peripheral blood.

12. A method according to any one of the preceding claims, wherein the proportion of CD16⁺ NK cells is increased compared with the proportion of CD16⁺ NK cells produced by a corresponding method in which step (a) is omitted.

13. A method according to any one of the preceding claims, wherein the expanded NK cell population comprises at least 10% CD16⁺ NK cells, preferably at least 15% CD16⁺ NK cells, more preferably at least 20% CD16⁺ NK cells, even more preferably at least 30% CD16⁺ NK cells.

14. A method according to any one of the preceding claims, wherein the expanded NK cell population exhibits at least 30% greater antibody-dependent cellular cytotoxicity (ADCC), preferably at least 50% greater ADCC, compared with NK cells produced by a corresponding method in which step (a) is omitted.

15. A method according to any one of the preceding claims, wherein which does not comprise a further step of introducing exogenous nucleic acid into the HPCs and/or NK cells.

16. An expanded population of CD16⁺ NK cells, wherein at least 10% of the NK cells are CD16⁺ NK cells, preferably at least 15% of the NK cells are CD16⁺ NK cells, more preferably at least 20% of the NK cells are CD16⁺ NK cells, even more preferably at least 30% of the NK cells are CD16⁺ NK cells.

17. An expanded population of CD16⁺ NK cells obtained by the method of any one of claims 1 to 15, wherein at least 10% of the NK cells are CD16⁺ NK cells, preferably at least 15% of the NK cells are CD16⁺ NK cells, more preferably at least 20% of the NK cells are CD16⁺ NK cells, even more preferably at least 30% of the NK cells are CD16⁺ NK cells.

18. An expanded population of CD16⁺ NK cells according to claim 16 or 17, wherein the CD16⁺ NK cells do not comprise exogenous nucleic acid.

19. An expanded population of CD16⁺ NK cells according to any one of claims 16 to 18, wherein the expanded NK cell population exhibits at least 30% greater ADCC, preferably at least 50% greater ADCC, more preferably at least 70% greater ADCC, compared with NK cells produced by a corresponding method in which step (a) is omitted.

20. A composition comprising an expanded NK cell population as defined in any one of claims 16 to 19 and a pharmaceutically acceptable carrier, diluent and/or excipient.

21. An expanded population of CD16⁺ NK cells as defined in any one of claims 16 to 19 or a composition as defined in claim 20 for use in a method of therapy.

22. An expanded population of CD16⁺ NK cells or composition for use according to claim 21, wherein said method of therapy is a method of treating a disease or disorder selected from cancer, an infectious disease (acute or chronic), an autoimmune disease or a disease or disorder related to female infertility or pregnancy.

23. An expanded population of CD16⁺ NK cells or composition for use according to claim 21 or 22, wherein said method of therapy is a method of treatment of a viral infection, a bacterial infection, a protist infection, a fungal infection and/or a helminth infection.

24. An expanded population of CD16⁺ NK cells or composition for use according to any one of claims 21 to 23, which is used in combination with antibody-mediated immunotherapy.

25. An expanded population of CD16⁺ NK cells or composition for use according to claim 24, wherein said expanded population of CD16⁺ NK cells or composition is for administration before, simultaneously with, or after administration of the antibody-mediated immunotherapy.


SEQUENCE LISTING (10 sequences)

SEQ ID NO: 1 - RNA, 1923 nt, Homo sapiens
  source 1..1923: mol_type = mRNA
gcccctttctttctcctcgtcggcccgagagcaggaacacgataacgaaggaggcccaacttcattcaataaggagcctgacggatttatcccagacggtagaacaaaaggaagaatattgatggattttaaaccagagtttttaaagagcttgagaatacggggaaattaatttgttctcctacacacatagatagggtaaggttgtttctgatgcagctgagaaaaatgcagaccgtcaaaaaggagcaggcgtctcttgatgccagtagcaatgtggacaagatgatggtccttaattctgctttaacggaagtgtcagaagactccacaacaggtgaggacgtgcttctcagtgaaggaagtgtggggaagaacaaatcttctgcatgtcggaggaaacgggaattcattcctgatgaaaagaaagatgctatgtattgggaaaaaaggcggaaaaataatgaagctgccaaaagatctcgtgagaagcgtcgactgaatgacctggttttagagaacaaactaattgcactgggagaagaaaacgccactttaaaagctgagctgctttcactaaaattaaagtttggtttaattagctccacagcatatgctcaagagattcagaaactcagtaattctacagctgtgtactttcaagattaccagacttccaaatccaatgtgagttcatttgtggacgagcacgaaccctcgatggtgtcaagtagttgtatttctgtcattaaacactctccacaaagctcgctgtccgatgtttcagaagtgtcctcagtagaacacacgcaggagagctctgtgcagggaagctgcagaagtcctgaaaacaagttccagattatcaagcaagagccgatggaattagagagctacacaagggagccaagagatgaccgaggctcttacacagcgtccatctatcaaaactatatggggaattctttctctgggtactcacactctcccccactactgcaagtcaaccgatcctccagcaactccccgagaacgtcggaaactgatgatggtgtggtaggaaagtcatctgatggagaagacgagcaacaggtccccaagggccccatccattctccagttgaactcaagcatgtgcatgcaactgtggttaaagttccagaagtgaattcctctgccttgccacacaagctccggatcaaagccaaagccatgcagatcaaagtagaagcctttgataatgaatttgaggccacgcaaaaactttcctcacctattgacatgacatctaaaagacatttcgaactcgaaaagcatagtgccccaagtatggtacattcttctcttactcctttctcagtgcaagtgactaacattcaagattggtctctcaaatcggagcactggcatcaaaaagaactgagtggcaaaactcagaatagtttcaaaactggagttgttgaaatgaaagacagtggctacaaagtttctgacccagagaacttgtatttgaagcaggggatagcaaacttatctgcagaggttgtctcactcaagagacttatagccacacaaccaatctctgcttcagactctgggtaaattactactgagtaagagctgggcatttagaaagatgtcatttgcaatagagcagtccattttgtattatgctgaattttcactggacctgtgatgtcatttcactgtgatgtgcacatgttgtctgtttggtgtctttttgtgcacagattatgatgaagattagattgtgttatcactctgcctgtgtatagtcagatagtcatatgcgtaaggctgtatatattaagnttttatttttgttgttctattataaagtgtgtaagttaccagtttcaataaaggattggtgacaaacacagaaaaaaaaaaaaaaaaaaa

SEQ ID NO: 2 - protein, 462 aa, Homo sapiens [sequence omitted]

SEQ ID NO: 3 - RNA, 2793 nt, Homo sapiens
  source 1..2793: mol_type = mRNA
gggcacgaggcgctccctgggatcacatggtacctgctccagtgccgcgtgcggcccgggaaccctgggctgctggcgcctgcgcagagccctctgtcccagggaaaggctcgggcaaaaggcggctgagattggcagagtgaaatattactgccgagggaacgtagcagggcacacgtctcgcctctttgcgactcggtgccccgtttctccccatcacctacttacttcctggttgcaacctctcttcctctgggacttttgcaccgggagctccagattcgccaccccgcagcgctgcggagccggcaggcagaggcaccccgtacactgcagagacccgaccctccttgctaccttctagccagaactactgcaggctgattccccctacacactctctctgctcttcccatgcaaagcagaactccgttgcctcaacgtccaacccttctgcagggctgcagtccggccaccccaagaccttgctgcagggtgcttcggatcctgatcgtgagtcgcggggtccactccccgcccttagccagtgcccagggggcaacagcggcgatcgcaacctctagtttgagtcaaggtccagtttgaatgaccgctctcagctggtgaagacatgacgaccctggactccaacaacaacacaggtggcgtcatcacctacattggctccagtggctcctccccaagccgcaccagccctgaatccctctatagtgacaactccaatggcagcttccagtccctgacccaaggctgtcccacctacttcccaccatcccccactggctccctcacccaagacccggctcgctcctttgggagcattccacccagcctgagtgatgacggctccccttcttcctcatcttcctcgtcgtcatcctcctcctccttctataatgggagcccccctgggagtctacaagtggccatggaggacagcagccgagtgtcccccagcaagagcaccagcaacatcaccaagctgaatggcatggtgttactgtgtaaagtgtgtggggacgttgcctcgggcttccactacggtgtgcacgcctgcgagggctgcaagggctttttccgtcggagcatccagcagaacatccagtacaaaaggtgtctgaagaatgagaattgctccatcgtccgcatcaatcgcaaccgctgccagcaatgtcgcttcaagaagtgtctctctgtgggcatgtctcgagacgctgtgcgttttgggcgcatccccaaacgagagaagcagcggatgcttgctgagatgcagagtgccatgaacctggccaacaaccagttgagcagccagtgcccgctggagacttcacccacccagcaccccaccccaggccccatgggcccctcgccaccccctgctccggtcccctcacccctggtgggcttctcccagtttccacaacagctgacgcctcccagatccccaagccctgagcccacagtggaggatgtgatatcccaggtggcccgggcccatcgagagatcttcacctacgcccatgacaagctgggcagctcacctggcaacttcaatgccaaccatgcatcaggtagccctccagccaccaccccacatcgctgggaaaatcagggctgcccacctgcccccaatgacaacaacaccttggctgcccagcgtcataacgaggccctaaatggtctgcgccaggctccctcctcctaccctcccacctggcctcctggccctgcacaccacagctgccaccagtccaacagcaacgggcaccgtctatgccccacccacgtgtatgcagccccagaaggcaaggcacctgccaacagtccccggcagggcaactcaaagaatgttctgctggcatgtcctatgaacatgtacccgcatggacgcagtgggcgaacggtgcaggagatctgggaggatttctccatgagcttcacgcccgctgtgcgggaggtggtagagtttgccaaacacatcccgggcttccgtgacctttctcagcatgaccaagtcaccctgcttaaggctggcacctttgaggtgctgatggtgcgctttgcttcgttgttcaacgtgaaggaccagacagtgatgttcctaagccgcaccacctacagcctgcaggagcttggtgccatgggcatgggagacctgctcagtgccatgttcgacttcagcgagaagctcaactccctggcgcttaccgaggaggagctgggcctcttcaccgcggtggtgcttgtctctgcagaccgctcgggcatggagaattccgcttcggtggagcagctccaggagacgctgctgcgggctcttcgggctctggtgctgaagaaccggcccttggagacttcccgcttcaccaagctgctgctcaagctgccggacctgcggaccctgaacaacatgcattccgagaagctgctgtccttccgggtggacgcccagtgacccgcccggccggccttctgccgctgcccccttgtacagaatcgaactctgcacttctctctcctttacgagacgaaaaggaaaagcaaaccagaatcttatttatattgttataaaatattccaagatgagcctctggccccctgagccttcttgtaaatacctgcctccctcccccatcaccgaacttcccctcctcccctatttaaaccactctgtctcccccacaaccctcccctggccctctgatttgttctgttcctgtctcaaatccaatagttcacagctgagctggcttcaaaaaaaaaaaaaaaaaa

SEQ ID NO: 4 - protein, 614 aa, Homo sapiens
MTTLDSNNNTGGVITYIGSSGSSPSRTSPESLYSDNSNGSFQSLTQGCPTYFPPSPTGSLTQDPARSFGSIPPSLSDDGSPSSSSSSSSSSSSFYNGSPPGSLQVAMEDSSRVSPSKSTSNITKLNGMVLLCKVCGDVASGFHYGVHACEGCKGFFRRSIQQNIQYKRCLKNENCSIVRINRNRCQQCRFKKCLSVGMSRDAVRFGRIPKREKQRMLAEMQSAMNLANNQLSSQCPLETSPTQHPTPGPMGPSPPPAPVPSPLVGFSQFPQQLTPPRSPSPEPTVEDVISQVARAHREIFTYAHDKLGSSPGNFNANHASGSPPATTPHRWENQGCPPAPNDNNTLAAQRHNEALNGLRQAPSSYPPTWPPGPAHHSCHQSNSNGHRLCPTHVYAAPEGKAPANSPRQGNSKNVLLACPMNMYPHGRSGRTVQEIWEDFSMSFTPAVREVVEFAKHIPGFRDLSQHDQVTLLKAGTFEVLMVRFASLFNVKDQTVMFLSRTTYSLQELGAMGMGDLLSAMFDFSEKLNSLALTEEELGLFTAVVLVSADRSGMENSASVEQLQETLLRALRALVLKNRPLETSRFTKLLLKLPDLRTLNNMHSEKLLSFRVDAQ

SEQ ID NO: 5 - RNA, 1740 nt, Homo sapiens
  source 1..1740: mol_type = mRNA
atggaggtgaatgcaggaggtgtgattgcctatatcagttcttccagctcagcctcaagccctgcctcttgtcacagtgagggttctgagaatagtttccagtcctcctcctcttctgttccatcttctccaaatagctctaattctgataccaatggtaatcccaagaatggtgatctcgccaatattgaaggcatcttgaagaatgatcgaatagattgttctatgaaaacaagcaaatcgagtgcacctgggatgacaaaaaatcatagtggtgtgacaaaatttagtggcatggttctactgtgtaaagtctgtggggatgtggcgtcaggattccactatggagttcatgcttgcgaaggctgtaagggtttctttcggagaagtattcaacaaaacatccagtacaagaagtgcctgaagaatgaaaactgttctataatgagaatgaataggaacagatgtcagcaatgtcgcttcaaaaagtgtctgtctgttggaatgtcaagagatgctgttcggtttggtcgtattcctaagcgtgaaaaacagaggatgctaattgaaatgcaaagtgcaatgaagaccatgatgaacagccagttcagtggtcacttgcaaaatgacacattagtagaacatcatgaacagacagccttgccagcccaggaacagctgcgacccaagccccaactggagcaagaaaacatcaaaagctcttctcctccatcttctgattttgcaaaggaagaagtgattggcatggtgaccagagctcacaaggatacctttatgtataatcaagagcagcaagaaaactcagctgagagcatgcagccccagagaggagaacggattcccaagaacatggagcaatataatttaaatcatgatcattgcggcaatgggcttagcagccattttccctgtagtgagagccagcagcatctcaatggacagttcaaagggaggaatataatgcattacccanatggccatgccatttgtattgcaaatggacattgtatgaacttctccaatgcttatactcaaagagtatgtgatagagttccgatagatggattttctcagaatgagaacaagaatagttacctgtgcaacactggaggaagaatgcatctggtttgtccaatgagtaagtctccatatgtggatcctcataaatcaggacatgaaatctgggaagaattttcgatgagcttcactccagcagtgaaagaagtggtggaatttgcaaagcgtattcctgggttcagagatctctctcagcatgaccaggtcaaccttttaaaggctgggacttttgaggttttaatggtacggttcgcatcattatttgatgcaaaggaacgtactgtcacctttttaagtggaaagaaatatagtgtggatgatttacactcaatgggagcaggggatctgctaaactctatgtttgaatttagtgagaagctaaatgccctccaacttagtgatgaagagatgagtttgtttacagctgttgtcctggtatctgcagatcgatctggaatagaaaacgtcaactctgtggaggctttgcaggaaactctcattcgtgcactaaggaccttaataatgaaaaaccatccaaatgaggcctctatttttacaaaactgcttctaaagttgccagatcttcgatctttaaacaacatgcactctgaggagctcttggcctttaaagttcacccttaa

SEQ ID NO: 6 - protein, 579 aa, Homo sapiens
MEVNAGGVIAYISSSSSASSPASCHSEGSENSFQSSSSSVPSSPNSSNSDTNGNPKNGDLANIEGILKNDRIDCSMKTSKSSAPGMTKNHSGVTKFSGMVLLCKVCGDVASGFHYGVHACEGCKGFFRRSIQQNIQYKKCLKNENCSIMRMNRNRCQQCRFKKCLSVGMSRDAVRFGRIPKREKQRMLIEMQSAMKTMMNSQFSGHLQNDTLVEHHEQTALPAQEQLRPKPQLEQENIKSSSPPSSDFAKEEVIGMVTRAHKDTFMYNQEQQENSAESMQPQRGERIPKNMEQYNLNHDHCGNGLSSHFPCSESQQHLNGQFKGRNIMHYPXGHAICIANGHCMNFSNAYTQRVCDRVPIDGFSQNENKNSYLCNTGGRMHLVCPMSKSPYVDPHKSGHEIWEEFSMSFTPAVKEVVEFAKRIPGFRDLSQHDQVNLLKAGTFEVLMVRFASLFDAKERTVTFLSGKKYSVDDLHSMGAGDLLNSMFEFSEKLNALQLSDEEMSLFTAVVLVSADRSGIENVNSVEALQETLIRALRTLIMKNHPNEASIFTKLLLKLPDLRSLNNMHSEELLAFKVHP

SEQ ID NO: 7 - RNA, 2058 nt, Homo sapiens
  source 1..2058: mol_type = mRNA
atggcggcagcgtcccggagcgcctctggctgggcgctactgctgctggtggcactttggcagcagcgcgcggccggctccggcgtcttccagctgcagctgcaggagttcatcaacgagcgcggcgtactggccagtgggcggccttgcgagcccggctgccggactttcttccgcgtctgccttaagcacttccaggcggtcgtctcgcccggaccctgcaccttcgggaccgtctccacgccggtattgggcaccaactccttcgctgtccgggacgacagtagcggcggggggcgcaaccctctccaactgcccttcaatttcacctggccgggtaccttctcgctcatcatcgaagcttggcacgcgccaggagacgacctgcggccagaggccttgccaccagatgcactcatcagcaagatcgccatccagggctccctagctgtgggtcagaactggttattggatgagcaaaccagcaccctcacaaggctgcgctactcttaccgggtcatctgcagtgacaactactatggagacaactgctcccgcctgtgcaagaagcgcaatgaccacttcggccactatgtgtgccagccagatggcaacttgtcctgcctgcccggttggactggggaatattgccaacagcctatctgtctttcgggctgtcatgaacagaatggctactgcagcaagccagcagagtgcctctgccgcccaggctggcagggccggctgtgtaacgaatgcatcccccacaatggctgtcgccacggcacctgcagcactccctggcaatgtacttgtgatgagggctggggaggcctgttttgtgaccaagatctcaactactgcacccaccactccccatgcaagaatggggcaacgtgctccaacagtgggcagcgaagctacacctgcacctgtcgcccaggctacactggtgtggactgtgagctggagctcagcgagtgtgacagcaacccctgtcgcaatggaggcagctgtaaggaccaggaggatggctaccactgcctgtgtcctccgggctactatggcctgcattgtgaacacagcaccttgagctgcgccgactccccctgcttcaatgggggctcctgccgggagcgcaaccagggggccaactatgcttgtgaatgtccccccaacttcaccggctccaactgcgagaagaaagtggacaggtgcaccagcaacccctgtgccaacgggggacagtgcctgaaccgaggtccaagccgcatgtgccgctgccgtcctggattcacgggcacctactgtgaactccacgtcagcgactgtgcccgtaacccttgcgcccacggtggcacttgccatgacctggagaatgggctcatgtgcacctgccctgccggcttctctggccgacgctgtgaggtgcggacatccatcgatgcctgtgcctcgagtccctgcttcaacagggccacctgctacaccgacctctccacagacacctttgtgtgcaactgcccttatggctttgtgggcagccgctgcgagttccccgtgggcttgccgcccagcttcccctgggtggccgtctcgctgggtgtggggctggcagtgctgctggtactgctgggcatggtggcagtggctgtgcggcagctgcggcttcgacggccggacgacggcagcagggaagccatgaacaacttgtcggacttccagaaggacaacctgattcctgccgcccagcttaaaaacacaaaccagaagaaggagctggaagtggactgtggcctggacaagtccaactgtggcaaacagcaaaaccacacattggactataatctggccccagggcccctggggcgggggaccatgccaggaaagtttccccacagtgacaagagcttaggagagaaggcgccactgcggttacacagtgaaaagccagagtgtcggatatcagcgatatgctcccccagggactccatgtaccagtctgtgtgtttgatatcagaggagaggaatgaatgtgtcattgccacggaggtataa

SEQ ID NO: 8 - protein, 685 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 9 - DNA, 426 bp, Homo sapiens
  source 1..426: mol_type = other DNA
atgtcaaacatgagatgtgtggactgtggcacttgcctgggtcacacacggaggcatcctacccttttctggggaaagacactgcctgggctgaccccggtggcggccccagcacctcagcctgcacagtgtcccccaggttccgaagaagatgctccagcaacacagcctgggccccagctcgcgggacccgaccccccgtgggctcccgtgttttgtaggagacttgccagagccgggcacattgagctgtgcaacgccgtgggctgcgtcctttggtcctgtccccgcagccctggcagggggcatgcggtcgggcaggggctggagggaggcgggggctgcccttgggccacccctcctagtttgggaggagcagatttttgcaataccaagtatagcctatggcagaaaaaatgtctttaa

SEQ ID NO: 10 - protein, 141 aa, Homo sapiens
MSNMRCVDCGTCLGHTRRHPTLFWGKTLPGLTPVAAPAPQPAQCPPGSEEDAPATQPGPQLAGPDPPWAPVFCRRLARAGHIELCNAVGCVLWSCPRSPGRGHAVGQGLEGGGGCPWATPPSLGGADFCNTKYSLWQKKCL